(19) United States
(12) Patent Application Publication
(10) Pub. No.: US 2005/0069863 A1
(43) Pub. Date: Mar. 31, 2005

(54) Systems and methods for analyzing gene expression data for clinical diagnostics

(76) Inventors: Jorge Moraleda, Menlo Park, CA (US); Glenda G. Anderson, San Jose, CA (US)

Correspondence Address: JONES DAY, 222 EAST 41ST ST, NEW YORK, NY, 10017, US

(21) Appl. No.: 10/861,216

(22) Filed: Jun. 4, 2004

Related U.S. Application Data
(60) Provisional application No. 60/507,381, filed on Sep. 29, 2003.

(51) Int. Cl. C12Q 1/00; C12Q 1/68; G06F 19/00; G01N 33/48; G01N 33/50

(52) U.S. Cl. 435/4; 702/19

(57) ABSTRACT

Methods, computer program products and computer systems for constructing a classifier for classifying a specimen into a class are provided. The classifiers are models. Each model includes a plurality of tests. Each test specifies a mathematical relationship (e.g., a ratio) between the characteristics of specific cellular constituents. Each test is polled using characteristic values of these specified cellular constituents from the biological specimen to be classified. In some embodiments, each test has a positive threshold and a negative threshold. When the value of the test exceeds the positive threshold, the test polls positive. When the value of the test is below the negative threshold, the test polls negative. When the value of the test is between the negative threshold and the positive threshold, the test polls indeterminate. The value of each test is combined to provide a composite score. In some embodiments, positive composite scores indicate that the specimen belongs in the class associated with the model.

### CROSS-REFERENCE TO RELATED APPLICATIONS

This application claims benefit, under 35 U.S.C. § 119(e), of U.S. Provisional Patent Application No. 60/507,381, filed on Sep. 29, 2003, which is hereby incorporated by reference in its entirety.

### 1. FIELD OF THE INVENTION

The field of this invention relates to computer systems and methods for classifying a biological specimen.

### 2. BACKGROUND OF THE INVENTION

Current bioinformatics tools recently applied to microarray data have shown utility in predicting both cancer diagnosis and outcome. See, for example, Golub et al., 1999, Science 286, p. 531; and Pomeroy et al., 2002, Nature 415, p. 436. However, their widespread relevance and applicability are unresolved. For example, the discrimination function can vary (for the same genes) based on the location and protocol used for sample preparation. See, for example, Golub et al., 1999, Science 286, p. 531. Further, profiling with a microarray requires relatively large quantities of RNA, making the process inappropriate for certain applications. Also, it has yet to be determined whether these approaches can use relatively low-cost and widely applicable data acquisition platforms such as real-time quantitative polymerase chain reaction (RT-PCR) and still retain significant predictive capabilities. Another limitation in translating microarray profiling to patient care is that this approach cannot currently be used to diagnose individual samples independently without comparison with a predictor model generated from samples of the data that were acquired on the same platform.

To address these limitations in the art, Gordon et al., 2002, Cancer Research 62, p. 4963 (Gordon 2002) explored an alternative approach using gene expression measurements to predict clinical parameters in cancer. In particular, Gordon 2002 explored the feasibility of a test that uses ratios of gene expression levels to distinguish between malignant pleural mesothelioma (MPM) and adenocarcinoma (ADCA) of the lung. cRNA was prepared from total RNA of discarded MPM and ADCA surgical specimens and hybridized to microarrays. The microarray data was processed and negative values on the microarray were converted to their absolute value. To generate graphical representations of relative gene expression levels, all of the expression levels were first normalized within samples by setting the average (median) to zero and the standard deviation to one.

All the genes represented on such microarrays were searched for those with highly significant differences (>8-fold) in average expression levels between the two tumor types in the training set of 16 ADCA and 16 MPM samples. From this set, eight genes with the most statistically significant differences and a mean expression level >600 in at least one of the two training samples sets were selected.

Of the eight genes selected in Gordon 2002, five expressed at relatively higher levels in MPM and three expressed at relatively higher levels in ADCA tumors. The eight genes define fifteen ratios in which the five genes expressed at relatively higher levels in MPM are divided by each of the three genes expressed at relatively higher levels in ADCA. The fifteen ratios were tested against samples not included in the training set. Samples with ratio values >1 were called MPM and those with ratio values <1 were called ADCA. The fifteen ratios correctly distinguished between the MPM and ADCA tumor types in the samples not included in the training set with an accuracy ranging from 91% for the least accurate ratio to 98% for the most accurate ratio where accuracy is defined as the fraction of tumors in the population that were diagnosed correctly.

To improve the accuracy of the method, Gordon 2002 further proposed the use of a pair of ratios from the set of fifteen ratios. When the pair of ratios were in disagreement, a third ratio was used to resolve the discrepancy. Using this best of three polling approach, 99 percent accuracy was achieved in distinguishing between the MPM and ADCA tumor types in the samples not included in the training set. In Gordon 2003, Journal of the National Cancer Institute 95, p. 598 (Gordon 2003), the method used to combine ratios to provide a more accurate classifier was modified. In Gordon 2003, data from three individual gene pair ratios that predicted the group membership of training set samples with the highest accuracy were combined by calculating a geometric mean, (R₁R₂R₃)^{1/3}, of the ratios, where Rn represents a single value and direction (>1 and <1) of the geometric mean is used to classify a sample.

Although Gordon 2002 and Gordon 2003 represent significant accomplishments in the art in their own right, there are drawbacks to the techniques described in these references. In Gordon 2002 and Gordon 2003, genes are selected for use in ratios based on differences in mean expression values between biological classes. Thus, the selection process is dependent upon the presence of genes that have significant differences of expression between biological classes. However, as illustrated in Gordon 2002, genes that have significant differential expression between two biological classes are not always available. In Gordon 2002, a set of 60 medullogblastoma tumors with linked clinical data were obtained from the published microarray data of Pomeroy et al., 2002, Nature 415, p. 436. Of these 60 samples, 39 and 21 originated from patients classified as “treatment responders” and “treatment failures”, respectively. A training set of 20 randomly chosen samples (10 responders and 10 failures) were used to identify predictor genes. However, because of the paucity of genes that had significantly different expression in the “treatment responders” and “treatment failures” classes reduced filtering criteria (>2-fold change in average expression levels, and at least one mean >200 for one of the two classes) were used to select genes for use in ratios. The most significant three genes expressed at relatively higher levels in each group were used to form a set of nine ratios. The accuracy of these nine ratios was only in the range of 43-70 percent, where accuracy is defined as the percentage of correctly predicted samples not in the training set. When the geometric mean of all nine ratios was combined in the manner described in more detail in Gordon 2003, the accuracy was 68 percent. This result is lower than the 78 percent accuracy achieved by Pomeroy et al., 2002, Nature 415, p. 436, using non-ratio based methods.

Another drawback with Gordon 2002 and 2003 is the binary method by which a ratio is evaluated, when the ratio is <1, it is designated the first class and when the ratio is >1 it is designated the second class. Thus, ratio calculations that are marginal can, in fact, control the final determination. Still another drawback with Gordon 2002 and 2003 is that such methods do not protect against, and in fact encourage the use of, extreme gene expression values. Such values are often the least stable from experiment to experiment.

Thus, given the above background, what is needed in the art are improved methods for classifying specimens into biological classes using ratio-based classifiers.

Discussion or citation of a reference herein will not be construed as an admission that such reference is prior art to the present invention.

### 3. SUMMARY OF THE INVENTION

Novel advancements in the art are provided. In the present invention, several different methods for building classifiers are provided. In some embodiments, the classifiers are organized into suites of models. In some embodiments, the classifiers are individual models. Regardless of whether or not the models are organized into suites, each model is designed to detect the presence or absence of a specific biological feature. In the present invention, a specific biological feature includes, but is not limited to, the absence or presence of a disease, an indication of a specific tissue type (e.g., lung), or an indication of disease origin. Each model comprises a set of tests. For example, a model can comprise one, two, three, four, five, or more than five tests. Each test polls the cellular constituent characteristic of one or more specific cellular constituents in the specimen or biological sample to be classified. In some embodiments, each test consists of the ratio of the characteristic of a specific first cellular constituent divided by the ratio of a specific second cellular constituent. In other embodiments, each test comprises the characteristic of a specific cellular constituent, the product of two cellular constituents, or some other mathematical operation on one or more cellular constituents.

Common to all tests of the present invention is the use of positive and negative thresholds. That is, each test in each model of the present invention is assigned a positive threshold and a negative threshold. When a polled test returns a value that exceeds its positive threshold, the test provides a positive vote. When a polled test returns a value that is below the positive threshold but above the negative threshold, the test is indeterminate and provide a vote of “0”. When a polled test returns a value that is below its negative threshold, the test returns a negative vote. A test is polled by inserting the cellular constituent characteristic values specified into the test from the target specimen or biological sample. For example, is a test is the ratio of a characteristic (e.g., abundance) of cellular constituent A divided by a characteristic (e.g., abundance) of cellular constituent B, the test is polled by obtaining the characteristic of cellular constituent A and B from the specimen or biological organism to be polled and taking their ratio. In some embodiments, positive votes and negative votes are “+1” and “−1”, respectively. In some embodiments, positive votes and weighted by some measure of confidence in the test wherein the positive vote can range from near zero to some value larger than “1”. In some embodiments, negative votes are also weighted by some measure of confidence in the test so that the negative vote can range from near zero to some value less than “−1”.

Models are scored by summing each polled test in the model. A positive summation of the model indicates that the organism or biological specimen associated with the model has the phenotypic feature associated with the model. A null or negative summation of the model indicates that the organism or biological specimen associated with the model does not have the phenotypic feature associated with the model.

The indeterminate region found in each of the tests of the present invention are highly advantageous. They improve the accuracy of the model by removing a test from consideration when the results of the poll of the test fall into a range of values that has been determined to lack predictive power. The present invention provides a number of different methods for identifying the indeterminate region of each test. These include a “True Minimum/False Maximum” approach summarized in Section 3.1 and other approaches summarized in Sections 3.2 through 3.4.

### 3.1. True Minimum/False Maximum Approach

At the outset, a cellular constituent dataset from each biological specimen considered is optionally standardized by dividing each cellular constituent characteristic value in the cellular constituent dataset by the median cellular constituent characteristic value of the dataset (the median cellular constituent characteristic value of the cellular constituents from the biological specimen corresponding to the biological specimen).

Next, the cellular constituents that have been identified as uniquely associated with a particular biological class among the biological classes to be differentiated are considered as candidate cellular constituents. For example, in some instances, clustering analysis can identify a set of cellular constituents {A} that are up-regulated in a first biological class and a set of cellular constituents {B} that are up-regulated in a second biological class relative to another biological sample class.

Cellular constituent pairs, selected from those cellular constituents that are uniquely associated with a particular biological class, are evaluated as ratios by the methods of the present invention in order to cellular constituent pairs that are suitable for use as classifiers. For example cellular constituents A and B may be tested in ratio form, A/B, to determine whether the are suitable for use in a classifier. In one case, using the example presented above, each possible cellular constituent pair is considered in ratio form, where the numerator (first member of the pair) is selected from the set {A} and a denominator (second member of the pair) is selected from the set {B}. For each cellular constituent pair considered as a ratio, the cellular constituent characteristic values from a plurality of specimens with known classification are used to generate a corresponding set of ratios having the same numerator and denominator of the given ratio. For example, if the given ratio is A₁/B₁ (corresponding to the ratio pair A₁, B₁) then the cellular constituent characteristic values for A₁ and B₁ from a first biological specimen form a first ratio in the corresponding set of ratios, the cellular constituent characteristic values for A₁ and B₁ from a second biological specimen form a second ratio in the corresponding set of ratios, and so forth. The set of cellular constituents corresponding to the given ratio are divided into two subsets, the true values and the false values. The true values represent those ratios in the corresponding set that were calculated using characteristic values (e.g., abundances) from a specimen in which the numerator (A₁) is up-regulated. The false values represent those ratios that were calculated using characteristic values from a specimen in which the numerator (A₁) is not up-regulated. A distribution of the true values is made. Likewise a distribution of the false values is made. The distribution of the true values is used to calculate a true minimum (e.g., 20^{th} percentile of the true values) and the distribution of the false values is used to calculate a false maximum (e.g., 90^{th} percentile of false values). The true minimum and false maximum are associated with the cellular constituent pair that determines the given ratio.

At this stage, a large number of cellular constituent pairs have been considered as ratios. Each ratio (and therefore cellular constituent pairs corresponding to such ratios) is uniquely associated with a true minimum and a false maximum using the approach described above. Because each cellular constituent data set used in the computation of the true minimum and false maximum has been standardized (by dividing the dataset by the median cellular constituent characteristic value of the originating specimen), the true minimum and false maximum can be applied uniformly as filters to remove ratios (and effectively the cellular constituent pairs that determine such ratios) from consideration as classifiers. For example, in some embodiments, a ratio is removed from consideration if the true minimum for the ratio is not greater than the false maximum.

Standardization of the cellular constituent characteristic data (e.g., abundance data) allows for the application of other novel filters. In some embodiments, ratios are removed from consideration when the value of the numerator is not greater than a threshold value, such as two. This drives for selection of ratios (and their corresponding cellular constituent pairs) in which the numerator represents a cellular constituent that has a characteristic that is at least twice the median value of the characteristics (e.g., abundances) of cellular constituents in the originating specimen.

The true minimum and false maximum for each ratio that is selected for a classifier are used to define a novel indeterminate region. The indeterminate region is that region that is greater than the false maximum and less than the true minimum. When a classifier ratio is calculated using cellular constituent characteristic data from a test specimen and this calculation results in a value in the indeterminate region the ratio is not used to perform a classification. In this way ratios that produce indeterminate values can be underweighted or ignored in polling the sets of ratios of a classifier in order to establish improved accuracy.

The present invention provides methods, computer program products and computer systems for constructing classifiers that classify a specimen into one of a plurality of classes. The invention further provides methods, computer program products and computer systems for using such classifiers to classify specimens into biological classes.

To construct a classifier for a given class, a plurality of test ratios are calculated for a given class in a plurality of classes. The numerator and denominator of each ratio in the plurality of test ratios represent a cellular constituent pair and are respectively determined by a characteristic of a first and second cellular constituent measured from the same biological specimen. Further, at least one of the first and second cellular constituent are either up-regulated or down-regulated in the given biological sample class relative to another biological sample class. More than one biological sample class is represented in the plurality of test ratios.

Next, set of cellular constituent pairs for the given biological sample class is selected from the cellular constituent pairs uses to construct the plurality of test ratios. When properly selected, the set of cellular constituent pairs serves as a classifier. The present invention provides a number of criteria used to facilitate selection of cellular constituent pairs for the set of cellular constituent pairs. To consider a given cellular constituent pair for inclusion in the set, a distribution of a first plurality of test ratios and a distribution of a second plurality of test ratios is calculated. The numerator and denominator of each test ratio in the first and second plurality of test ratios is respectively determined by characteristics (e.g., abundances) of the first and second cellular constituent in a candidate cellular constituent pair. Characteristics used for the first plurality of test ratios are from members of the respective biological sample class. Characteristics for the second plurality of test ratios are not from members of the respective biological sample class. When a lower threshold percentile from the distribution of the first plurality of test ratios is greater than an upper threshold percentile from the distribution of the second plurality of test ratios, the given cellular constituent pair that determines the ratio is a candidate for inclusion in the set of cellular constituent pairs.

### 3.2. Models Comprising Tests in Which Each Test has a Positive Threshold and a Negative Threshold

One aspect in accordance with the present invention provides a computer program product for use in conjunction with a computer system. The computer program product comprises a computer readable storage medium and a computer program mechanism embedded therein. The computer program mechanism comprises a model characterized by a model score, the model comprising a plurality of tests. Each respective test in the plurality of tests is characterized by a test value that is determined by a function of the characteristics (e.g., abundances) of one or more cellular constituents in a plurality of cellular constituents in a test organism of a species or a test biological specimen from an organism of the species. Each respective test in the plurality of tests is independently assigned a positive threshold and a negative threshold so that
(i) the respective test positively contributes to the model score when the test value for the respective test exceeds the positive threshold;
(ii) the respective test does not contribute to the model score when the test value for the respective test is less than the positive threshold and greater than the negative threshold; and
(iii) the respective test negatively contributes to the model score when the test value for the respective test is less than the negative threshold.

In some embodiments, the function of a test in the plurality of tests comprises a ratio between a numerator and a denominator, wherein the numerator comprises a characteristic of a predetermined first cellular constituent in the test organism or test biological specimen and the denominator comprises a characteristic (e.g., abundance) of a predetermined second cellular constituent in the test organism or test biological specimen. In such embodiments,
(i) the test positively contributes to the model score when the ratio exceeds the positive threshold;
(ii) the test does not contribute to the model score when the ratio is less than the positive threshold and greater than the negative threshold; and
(iii) the test negatively contributes to the model score when the ratio is less than the negative threshold.

In some embodiments, the model represents the absence or presence of a biological feature in the test organism or the test biological specimen, and
the test organism or the test biological specimen is deemed to have the biological feature when the model score is positive; and
the test organism or the test biological specimen is deemed to not have the biological feature when the model score is negative.

In some embodiments, the function of a test in the plurality of tests comprises a ratio between a numerator and a denominator. In such embodiments, the numerator comprises a characteristic (abundance) of a predetermined first cellular constituent in the test organism or test biological specimen and the denominator comprises a characteristic (e.g., abundance) of a predetermined second cellular constituent in the test organism or test biological specimen. Further, the first cellular constituent is more abundant in members of the species or biological specimens that have the biological feature than in members of the species that do not have the biological feature. The second cellular constituent is less abundant in members of the species or biological specimens that have the biological feature than in members of the species or biological specimens that do not have the biological feature.

In some embodiments, the plurality of tests comprises a first test and a second test and the identities of the one or more cellular constituents whose characteristics (e.g., abundances) in the test organism or test biological specimen used to determine the value of the first test are different than the identities of the one or more cellular constituents whose characteristics in the test organism or test biological specimen used to determine the value of the second test.

In some embodiments, the plurality of tests comprises a first test and a second test and an identity of a cellular constituent in the one or more cellular constituents whose characteristics are used to determine the value of the first test is the same as the identity of a cellular constituent in the one or more cellular constituents whose characteristics are used to determine the value of the second test.

In some embodiments, a test in the plurality of tests contributes
a single positive unit to the model score when the test value for the test exceeds the positive threshold assigned to the test;
zero units to the model score when the test value for the test is less than the positive threshold assigned to the test and greater than the negative threshold assigned to the test; and
a single negative unit to the model score when the test value for the test is less than the negative threshold assigned to the test.

In some embodiments, a test in the plurality of tests contributes (i) a weighted positive unit to the model score when the test value for the test exceeds the positive threshold assigned to the test, (ii) zero units to the model score when the test value for the test is less than the positive threshold assigned to the test and greater than the negative threshold assigned to the test, (iii) and a weighted negative unit to the model score when the test value for the test is less than the negative threshold assigned to the test. In some embodiments, the magnitude of the weighted positive unit is determined by an amount the test value exceeds the positive threshold assigned to the test. In some embodiments, the magnitude of the weighted positive unit and the weighted negative unit is determined by a degree of confidence in the test. In some embodiments, the magnitude of the weighted positive unit and the weighted negative unit is determined by an area under a receiver operating characteristic (ROC) curve used to assign the positive threshold and the negative threshold to the test. In some embodiments, the magnitude of the weighted negative unit is determined by an amount the test value is less than the negative threshold assigned to the test.

In some embodiments, the computer program product further comprises a cellular constituent data set and instructions for using the cellular constituent data set to assign a positive threshold and a negative threshold to a test in the plurality of tests. In some embodiments, the cellular constituent data set comprises
a plurality of cellular constituent characteristic measurements from (i) each organism in a plurality of organisms of the species, or (ii) each biological specimen in a plurality of biological specimens from organisms of the species; and
an indication whether, for each respective organism in the plurality of organisms or for each respective organism corresponding to a biological specimen in the plurality of biological specimens, a biological feature is present or absent in the respective organism.

In some embodiments, the instructions for using the cellular constituent data set to assign a positive threshold and a negative threshold to a test in the plurality of tests comprises selecting:
a first subset of the plurality of cellular constituents, wherein each cellular constituent in the first subset of cellular constituents is up-regulated in organisms in which the biological feature is present; and
a second subset of the plurality of cellular constituents, wherein each cellular constituent in the second subset of cellular constituents is down-regulated in organisms in which the biological feature is present.

In some embodiments, the instructions for using the cellular constituent data set to assign a positive threshold and a negative threshold to a test in the plurality of tests comprises
constructing a test in the plurality of tests, wherein the function of the test is a ratio between (i) a characteristic (e.g., abundance) of a cellular constituent in the first subset and (ii) a characteristic (e.g., abundance) of a cellular constituent in the second subset.

### 3.3. The use of Mutual Information to Select Cellular Constituents for use in Diagnostic Models

Another aspect of the present invention provides a computer program product for use in conjunction with a computer system. The computer program product comprises a computer readable storage medium and a computer program mechanism embedded therein. The computer program product comprises (A) instructions for accessing one or more data structures collectively comprising a cellular constituent characteristic (e.g., abundance) of each cellular constituent in a plurality of cellular constituents measured in a biological specimen from each member of a population of a species. This population includes members that have a biological feature and members that do not have the biological feature. The computer program product further comprises (B) instructions for determining a distribution p(xᵢ) of the biological feature across all or a portion of the population, wherein for each member i represented by the distribution p(xᵢ),
xᵢ takes a first value when the specimen indexed by i has the biological feature; and
xᵢ takes a second value when the specimen indexed by i does not have the biological feature.
   The computer program product further comprises (C) instructions for determining a distribution q(yᵢ) of characteristic values for a cellular constituent Y in the plurality of cellular constituents across all or a portion of the population. The computer program product further comprises (D) instructions for computing a mutual information score I(X,Y) between X and Y and instructions for repeating the instructions (C) and (D) for one or more cellular constituents in the plurality of cellular constituents thereby identifying a cellular constituent Y such that the mutual information between X and Y is larger than that between X and one or more other cellular constituents in the plurality of cellular constituents.

In some embodiments, the computer program product further comprises instructions for dividing the data structure into a training data set partition and a test data set partition wherein
the training data set partition comprises cellular constituent characteristics of the plurality of cellular constituents measured in biological specimens from a randomly selected first subset of the population; and
the test data set partition comprises cellular constituent characteristics (e.g., abundances) of the plurality of cellular constituents measured in biological specimens from a randomly selected second subset of the population, provided that biological specimens represented by the second subset are not represented by the first subset; and wherein
the portion of the population considered by the instructions for determining (B) and the instructions for determining (C) is the training data set partition.

In some embodiments

$$I ( X , Y ) = H ( X ) - H ( X \vert Y ) = {\sum }_{x , y}^{} r ( x , y ) {log}_{2} \frac{r ( x , y )}{xy}$$

wherein,
H(X) is the entropy of the random variable X that represents the presence or absence of a biological feature;
H(X|Y) is the entropy of the random variable X given the random variable Y, where Y's values correspond to the characteristic (e.g., abundance) of a cellular constituent i across all or a portion of the population; and
r(x,y) is the joint distribution of X and Y.

In some embodiments, the computer program product further comprises instructions for ranking a plurality of cellular constituents tested by instances of the instructions for determining (C) and the instructions for computing (D) by the respective mutual information scores of the one or more cellular constituents computed by the instructions for computing (D) in order to form a ranked list of cellular constituents. In such embodiments, the computer program product further includes instructions for selecting a plurality of cellular constituents from a top-ranked portion of the ranked list of cellular constituents for inclusion in a model that is diagnostic of the biological feature.

In some embodiments, the top-ranked portion of the ranked list of cellular constituent is the first five cellular constituents in the ranked list, the first ten cellular constituents in the ranked list, the first twenty cellular constituents in the ranked list, the first one hundred cellular constituents in the ranked list, the upper one percent of the cellular constituents in the ranked list, the upper three percent of the cellular constituents in the ranked list, or the upper ten percent of the cellular constituents in the ranked list.

In some embodiments, the instructions for selecting cellular constituents comprises instructions for dividing the top-ranked portion of the ranked list into a first category and a second category wherein
cellular constituents in the first category are those cellular constituents whose characteristic values in all or the portion of the population positively correlate with X; and
cellular constituents in the second category are those cellular constituents whose characteristic values in all or the portion of the population negatively correlate with the distribution X.

In some embodiments, the instructions for selecting cellular constituents further comprises instructions for constructing the model, wherein the model comprises a plurality of tests and wherein each test includes a first cellular constituent in the first category and a second cellular constituent in the second category. In some embodiments, the first cellular constituent in each test in the model is different. In some embodiments, the second cellular constituent in each test in the model is different.

In some embodiments, the model is characterized by a model score and each respective test in the plurality of tests is characterized by a test value that is determined by a function of the characteristic (e.g., abundance) of the first cellular constituent and the characteristic of the second cellular constituent in a test biological specimen from an organism.

In some embodiments, the function of a test in the plurality of tests is a ratio in which the characteristic of the first cellular constituent is the numerator of the ratio and the characteristic of the second cellular constituent is the denominator of the ratio. In such embodiments,
the test positively contributes to the model score when the ratio exceeds the positive threshold;
the test does not contribute to the model score when the ratio is less than the positive threshold and greater than the negative threshold; and
the test negatively contributes to the model score when the ratio is less than the negative threshold.

In some embodiments, each respective test in the plurality of tests is independently assigned a positive threshold and a negative threshold so that
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold.

In some embodiments, the model represents the absence or presence of a biological feature in the test biological specimen and (i) the test biological specimen is deemed to have the biological feature when the model score is positive, and (ii) the test biological specimen is deemed to not have the biological feature when the model score is negative.

In some embodiments, the computer program product further comprises instructions for validating the model by quantifying the specificity or the sensitivity of the model against the cellular constituent characteristic data of a portion of the population of the species not used to assign a positive threshold or a negative threshold to a test in the plurality of tests in the model.

Another aspect of the invention provides a method comprising the steps of:
(A) accessing cellular constituent characteristic data for each cellular constituent in a plurality of cellular constituents measured in a biological specimen from each member of a population of a species, wherein the population includes members that have a biological feature and members that do not have the biological feature;
(B) determining a distribution p(xᵢ) of the biological feature across all or a portion of the population, wherein for each member i represented by the distribution p(xᵢ),
   xᵢ takes a first value when the specimen represented by i has the biological feature; and
   xᵢ takes a second value when the specimen represented by i does not have the biological feature;
(C) determining a distribution q(yᵢ) of characteristic values for a cellular constituent Y in the plurality of cellular constituents across all or a portion of the population;
(D) determining a mutual information score I(X,Y) between X and Y; and
(E) repeating the determining (C) and the determining (D) for one or more cellular constituents in the plurality of cellular constituents thereby identifying a cellular constituent Y wherein the mutual information between X and Y is larger than that between X and one or more other cellular constituents in the plurality of cellular constituents.

### 3.4. The use of Receiver Operating Characteristic Curves to Determine Diagnostic Model Threshold Values

Another aspect of the invention provides a computer program product for use in conjunction with a computer system. The computer program product comprises a computer readable storage medium and a computer program mechanism embedded therein. The computer program mechanism comprises a model characterized by a model score (or instructions for accessing the model). The model comprises a plurality of tests. Each respective test in the plurality of tests is characterized by a test value that is determined by a function of the characteristic of one or more cellular constituents in a plurality of cellular constituents in a test organism of a species or a test biological specimen from an organism of the species. The computer program mechanism further comprises instructions for identifying one or more candidate thresholds for each respective test in the plurality of tests. The computer program product further comprises instructions for scoring each candidate threshold combination in a plurality of candidate threshold combinations. Each candidate threshold combination in the plurality of candidate threshold combinations comprises one or more candidate thresholds for each test in the plurality of tests that was identified by the instructions for identifying.

In some embodiments, the instructions for identifying one or more candidate thresholds for each respective test in the plurality of tests comprises instructions for identifying a positive threshold and a negative threshold for each respective test in the plurality of tests so that each respective test:
positively contributes to the model score when the test value for the respective test exceeds the positive threshold;
does not contribute to the model score when the test value for the respective test is less than the positive threshold and greater than the negative threshold; and
negatively contributes to the model score when the test value for the respective test is less than the negative threshold.

In some embodiments, the function of a test in the plurality of tests comprises a characteristic of a predetermined cellular constituent; wherein
the test positively contributes to the model score when the characteristic of the cellular constituent in the test organism or the test biological specimen exceeds the positive threshold;
the test does not contribute to the model score when the characteristic of the cellular constituent in the test organism or the test biological specimen is less than the positive threshold and greater than the negative threshold; and
the test negatively contributes to the model score when the characteristic of the cellular constituent in the test organism or the test biological specimen is less than the negative threshold.

In some embodiments, the function of a test in the plurality of tests comprises a ratio between a numerator and a denominator, wherein the numerator comprises a characteristic of a predetermined first cellular constituent in the test organism or test biological specimen and the denominator comprises a characteristic of a predetermined second cellular constituent in the test organism or test biological specimen. In such embodiments,
the test positively contributes to the model score when the ratio exceeds the positive threshold;
the test does not contribute to the model score when the ratio is less than the positive threshold and greater than the negative threshold; and
the test negatively contributes to the model score when the ratio is less than the negative threshold.

In some embodiments, the model represents the absence or presence of a biological feature in the test organism or the test biological specimen such that:
the test organism or the test biological specimen is deemed to have the biological feature when the model score is positive; and
the test organism or the test biological specimen is deemed to not have the biological feature when the model score is negative.

In some embodiments, a test in the plurality of tests contributes:
a weighted positive unit to the model score when the test value for the test exceeds the positive threshold assigned to the test;
zero units to the model score when the test value for the test is less than the positive threshold assigned to the test and greater than the negative threshold assigned to the test; and
a weighted negative unit to the model score when the test value for the test is less than the negative threshold assigned to the test.
   In some embodiments, the magnitude of the weighted positive unit is determined by an amount the test value exceeds the positive threshold assigned to the test. In some embodiments, the magnitude of the weighted positive unit and the weighted negative unit is determined by a degree of confidence in the test. In some embodiments, the magnitude of the weighted positive unit and the weighted negative unit is determined by an area under a receiver operating characteristic (ROC) curve used to assign the positive threshold and the negative threshold to the test. In still other embodiments, the magnitude of the weighted negative unit is determined by an amount the test value is less than the negative threshold assigned to the test.

In some embodiments the computer program product further comprises instructions for accessing a cellular constituent data set, the cellular constituent data set comprising:
a plurality of cellular constituent characteristic measurements from (i) each organism in a plurality of organisms of the species, or (ii) each biological specimen in a plurality of biological specimens from organisms of the species; and
an indication whether, for each respective organism in the plurality of organisms or for each respective organism corresponding to a biological specimen in the plurality of biological specimens, a biological feature is present or absent in the respective organism; and
the instructions for identifying one or more candidate thresholds for each respective test in the plurality of tests comprises:
   (i) instructions for computing the function of a respective test in the plurality of tests using the characteristics (e.g., abundances) of the one or more cellular constituents that determine the test value of the respective test, wherein the characteristics (e.g., abundances) of the one or more cellular constituents are from an organism in the plurality of organisms or a biological specimen in the plurality of biological specimens in the cellular constituent data set;
   (ii) instructions for repeating the instructions for computing (i) using the characteristics of the one or more cellular constituents that determine the test value from a different organism in the plurality of organisms or the biological specimen in the plurality of biological specimens in the cellular constituent data set;
   (iii) instructions for generating a receiver operating characteristic (ROC) curve for the test using the values of the function computed by the instructions for computing (i) and the indication for each organism whose cellular constituent characteristics were used in an instance of the instructions for computing (i);
   (iv) instructions for identifying one or more candidate thresholds for the test in the ROC curve; and
   (v) instructions for repeating the instructions (i) through (iv) for a different test in the plurality of tests.

In some embodiments, the one or more candidate thresholds for the test in the ROC curve are members of a convex set. In some embodiments, the convex set is the convex hull of the ROC curve. In some embodiments, there are between three and ten candidate thresholds in the convex set.

In some embodiments, the computer program product further comprises instructions for accessing a cellular constituent data set. The cellular constituent data set comprises:
a plurality of cellular constituent characteristic measurements from (i) each organism in a plurality of organisms of the species, or (ii) each biological specimen in a plurality of biological specimens from organisms of the species; and
an indication whether, for each respective organism in the plurality of organisms or for each respective organism corresponding to a biological specimen in the plurality of biological specimens, a biological feature is present or absent in the respective organism; and wherein
the instructions for scoring each candidate threshold combination comprises:
   (i) computing a model score for an organism in the plurality of organisms or for a respective organism corresponding to a biological specimen in the plurality of biological specimens using a candidate threshold combination in the plurality of candidate threshold combinations, wherein the computing comprises summing a contribution of each respective test in the model using, for each respective test, the one or more candidate thresholds for the respective test that are specified by the threshold combination;
   (ii) repeating the computing for a different organism in the plurality of organisms or for a different respective organism corresponding to a biological specimen in the plurality of biological specimens a number of times; and
   (iii) computing a receiver operating characteristic curve based upon the model scores computed in instances of the computing (i) versus the indication whether, for each respective organism in the plurality of organisms or for each respective organism corresponding to a biological specimen in the plurality of biological specimens, the biological feature is present or absent in the respective organism as specified in the cellular constituent data set; and
   (iv) assessing a goal function that is determined by the receiver operating characteristic curve.

In some embodiments, the goal function is 7*specificity+sensitivity at a point on the receiver operating characteristic curve that separates model scores that are greater than one from model scores that are less than one wherein
sensitivity=TP/(TP+FN);
specificity=TN/(TN+FP),
wherein
TP=the number of organisms considered by instances of the computing (i) that have the biological feature;
FN=the number of organisms considered by instances of the computing (i) that are falsely identified by the model as having the biological feature at the point on the receiver operating characteristic curve;
TN=the number of organisms considered by instances of the computing (i) that do not have the biological feature; and
FP=the number of organisms considered by instances of the computing (i) that are falsely identified by the model as not having the biological feature at the point on the receiver operating characteristic curve.

### 4. BRIEF DESCRIPTION OF THE DRAWINGS

FIG. 1 illustrates a computer system for constructing and/or using a classifier in accordance with one embodiment of the present invention.

FIG. 2 illustrates processing steps for constructing a classifier in accordance with one embodiment of the present invention.

FIGS. 3A and 3B illustrates processing steps for using a classifier to classify a specimen in accordance with one embodiment of the present invention.

FIG. 4 illustrates reporting steps in accordance with one embodiment of the present invention.

FIG. 5 illustrates a data structure for that stores classifiers for each of a plurality of biological classifications in accordance with one embodiment of the present invention.

FIG. 6 illustrates processing steps for constructing a classifier in accordance with another embodiment of the present invention.

FIG. 7 illustrates a receiver operating characteristic curve that is used to identify candidate positive and negative thresholds for a test in a model of the present invention.

FIG. 8 illustrates points on the convex hull of a receiver operating characteristic curve.

Like reference numerals refer to corresponding parts throughout the several views of the drawings.

### 5. DETAILED DESCRIPTION

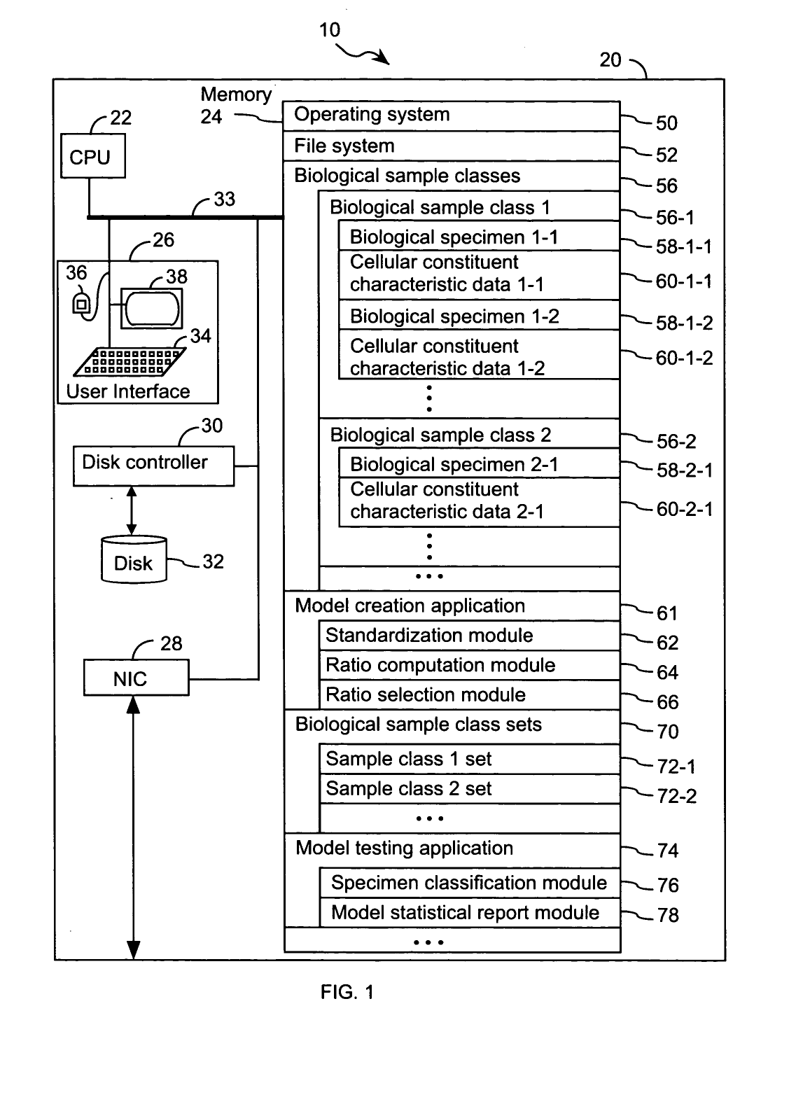

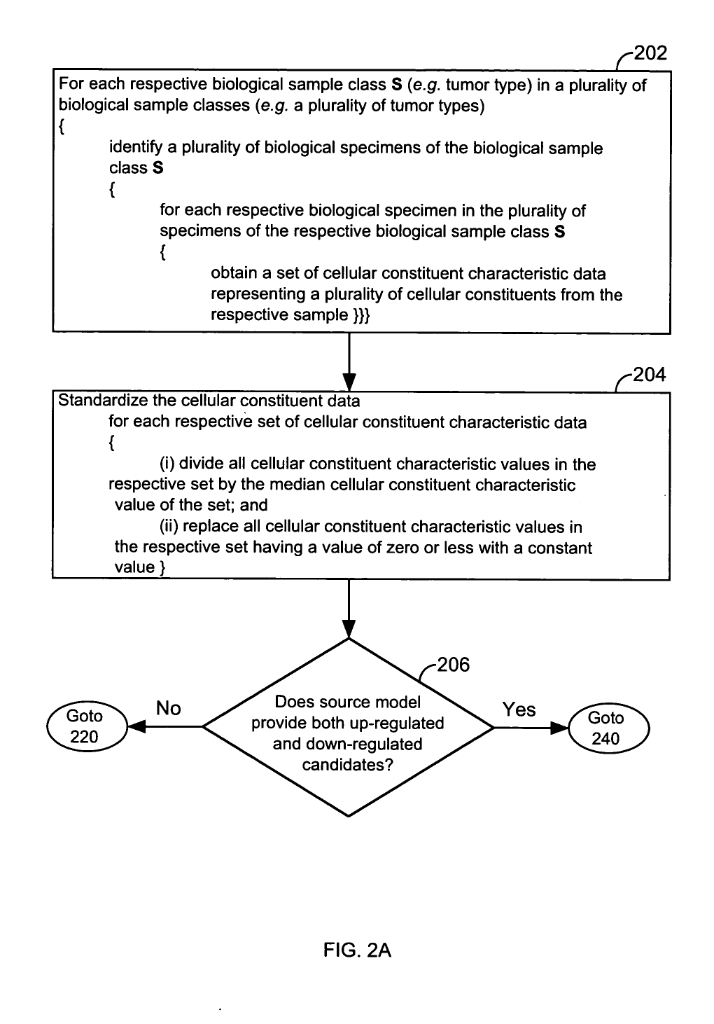

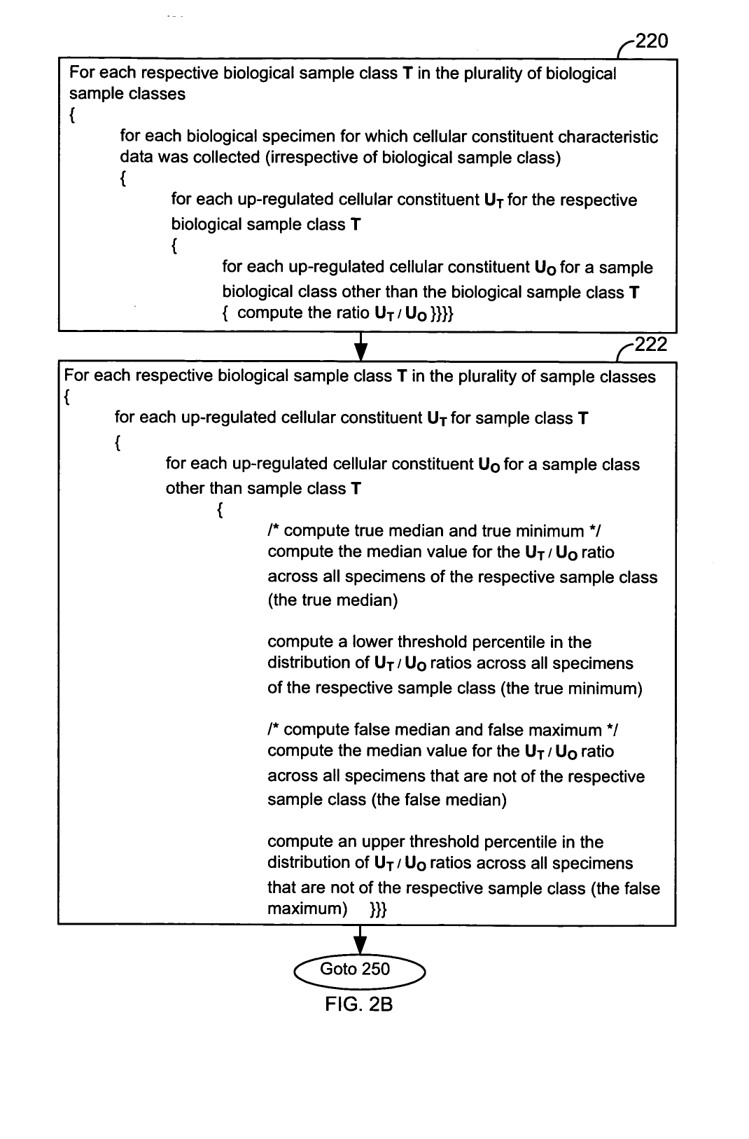

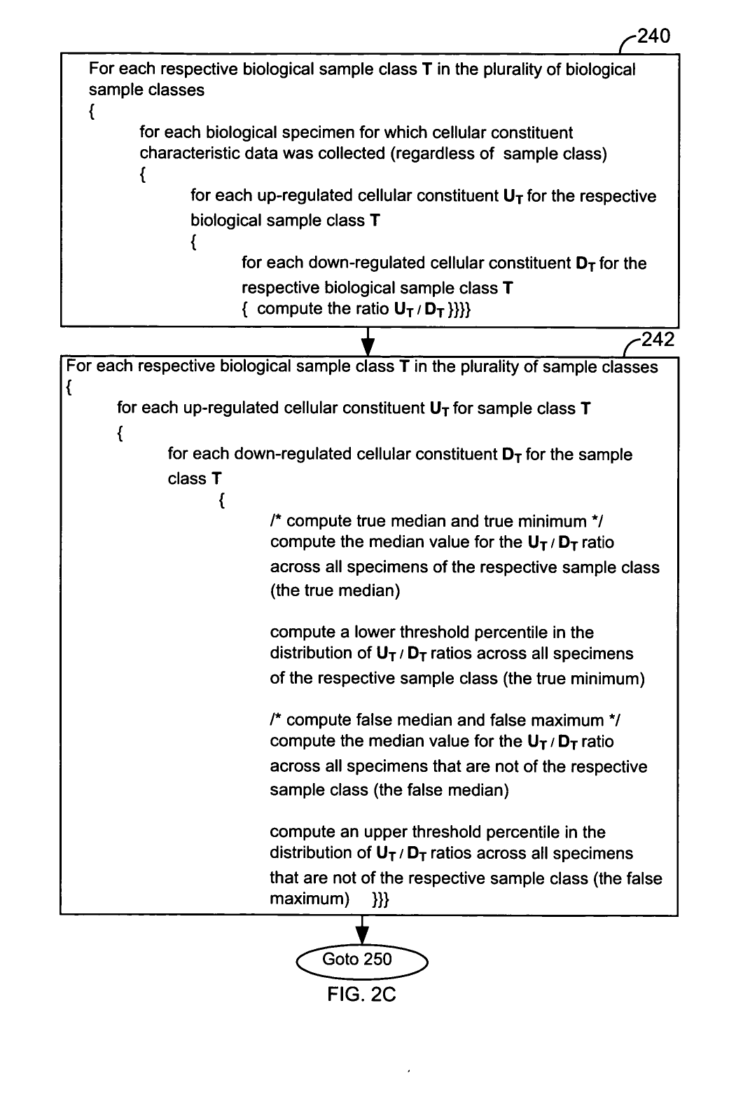

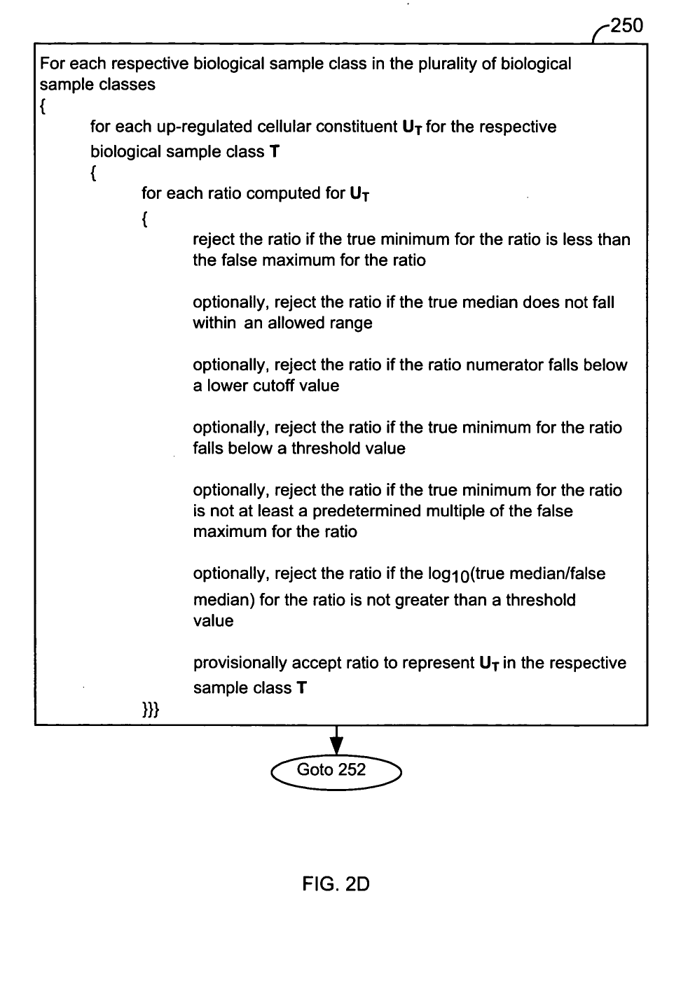

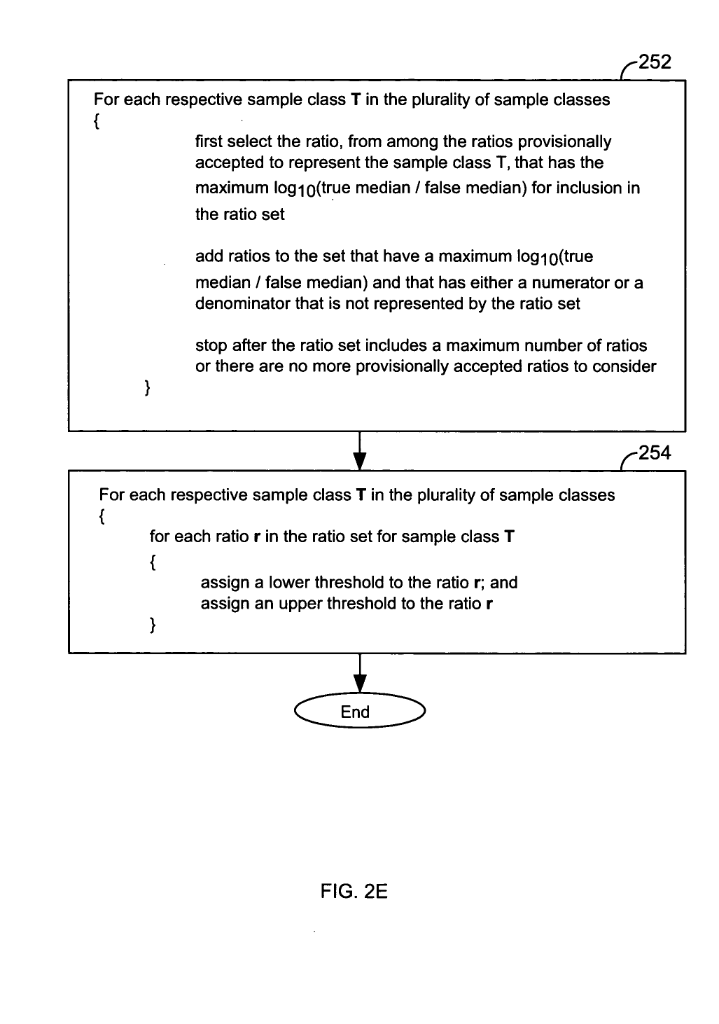

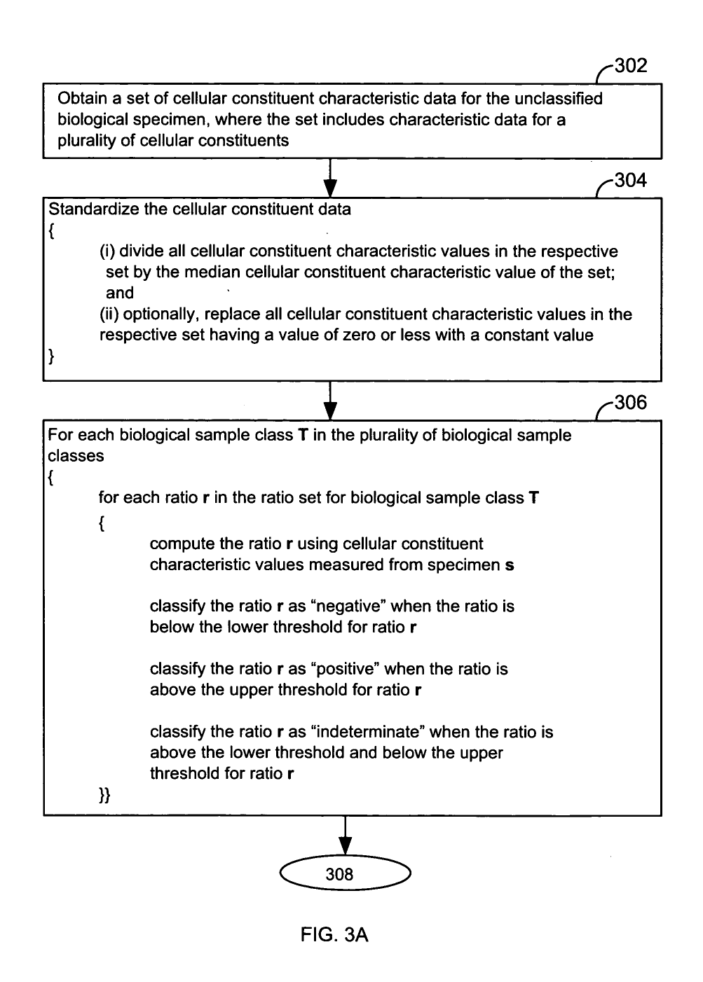

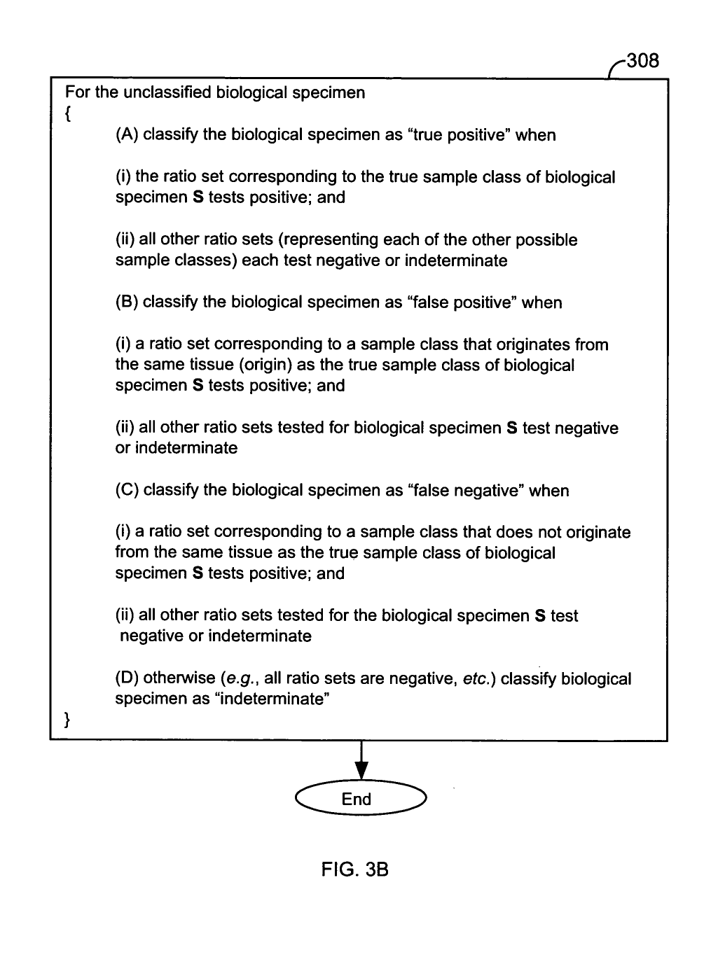

FIG. 1 illustrates a system 10 that is operated in accordance with one embodiment of the present invention. FIGS. 2A through 2E illustrate processing steps used to construct a model in accordance with one embodiment of the present invention. Using the processing steps outlined in FIGS. 3A through 3C, such models are capable of classifying a specimen into a biological class. These figures will be referenced in this section in order to disclose the advantages and features of the present invention.

System 10 comprises at least one computer 20 (FIG. 1). Computer 20 comprises standard components including a central processing unit 22, and memory 24 for storing program modules and data structures, user input/output device 26, a network interface 28 for coupling computer 20 to other computers in system 10 or other computers via a communication network (not shown), and one or more busses 33 that interconnect these components. User input/output device 26 comprises one or more user input/output components such as a mouse 36, display 38, and keyboard 34. Computer 20 further comprises a disk 32 controlled by disk controller 30. Together, memory 24 and disk 32 store program modules and data structures that are used in the present invention.

Memory 24 comprises a number of modules and data structures that are used in accordance with the present invention. It will be appreciated that, at any one time during operation of the system, a portion of the modules and/or data structures stored in memory 24 is stored in random access memory while another portion of the modules and/or data structures is stored in non-volatile storage 32. In a typical embodiment, memory 24 comprises an operating system 50. Operating system 50 comprises procedures for handling various basic system services and for performing hardware dependent tasks. Memory 24 further comprises a file system 52 for file management. In some embodiments, file system 52 is a component of operating system 50.

Now that an overview of an exemplary computer system in accordance with the present invention has been detailed, the processing steps used to create a model in accordance with one embodiment of the present invention will be described in Section 5.1, below. Section 5.3 describes the processing step used to create a model in accordance with another embodiment of the present invention. Common to each of these model creations processes is the concept of generating tests that, when polled, provide a positive, indeterminate, or negative result. Models consists of a collection of polled tests that are summed. A positive model summation indicates that an organism or biological specimen has the phenotypic feature associated with the model. A negative model summation indicates that an organism or biological specimen does not have the phenotypic feature associated with the model.

### 5.1. Model Creation

This section describes processing steps that are performed to create models in accordance with one embodiment of the present invention. In some instances, such steps are performed by model creation application 61 (FIG. 1).

Step 202.

In step 202 cellular constituent characteristic data is obtained for each respective biological sample class S in a plurality of biological sample classes to be distinguished. In particular, for each respective biological sample class S in a plurality of biological sample classes, a plurality of biological specimens of the biological sample class are identified. For each respective biological specimen B in the plurality of biological specimens of a given biological sample class, a set of cellular constituent characteristic data representing a plurality of cellular constituents from the respective biological specimen B are obtained. This obtaining is repeated for each biological sample class in the plurality of biological sample classes so that there is cellular constituent characteristic data for each biological sample class.

As an example, consider the case in which there are two biological sample classes, A and B. A plurality of biological specimens of biological sample class A are obtained. Likewise, a plurality of biological specimens of biological sample class B are obtained. For each biological specimen of biological sample class A, a cellular constituent characteristic (e.g., abundance) for a plurality of cellular constituents is measured. Further, for each biological specimen of biological sample class B, a cellular constituent characteristic (e.g., abundance) for a plurality of cellular constituents is measured. In this way, cellular constituent characteristic measurements for each biological sample class in the plurality of biological sample classes are obtained.

As used herein, biological sample classes are any distinguishable phenotype exhibited by one or more biological specimens. For example, in one application of the present invention, each biological sample class refers to an origin or primary tumor type. It has been estimated that approximately four percent of all patients diagnosed with cancer present with metastatic tumors for which the origin of the primary tumor has not been determined. See, for example, Hillen, 200, Postgrad. Med. J. 76, p. 690. On occasion, the primary site for a metastatic tumor is not clearly apparent even after pathological analysis. Thus, predicting the primary tumor site of origin for some of these cancers represent an important clinical objective. In the case of tumor of unknown primary origin, representative biological sample classes include carcinomas of the prostate, breast, colorectum, lung (adenocarcinoma and squamous cell carcinoma), liver, gastroesophagus, pancreas, ovary, kidney, and bladder/ureter, which collectively account for approximately seventy percent of all cancer-related deaths in the United States. See, for example, Greenlee et al., 2001, CA Cancer J. Clin. 51, p. 15. Section 5.3, below, describes additional examples of biological sample classes in accordance with one embodiment of the present invention.

As described above, in step 202, cellular constituent characteristic data 60 (e.g., from a gene expression study, proteomics study, etc.) is obtained for a plurality of cellular constituents from one or more members of each biological sample class 56 under study (FIG. 1, FIG. 2A). In some embodiments, the set of cellular constituent characteristic data 60 obtained from a corresponding biological specimen 58 comprises the processed microarray image for the specimen. For example, in one such embodiment, such data comprises cellular constituent abundance information for each cellular constituent represented on the array, optional background signal information, and optional associated annotation information describing the probe used for the respective cellular constituent.

In some embodiments, the cellular constituent characteristic (e.g., abundance) information is in a file format designed for Affymetrix (Santa Clara, Calif.) GeneChip probe arrays (e.g. Affymetrix chip files with a CHP extension that are generated using Affymetrix MAS5.0 software and U95A or U133 gene chips), a file format designed for Agilent (Palo Alto, Calif.) DNA microarrays, a file format designed for Amersham (Little Chalfont, England) CodeLink microarrays, the ArrayVision file format by Imaging Research (St. Catharines, Canada), the Axon (Union City, Calif.) GenePix file format, the BioDiscovery (Marina del Rey, Calif.) ImaGene file format, the Rosetta (Kirkland, Wash.) gene expression markup language (GEML) file format, a file format designed for Incyte (Palo Alto, Calif.) GEM microarrays, or a file format developed for Molecular Dynamics (Sunnyvale, Calif.) cDNA microarrays.

In some embodiments, cellular constituent characteristic measurements are transcriptional state measurements as described in Section 5.4, below. In various embodiments of the present invention, aspects of the biological state other than the transcriptional state, such as the translational state, the activity state, or mixed aspects can be measured and used as cellular constituent characteristic data. See, for example, Section 5.5, below. For instance, in some embodiments, cellular constituent characteristic data 60 is, in fact, protein levels for various proteins in the biological specimens for which cellular constituent characteristic data under study. Thus, in some embodiments, cellular constituent characteristic data comprises amounts or concentrations of the cellular constituent in tissues of the organisms under study, cellular constituent activity levels in one or more tissues of the organisms under study, the state of cellular constituent modification (e.g., phosphorylation), or other measurements relevant to the trait under study.

In one aspect of the present invention, the expression level of a gene in a biological specimen 58 is determined by measuring an amount of at least one cellular constituent that corresponds to the gene in one or more cells of the biological specimen. In one embodiment, the amount of the at least one cellular constituent that is measured comprises abundances of at least one RNA species present in one or more cells. Such abundances can be measured by a method comprising contacting a gene transcript array with RNA from one or more cells of the organism, or with cDNA derived therefrom. A gene transcript array comprises a surface with attached nucleic acids or nucleic acid mimics. The nucleic acids or nucleic acid mimics are capable of hybridizing with the RNA species or with cDNA derived from the RNA species. In one particular embodiment, the abundance of the RNA is measured by contacting a gene transcript array with the RNA from one or more cells of an organism in the plurality of organisms under study, or with nucleic acid derived from the RNA, such that the gene transcript array comprises a positionally addressable surface with attached nucleic acids or nucleic acid mimics, wherein the nucleic acids or nucleic acid mimics are capable of hybridizing with the RNA species, or with nucleic acid derived from the RNA species.

In some embodiments, cellular constituent characteristic data 60 is taken from tissues that have been associated with the corresponding biological sample class 56. For example, in the tumor of unknown primary origin, each biological specimen corresponds to a primary tumor from a known origin.

In some embodiments, cellular constituent characteristic dataset 60 (FIG. 1) comprises gene expression data for a plurality of genes (or cellular constituents that correspond to the plurality of genes). In one embodiment, the plurality of genes comprises at least five genes. In another embodiment, the plurality of genes comprises at least one hundred genes, at least one thousand genes, at least twenty thousand genes, or more than thirty thousand genes. In some embodiments, the plurality of genes comprises between five thousand and twenty thousand genes.

Step 204.

In step 204 cellular constituent data 60 is standardized. In some instances, standardization module 62 of model creation application 61 is used to perform this standardization. In some embodiments, for each respective set of cellular constituent data 60, all cellular constituent characteristic values in the set are divided by the median cellular constituent characteristic value of the set.

In the case where the source of the cellular constituent characteristic measurements is a microarray, negative cellular constituent characteristic values can be obtained when a mismatched probe measure is greater than a perfect match probe. This typically occurs when the primary gene (representing a cellular constituent) is expressed at low levels. In some representative cases, on the order of 30% of the characteristic values in a given cellular constituent characteristic dataset 60 are negative. In some embodiments of the present invention, all cellular constituent characteristic values in datasets 60 with a value of zero or less are replaced with a fixed value. In the case where the source of the cellular constituent characteristic measurements is an Affymetrix GeneChip MAS 4.0, negative cellular constituent characteristic values can be replaced with a fixed value such as 20 or 100 in some embodiments. More generally, in some embodiments, all cellular constituent characteristic values in datasets 60 with a value of zero or less can be replaced with a fixed value that is between 0.001 and 0.5 (e.g., 0.1 or 0.01) of the median cellular constituent characteristic value of the set of cellular constituent characteristic data 60. In some embodiments all cellular constituent characteristic values in datasets 60 are replaced with a transformation of the value that varies between the median and zero inversely in proportion to the absolute value of the cellular constituent characteristic value that is being replaced. In some embodiments, all or a portion of the cellular constituent characteristic values with a value less than zero are replaced with a value that is determined based on a function of the magnitude of their initial negative value. In some instances, this function is a sigmoidal function.

In preferred embodiments, the value fixed with respect to the median cellular constituent characteristic value of the set of cellular constituent characteristic data 60 represents a preferred way of handling negative values. The magnitude of such negative values is not biologically driven. Rather, it tends to represent noise. As such, a fixed value replacement is appropriate. The true biological meaning of a negative value appears to be “low express” (low abundance). In one preferred embodiment, stable results have been obtained by first standardizing the dataset 60 (dividing each cellular constituent by the median value of the dataset) and then substituting a tenth of the median value (the value 0.1) of the cellular constituent characteristic data 60 into those cellular constituents measurements that are negative or zero.

In some embodiments, standardization of cellular constituent abundances comprises dividing by the median of a subset of cellular constituents known to be particularly stable across specimens (e.g., housekeeping cellular constituents). In some embodiments, there are between five and 100 housing keeping cellular constituents, between twenty and 1000 housing keeping cellular constituents, more then two housing keeping cellular constituents, more then fifty housing keeping cellular constituents, or more than one hundred house keeping cellular constituents.

Step 206.

In step 206, a determination is made as to whether a source model provides both up-regulated and down-regulated candidates. As used herein, a source model is an indication of the cellular constituents that are up-regulated and/or down-regulated in a biological sample class 56. Source models are typically found in published references. For example, Su et al. 2001, Cancer Research 61, p. 7388 provides the names of genes that are both (i) up-regulated in specific primary tumor types and (ii) predictive of such tumor types. For example, Su et al. identified the expression of the cellular constituents listed in Table 1 with prostate tumors.

TABLE 1
Su et al. source model for prostate tumors.

| Number | Accession Name | Name | Description |
|---|---|---|---|
| 1 | NM_003656 | CAMK1 | calcium/calmodulin-dependent |
| | | | protein kinase I |
| 2 | Hs.12784 | KIAA0293 | KIAA0293 protein |
| 3 | NM_001648 | KLK3 | kallikrein 3, (prostate specific |
| | | | antigen) |
| 4 | NM_005551 | KLK2 | kallikrein 2, prostatic |
| 5 | None | TRG@ | T cell receptor gamma locus |
| | | | transcription factor similar to |
| | | | D. melanogaster |
| | | | homeodomain protein |
| 6 | NM_006562 | LBX1 | lady bird late |
| 7 | NM_016026 | LOC51109 | CGI-82 protein |
| 8 | NM_001099 | ACPP | acid phosphatase, prostate |
| 9 | NM_005551 | KLK2 | kallikrein 2, prostatic |
| 10 | None | none | Antigen \|TIGR == |
| | | | HG2261-HT2352 |
| 11 | NM_012449 | STEAP | six transmembrane epithelial |
| | | | antigen of the prostate |
| 12 | NM_001099 | ACPP | acid phosphatase, prostate |
| 13 | NM_004522 | KIF5C | kinesin family member 5C |
| 14 | None | none | Antigen \|TIGR == |
| | | | HG2261-HT2351 |
| 15 | NM_001634 | AMD1 | S-adenosylmethionine |
| | | | decarboxylase 1 |
| 16 | NM_001634 | AMD1 | S-adenosylmethionine |
| | | | decarboxylase 1 |
| 17 | None | none | Antigen \|TIGR == |
| | | | HG2261-HT2351 |
| 18 | NM_006457 | LIM | LIM protein (similar to rat |
| | | | protein kinase C-binding |
| | | | enigma) |
| 19 | NM_001648 | KLK3 | Kallikrein 3, (prostate specific |
| | | | antigen) |

The source model from Su et al. for cellular constituents associated with prostate cancer only includes genes that are up-regulated in prostate tumors. This is because Su et al. uses an initial selection criterion that selects for up-regulated genes in a given tumor type. Thus, if the models of Su et al. is used, step 206 results in a determination that the source model does not include both up-regulated and down-regulated cellular constituent candidates (206-No) and control passes to step 220 of FIG. 2B. If, on the other hand the source model includes cellular constituents that are both up-regulated and down-regulated in a given biological sample class (step 206-Yes), control passes to step 240 of FIG. 2C. In some embodiments, control passes to step 220 regardless of whether or not the source model includes both up-regulated and down-regulated cellular constituent candidates.

Step 220.

In step 220 a plurality of test ratios is calculated for a biological sample class 56. In some embodiments these ratios are computed using ratio computation model 64. The numerator and denominator of any given ratio in the plurality of test ratios is computed using cellular constituent characteristic data from a single biological specimen. In some embodiments, ratio numerators are determined by a characteristic (e.g., abundance) of a first cellular constituent that is up-regulated or down-regulated in the biological sample class 56. In some embodiments, a cellular constituent is up-regulated in the biological sample class when the characteristic of the cellular constituent in biological specimens of the biological sample class is greater than the characteristic of at least sixty percent, at least seventy percent, at least eighty percent or at least ninety percent of the cellular constituents in biological specimens of the biological sample class for which cellular constituent characteristic measurements have been made. In some embodiments a cellular constituent is down-regulated in a biological sample class when the characteristic of the cellular constituent in biological specimens of the biological sample class is less than the characteristic of at least forty percent, at least thirty percent, at least twenty percent, or at least ten percent of the cellular constituents in biological specimens of the biological sample class for which cellular constituent characteristic measurements have been made.

In some embodiments, ratio denominators are determined by a characteristic (e.g., abundance) of a second cellular constituent. In some embodiments, the first cellular constituent and the second cellular constituent are each a nucleic acid or a ribonucleic acid and the characteristic of the first cellular constituent and the characteristic of the second cellular constituent in each biological specimen is obtained by measuring a transcriptional state of all or a portion of the first cellular constituent and the second cellular constituent. In some embodiments, the first cellular constituent and the second cellular constituent are each all or a fragment of an mRNA, a cRNA or a cDNA. In some embodiments, the first cellular constituent and the second cellular constituent are each proteins and the characteristic of the first cellular constituent and the characteristic of the second cellular constituent is obtained by measuring a translational state of all or a portion of the first cellular constituent and the second cellular constituent. In some embodiments, a characteristic (e.g., abundance) of the first cellular constituent and a characteristic of the second cellular constituent is determined using isotope-coded affinity tagging followed by tandem mass spectrometry analysis. In still other embodiments, the characteristic of the first cellular constituent and the characteristic of the second cellular constituent is determined by measuring an activity or a post-translational modification of the first cellular constituent and the second cellular constituent.

More than one biological sample class 56 in the plurality of biological sample classes is represented in the plurality of test ratios. Step 220 is best explained using an example. Consider the case in which there are two biological sample classes 56. The first biological sample class is prostate tumors and the source model for this first biological sample class are the genes listed in Table 1 above. A plurality of ratios are computed for this first biological sample class. More than one sample class is represented in this plurality of test ratios. Thus, biological specimens that belong to the first biological sample class and biological specimens that belong to the second biological sample class are used to compute the plurality of test ratios. Consider the case in which there is cellular constituent characteristic data from ten biological specimens of the first sample class (prostate tumors) and ten biological specimens from the second sample class for a total of twenty specimens. The following calculations are made:

```
for each biological specimen for which cellular constituent data was
collected (for each of the 10 prostate tumors and the ten biological
specimens from the second class)
{
  for each up-regulated cellular constituent U_{T} for the respective
  biological sample class T (for each of the cellular
  constituents in table 1)
  {
    for each up-regulated cellular constituent U_{O} for a biological sample
    class other than biological sample class T (for each up-regulated
    cellular constituent in sample class B)
    {
      compute the ratio U_{T}/U_{O}}}}.
```

In these calculations, each numerator represents a cellular constituent that is up-regulated in the biological sample class for which the ratio are calculated. In other embodiments, each numerator represents a cellular constituent that is down-regulated in the biological sample class for which the ratio are calculated. In the calculations described above, the denominator represents cellular constituents that are up-regulated in biological sample classes other than the biological sample class that represents prostate tumors. It will be appreciated that, in this example, if every possible combination of ratios is computed for every possible biological sample, a total of
A×D×N
test ratios will be calculated, where
A is the number of up-regulated cellular constituents in the biological sample class S (e.g., A is 19 because there are 19 genes in Table 1 above);
D is the total number of up-regulated cellular constituents in the plurality of biological sample classes with the exception of the biological sample class S; and
N is the number of biological specimens used in the computation of the plurality of test ratios (N is twenty because there are 10 biological specimens that are prostate tumors and 10 biological specimens that are not prostate tumors).

In this example, consider the case in which the second biological sample class is bladder tumors. Su et al., 2001, Cancer Research 61, p. 7388 identified the cellular constituents listed Table 2 as those cellular constituents that were both (i) up-regulated in bladder tumors and (ii) indicative of bladder tumors.

TABLE 2
Su et al. source model for bladder tumors.

| Number | Accession Name | Name | Description |
|---|---|---|---|
| 1 | NM_006760 | UPK2 | uroplakin 2 |
| 2 | NM_006788 | RALBP1 | ralA binding protein 1 |
| 3 | NM_003087 | SNCG | synuclein, gamma (breast |
| | | | cancer-specific protein 1) |
| 4 | NM_001068 | TOP2B | topoisomerase (DNA) II beta |
| | | | (180 kD) |
| 5 | NM_003282 | TNNI2 | troponin I, skeletal, fast |
| 6 | None | MYCL1 | v-myc avian myelocytomatosis |
| | | | viral oncogene homolog 1, lung |
| | | | carcinoma derived |
| 7 | NM_005037 | PPARG | peroxisome proliferative |
| | | | activated receptor, gamma |
| 8 | None | COL4A6 | collagen, type IV, alpha 6 |
| 9 | NM_006829 | APM2 | adipose specific 2 |
| 10 | NM_014452 | DR6 | death receptor 6 |
| 11 | NM_001190 | BCAT2 | branched chain aminotransferase |
| | | | 2, mitochondrial |
| 12 | Nm_006952 | UPK1B | uroplakin 1B |

In this case, there will be a total of
A×D×N test ratios
computed for the prostate tumor biological sample class,
where,
   A is the nineteen up-regulated cellular constituents in prostate tumors;
   D is the twelve up-regulated cellular constituents in bladder tumors; and
   N is the 10 biological specimens that are prostate tumors plus the 10 biological specimens that are bladder tumors. Thus, a total of 4560 ratios are computed for the prostate tumor biological sample class.

The present invention is not limited to instances where there are only two biological sample classes. Consider an extension of the example in which cellular constituent characteristic data for ten biological specimens belonging to a third biological sample class 56, breast cancer, is available. Su et al., 2001, Cancer Research 61, p. 7388 identified the cellular constituents listed Table 3 as those cellular constituents that were both (i) up-regulated in breast cancer and (ii) indicative of breast cancer.

TABLE 3
Su et al. source model for breast tumors.

| | Accession | | |
|---|---|---|---|
| Number | Name | Name | Description |
| 1 | NM_005853 | IRX5 | iroquois homeobox protein 5 |
| 2 | NM_004064 | CDKN1B | cyclin-dependent kinase |
| | | | inhibitor 1B (p27, Kip1) |
| 3 | None | FLJ13612 | hypothetical protein FLJ13612 |
| 4 | NM_002411 | MGB1 | mammaglobin 1 |
| 5 | Hs.288467 | None | Homo sapiens cDNA FLJ12280 fis, |
| | | | clone MAMMA1001744 |
| 6 | NM_005264 | GFRA1 | GDNF family receptor alpha 1 |
| 7 | Hs.209607 | None | Homo sapiens endogenous |
| | | | retrovirus HERV-K104 long terminal |
| | | | repeat, complete sequence; and Gag |
| | | | protein (gag) and envelope protein |
| | | | (env) genes, complete cds |
| 8 | NM_004460 | FAP | fibroblast activation protein, alpha |
| 9 | NM_024113 | COMP | cartilage oligomeric matrix protein |
| | | | (pseudoachondroplasia, epiphyseal |
| | | | dysplasia 1, multiple) |
| 10 | NM_024830 | FLJ12443 | hypothetical protein FLJ12443 |
| 11 | None | C18ORF1 | chromosome 18 open reading frame 1 |
| 12 | NM_000095 | COMP | cartilage oligomeric matrix protein |
| | | | (pseudoachondroplasia, epiphyseal |
| | | | dysplasia 1, multiple) |

In this case, there will be a total of
A×D×N test ratios
computed for the prostate tumor biological sample class where,
A is the nineteen up-regulated cellular constituents in prostate tumors;
D is the twelve up-regulated cellular constituents in bladder tumors plus the twelve up-regulated cellular constituents in breast cancers; and
N is the 10 biological specimens that are prostate tumors plus the 10 biological specimens that are bladder tumors plus the ten biological specimens that are breast cancers. Thus, a total of 13,680 ratios can be computed for the prostate tumor biological sample class in this example. An example of a one of the 13,680 ratios that is computed is:
   [Characteristic of CAMK1]/[Characteristic of UPK2] in a biological specimen B from any of the three biological sample classes considered
where,
[Characteristic of CAMK1] is the characteristic of the cellular constituent CAMK1 in the biological specimen B, and
[Characteristic of UPK2] is the characteristic of the cellular constituent UPK2 in the biological specimen B.

Step 222.

In step 220, a large number of ratios are computed for each biological sample class 56 under consideration. Each cellular constituent pair defined by each of these calculated ratios (where the cellular constituent pair is the cellular constituent in the numerator and the cellular constituent in the denominator) is a potential candidate for a final biological sample set of cellular constituent pairs 72 that represents a corresponding biological sample class 56. In step 222, information about the ratios calculated in step 220 is derived so that certain cellular constituent pairs (and their corresponding ratio) can be removed from consideration for the final biological sample class set 72 (FIG. 1) that will represent one of the biological sample classes 56 in the plurality of biological sample classes. This process is repeated for each biological sample class 56 under consideration. In some embodiments, step 222 is performed by ratio selection module 66 of model creation application 61 (FIG. 1).

Some embodiments of step 222 comprise calculating information that is used to determine a set of cellular constituent pairs 72 for a biological sample class 56 in the plurality of biological sample classes from the corresponding plurality of test ratios for the biological sample class 56 computed in step 220, thereby constructing a classifier for the biological sample class. In the example presented above, where prostate, bladder, and breast tumor biological specimens were considered, the plurality of test ratios for the prostate biological sample class is the 13,680 ratios computed using cellular constituent data from tables 1 through 3.

In step 222, the true median, true minimum, false median, and false maximum for a ratio r calculated in step 220 is obtained. To understand how these statistics are obtained for a given ratio r, it must first be understood how the plurality of ratios calculated in step 220 are handled in step 222. In step 222, ratios that have the same numerator and same denominator are considered a set. For example, all ratios of the type
[Characteristic of CAMK1]/[Characteristic of UPK2]
where the characteristic data for the ratio is collected from any of the biological specimens tested, are considered a single set. Thus, in this set, there will be a first ratio that is defined by
[Characteristic of CAMK1]/[Characteristic of UPK2]
that is from biological specimen 1, a second ratio that is defined by
[Characteristic of CAMK1]/[Characteristic of UPK2]
from biological specimen 2, and so forth. This set of ratios is divided into two subsets (i) a first subset that represents those ratios that are computed using characteristic data from specimens of the biological sample class 56 under consideration (e.g., prostate tumors) and (ii) a second subset that represents those ratios that are computed using characteristic data from biological specimens belonging to biological sample classes other than the biological sample class 56 under consideration (e.g., bladder tumors and breast tumors). The first subset of ratios forms a first distribution (the true distribution) and the second subset of ratios forms a second distribution (the false distribution).

The true minimum for the given ratio r is a lower threshold percentile in the first distribution (of the first subset of the set of test ratios). The true median is the median value of the first distribution. The false median is the median value of the second distribution and the false maximum is an upper threshold percentile of the second distribution. In some embodiments, the lower threshold percentile is between the tenth and thirtieth percentile of the distribution of the first subset of test ratios and the upper threshold percentile is between the seventieth and ninety-fifth percentile of the distribution of the second subset of test ratios. In some embodiments, the lower threshold percentile is between the tenth and thirtieth percentile of the distribution of the first subset and the upper threshold percentile is between the seventieth and ninety-fifth percentile of the distribution of the second subset.

Step 240.

Step 240 is reached from step 206 in cases where the source model includes both up-regulated and down-regulated candidates. Step 240 is similar to step 220 in that a large number of ratios are computed for each biological sample class 56 under consideration. In some embodiments these ratios are computed using ratio computation model 64. The numerator and denominator of any given ratio in the plurality of test ratios is computed using cellular constituent characteristic data from a single biological specimen. In typical instances of step 240, ratio numerators are determined by a characteristic (abundance) of a first cellular constituent that is up-regulated in the biological sample class 56 while ratio denominators are determined by a characteristic of a second cellular constituent that is down-regulated in the biological sample class 56. Of course, the reciprocal arrangement, where ratio numerators represent down-regulated cellular constituents and ratio denominators represent up-regulated cellular constituents can also be computed in step 240. However, for simplicity of presentation, the former case (ratio numerators representing up-regulated cellular constituents) will be discussed. As is in the case of step 220, more than one biological sample class 56, in the plurality of biological sample classes, is represented in the plurality of test ratios calculated for each biological sample class 56.

Like step 220, step 240 is best explained by example. Consider the case in which there are two biological sample classes 56. The first biological sample class is prostate tumors and the source model for this first biological sample class includes the up-regulated genes listed in Table 1 above as well as a plurality of down-regulated genes in prostate tumors (not disclosed). A plurality of ratios are computed for this first biological sample class. More than one sample class is represented in this plurality of test ratios. Thus, biological specimens that belong to the first biological sample class and biological specimens that belong to the second biological sample class are used to compute the plurality of test ratios. Consider the case in which there is cellular constituent characteristic data from ten biological specimens of the first sample class (prostate tumors) and ten biological specimens from the second sample class for a total of twenty specimens. The following calculations are made:

```
for each biological specimen for which cellular constituent data was
collected (for each of the 10 prostate tumors and the ten biological
specimens from the second class
{
  for each up-regulated cellular constituent U_{T} for the respective
  biological sample class T (for each of the cellular
  constituents in table 1)
  {
    for each down-regulated cellular constituent D_{T} for the respective
    biological sample class T (for each down-regulated cellular
    constituent in prostate tumors)
    {
      compute the ratio U_{T}/DT}}}.
```

It will be appreciated that if every possible UT and DT is combined into a ratio, the total number of ratios computed for prostate tumors will be:
A×B×N test ratios
where,
A is the number of up-regulated cellular constituents in the biological sample class S (e.g., A is 19 because there are 19 genes in Table 1 above);
B is the total number of down-regulated cellular constituents in the biological sample class S; and
N is the number of biological specimens used in the computation of the plurality of test ratios (N is twenty because there are 10 biological specimens that are prostate tumors and 10 biological specimens that are not prostate tumors).

Step 242.

In step 240, a large number of ratios are computed for each biological sample class 56 under consideration. Each of these calculated ratios is a potential candidate for a final biological sample set 72 that represents a corresponding biological sample class 56. In step 242, information about the ratios calculated in step 240 is derived so that certain ratios (e.g., the cellular constituent pairs determined by such ratios) can be removed from consideration for the final biological sample class set 72 (FIG. 1) that will represent one of the biological sample classes 56 in the plurality of biological sample classes. This process is repeated for each biological sample class 56 under consideration. In some embodiments, step 242 is performed by ratio selection module 66 of model creation application 61 (FIG. 1). Step 242 largely corresponds to step 222 (FIG. 2).

Some embodiments of step 242 comprise calculating information that is used to determine a set 72 for a biological sample class 56 in the plurality of biological sample classes from the corresponding plurality of test ratios for the biological sample class 56 computed in step 220, thereby constructing a classifier for the biological sample class. In step 242, the true median, true minimum, false median, and false maximum for a ratio r calculated in step 240 is obtained. To understand how these statistics are obtained for a given ratio r, it must first be understood how the plurality of ratios calculated in step 240 are handled in step 242. In step 242, ratios that have the same numerator and the same denominator are considered a set. For example, all ratios of the type
[Characteristic of CAMK1]/[Characteristic of a given gene that is down-regulated in prostate tumors]
where the characteristic data for the ratio is collected from any of the biological specimens tested, are considered a single set. Thus, in this set, there will be a first ratio defined by
[Characteristic of CAMK1]/[Characteristic of a given gene that is down-regulated in prostate tumors]
that is from biological specimen 1, a second ratio defined by
[Characteristic of CAMK1]/[Characteristic of a given gene that is down-regulated in prostate tumors]
from biological specimen 2, and so forth. This set of ratios is divided into two subsets (i) a first subset that represents those ratios that are computed using characteristic data from specimens of the biological sample class 56 under consideration (e.g., prostate tumors) and (ii) a second subset that represents those ratios that are computed using characteristic data from biological specimens belonging to biological sample classes other than the biological sample class 56 under consideration (e.g., bladder tumors). The first subset of ratios forms a first distribution (the true distribution) and the second subset of ratios forms a second distribution (the false distribution). Then, the true minimum, true median, false median, and false maximum are defined based on the true distribution and the false distribution in the same way that they are defined in step 222, above.

Step 250.

In FIG. 2, either steps 220 and 222 or steps 240 and 242 are performed based on the results of the decision made as step 206. Step 250 closes this branch. In other words, step 250 is performed regardless of the outcome of step 206. In step 250, select ratios (i.e. the cellular constituent pairs determined by such ratios where the numerator is the first cellular constituent in such pairs and the denominator is the second cellular constituent in such pairs) calculated for a biological sample class 56 in step 220 (or step 240) are rejected based on one or more criteria. The rejection criteria make use of the fact that the cellular constituent characteristic data has been standardized in step 204. In some embodiments, a ratio is rejected when the true minimum for the ratio is less than the false maximum. To illustrate, consider the case in which the ratio
[Characteristic of CAMK1]/[Characteristic of UPK2]
is being assessed in order to determine whether to reject the cellular constituent pair (CAMK1, UPK2). This ratio, from every biological specimen, regardless of which biological sample class the specimens belong to, is collected to form a set of ratios. The set of ratios is divided into a first and second subset. Each ratio in the first subset is the ratio CAMK1/UPK2 from a prostate tumor. Each ratio in the second subset is the ratio CAMK1/UPK2 from a bladder or breast tumor. The first and second subsets of ratios respectively form first and second distributions. When the true minimum for the ratio CAMK1/UPK2 is less than the false maximum for the ratio, the cellular constituent pair (CAMK1, UPK2) is discarded from consideration for use as a classifier for prostate tumors.

In some embodiments the true minimum is a lower threshold percentile of the first distribution. In some instances, this lower threshold percentile is between the tenth and thirtieth percentile of the first distribution (the distribution of the first subset of test ratios). Further, in some embodiments, the false maximum is an upper threshold percentile that is between the seventieth and ninety-fifth percentile of the second distribution (the distribution of the second subset of test ratios). In some instances, the lower threshold percentile of the first distribution is between the tenth and thirtieth percentile of the first distribution and the upper threshold percentile of the second distribution is between the seventieth and ninety-fifth percentile of the second distribution.

In addition to the requirement that the true minimum for a ratio be greater than the false maximum for the ratio, additional optional selection criteria can be implemented in order to identify ratios that discriminate between the biological sample classes 56 under consideration. For example, in some embodiments, a ratio is rejected if the true median for the ratio does not fall within an allowed range. In other words, in order to be considered for the final set 72 for a biological sample class 56, a given ratio r for the biological sample class 56 must have a true median that is greater than a lower allowed value and less than a higher allowed value, where the true median for the given ratio r is the median value of the first subset of test ratios selected from the plurality of test ratios calculated for the biological sample class 56 that the given ratio r represents. While the lower allowed value and the higher allowed value will vary depending on the way cellular constituent characteristic data is measured, in some embodiments, the lower allowed value is 25 and the higher allowed value is 2000. In other embodiments, the lower allowed value is 50 and the higher allowed value is 1000.

In some embodiments, cellular constituent pair is rejected when the numerator of the ratio corresponding to the cellular constituent pair numerator falls below a lower cutoff value. This type of rejection makes use of the fact that cellular constituent characteristic values have been standardized. For example, in some instances, the lower cutoff value (lower allowed value) is two. This ensures that the numerator, which in such embodiments represents an up-regulated cellular constituent, is in fact up-regulated. Because cellular constituent characteristic data has been standardized, a value of two represents twice the median cellular constituent characteristic of the plurality of cellular constituents from the biological specimen 56 from which ratio characteristics were measured. In some embodiments, the cellular constituent pair for a ratio is rejected when the true minimum for the ratio is less than a threshold value, such as one. This ensures that the numerator, which in such embodiments represents an up-regulated cellular constituent, is in fact up-regulated.

In some embodiments, a ratio is rejected when the true minimum for the given ratio r is not at least a predetermined multiple (e.g. 1.2) of the false maximum for the ratio. This criterion ensures that only those ratios in which the true distribution clearly differentiates from the false distribution are selected for use in a classifier.

Another criterion that can be used to reject ratios makes use of the log₁₀(true median/false median) for the a given ratio. For instance, in some embodiments, a ratio is rejected when the log₁₀(true median/false median) of the ratio is not greater than a threshold value (e.g., not greater than 2, not greater than 3, not greater than 4, etc.).

Step 252.

In step 250, one or more criteria were used to eliminate from consideration ratios that had been calculated, based on cellular constituent pairs, for each biological sample class 56 under consideration. In step 252, ratios (i.e., the cellular constituent pairs that correspond to such ratios) are selected from the pool of remaining ratios in order to build a set 72 for each biological sample class 72 under consideration.

In some embodiments, the cellular constituent pair corresponding to the ratio calculated for a given biological sample class 56 that has the largest log₁₀(true median/false median) is selected for inclusion in the biological sample class set 72 corresponding to the biological sample class. Then the cellular constituent pair corresponding to the ratio that has the next highest log₁₀(true median/false median) and that has a cellular constituent in either the numerator or denominator that is not represented in the numerator or denominator of any ratio already in the set 72 is selected for inclusion in the biological sample class set 72. This process continues, where no cellular constituent pair is added to set 72 unless it corresponds to a ratio that has a cellular constituent in either the numerator or denominator that is not present in the numerator or denominator of any ratio represented by cellular constituent pairs already in set 72, until a desired number of cellular constituent pairs for the biological sample class 56 have been included in the set 72. In some embodiments each set 72 has between two and one thousand cellular constituent pairs (defining between two and one thousand cellular constituent pairs). In some embodiments, each set 72 has between two and one hundred cellular constituent pairs. In a preferred embodiment, each set 72 comprises between three and five cellular constituent pairs representing between three and five ratios.

Step 254.

In step 254, for each respective biological sample class 56 considered, for each cellular constituent pair (ratio) in the set 72 corresponding to the respective biological sample class, the lower threshold to the ratio defined by the cellular constituent pair (e.g., the false maximum) and the upper threshold (e.g., the true minimum) are associated with the ratio.

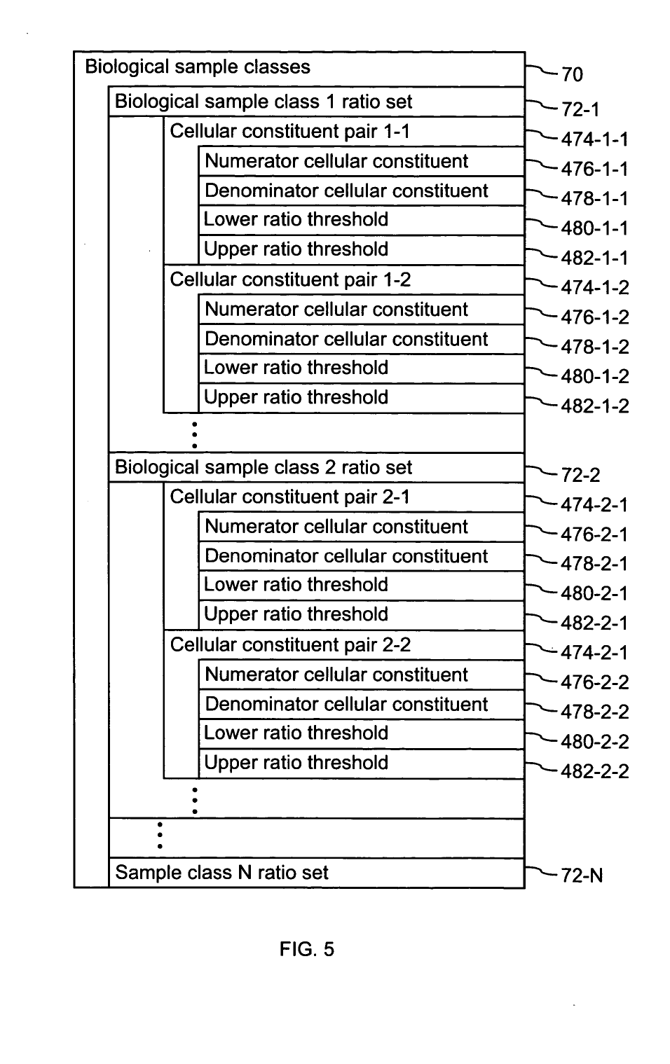

FIG. 5 illustrates the results of the processing steps illustrated in FIG. 2. FIG. 5 illustrates a data structure 70 that represents a plurality of biological sample classes 56. For each biological sample class 56 there is a corresponding sample class set 72. Each sample class set 72 includes cellular constituent pairs 474. Each cellular constituent pair 474 includes a numerator cellular constituent 476. In typical embodiments, a numerator cellular constituent 476 for a cellular constituent pair 474 in the set 72 of a given biological sample class 56 is up-regulated in the given biological sample class 56 relative to another biological sample class. However, in alternative embodiments, the numerator cellular constituent 476 is down-regulated in the given biological sample class 56 relative to another biological sample class.

Each cellular constituent pair 474 includes a denominator cellular constituent 478. In some embodiments, each denominator cellular constituent 478 in the set 72 of a given biological sample class 56 is down-regulated in the biological sample class relative to another biological sample class. In some embodiments, each denominator cellular constituent 478 in the set 72 of a given biological sample class 56 is up-regulated in one or more biological sample classes relative to the biological sample class represented by the set 72.

In typical embodiments, at least one of the numerator 476 and denominator 478 of each cellular constituent pair 474 in a given set 72 is not found in the numerator 476 or denominator 478 of any other cellular constituent pair in the given set 72. In other words, each cellular constituent pair has at least one unique cellular constituent. As further illustrated in FIG. 5, each cellular constituent pair 474 includes a lower ratio threshold 480 and an upper ratio threshold 482. These threshold are the respectively the false maximum and true minimum that have been computed for the ratio defined the cellular constituent pair.

Each biological sample class set illustrated in FIG. 5 represents a highly advantageous classifier in accordance with the present invention. As will be described in Section 5.2, below, these classifiers can be used to determine which biological sample class 72 a particular biological specimen belongs.

### 5.2. Model Application

Methods for generating classifiers that comprise a different set of cellular constituent pairs associated with each biological sample class 56 in a plurality of biological sample classes 56 have been described in Section 5.1, above. In this section, methods for using such sets of cellular constituent pairs to determine the biological classification of a previously unclassified biological sample are described in conjunction with FIG. 3. In some embodiments, the steps illustrated in FIG. 3 are performed using model testing application 74 (FIG. 1).

Step 302.

In step 302, a set of cellular constituent characteristic data is obtained for the unclassified biological specimen. This set of cellular constituent characteristic data represents a plurality of cellular constituents from the unclassified biological specimen. In some embodiments, the set of cellular constituent characteristic data obtained in step 302 comprises the processed microarray image for the specimen. In some embodiments, cellular constituent characteristic measurements taken in step 302 are transcriptional state measurements as described in Section 5.4, below. In some embodiments of step 302, aspects of the biological state other than the transcriptional state, such as the translational state, the activity state, or mixed aspects can be measured and used as cellular constituent characteristic data. See, for example, Section 5.5, below. For instance, in some embodiments, cellular constituent characteristic data measured in step 302 is, in fact, protein levels for various proteins in the biological specimens for which cellular constituent characteristic data under study. Thus, in some embodiments, cellular constituent characteristic data measured in step 302 comprises amounts or concentrations of the cellular constituent in tissues of the organisms under study, cellular constituent activity levels in one or more tissues of the organisms under study, the state of cellular constituent modification (e.g., phosphorylation), or other measurements relevant to the trait under study.

Step 304.

In some embodiments of step 304, the set of cellular constituent characteristic data measured for the unclassified biological specimen is standardized by dividing all cellular constituent characteristic values in the set by the median cellular constituent characteristic value of the set. In other embodiments of step 304, the set of cellular constituent characteristic data measured for the unclassified biological specimen is divided by the average of the 25^{th} and 75^{th} percentile of the set.

As described in step 202 above, in the case where the source of the cellular constituent characteristic measurements is a microarray, negative cellular constituent characteristic values can be obtained. In some embodiments of step 304, all cellular constituent characteristic values in the set having a value of zero or less are replaced with a fixed value. In the case where the source of the cellular constituent characteristic measurements is an Affymetrix GeneChip MAS 4.0, negative cellular constituent characteristic values are replaced with a fixed value such as 20 or 100 in some embodiments. More generally, in some embodiments all cellular constituent characteristic values with a value of zero or less are replaced with a fixed value that is between 0.001 and 0.5 (e.g., 0.1 or 0.01) of the median cellular constituent characteristic value of the set. In some embodiments all cellular constituent characteristic values are replaced with a transformation of the value that varies between the median and zero inversely in proportion to the absolute value of the cellular constituent characteristic value that is being replaced. In some embodiments, all or a portion of the cellular constituent characteristic values with a value less than zero are replaced with a value that is determined based on a function of the magnitude of their initial negative value. In some instances, this function is a sigmoidal function. In one embodiment, the set obtained in step 202 is first standardized (by dividing each cellular constituent by the median value of the set) and then values in the set with zero or negative values are substituted with a value that is a tenth of the median value (the value 0.1) of the set.

Step 306.

In typical embodiments, the unclassified biological specimen could belong to any one of a number of biological sample classes 56. As a result of the steps described in Section 5.1 above, each biological sample class is associated with a different set 72. In step 306 the ratios defined by each such set are computed using cellular standardized cellular constituent characteristic values from the biological sample. Logically, this computation can be expressed as:

```
for each respective biological sample class T (56) in a plurality
of biological sample classes
{
  for each ratio r defined by the set (72) for the biological sample class T
  {
    compute the ratio r using cellular constituent characteristic values
      measured from the unclassified biological specimen
  }}
```

In this way, each possible ratio needed for each of the sets of the candidate biological sample classes is computed.

In addition to computing ratios, step 306 classifies ratios. As described in Section 5.1 above, and as illustrated in FIG. 5, an upper ratio threshold and a lower ratio threshold is assigned to each ratio in sets 72. In step 306, each ratio computed based on standardized cellular constituent characteristic values from the unclassified biological specimen is characterized based upon these upper and low ratio thresholds as follows:

```
for each respective biological sample class T (56) in a plurality
of biological sample classes
{
  for each ratio r in the set (72) for the biological sample class T
computed using cellular constituent characteristic values measured from
the unclassified biological specimen
  {
    (i) identify the ratio as “negative” when the value of the ratio is
    below the lower threshold value for the ratio;
    (ii) identify the ratio as “positive” when the value of the ratio is
    above the upper threshold value for the ratio; and
    (iii) identify the ratio as “indeterminate” when the value of the ratio is
    above the lower threshold value and below the upper threshold
    value for the ratio
  }}
```

Such assignments are based on the assumption that the numerator of each ratio is up-regulated. In other embodiments, this is not the case and the numerator of each ratio is down-regulated. In such embodiments, each ratio is assigned in the reverse manner (e.g., the ratio is identified as “positive” when the value is above the lower threshold value for the ratio). However, for the sake of clear illustration of one embodiment of the invention, the case in which the numerator in a ratio represents an up-regulated cellular constituent in associated sample class is described. Those of skill in the art, upon reviewing this embodiment of the invention as disclosed herein, will appreciate the various permutations and variants of the embodiment and all such embodiments are within the scope of the present invention.

An example will facilitate the understanding of step 306. Consider the case in which there is an unknown biological specimen for which cellular constituent characteristic data has been measured and standardized in accordance with steps 302 and 304. In step 306, ratios of these characteristics (e.g., abundance) are computed. Specifically, ratios of cellular constituent characteristics designated in the sets 72 for candidate biological sample classes 56 are computed. In one such computation, the ratio [A₁]/[B₁] is computed, where [A₁] and [B₁] are respectively the characteristics of the cellular constituents A₁ and B₁ in the unclassified biological specimen. The set 72 comprising the ratio [A₁]/[B₁] includes a corresponding lower ratio threshold 480 and upper ratio threshold 482. These values are used to characterize the ratio [A₁]/[B₁].

In one instance [A₁] is 1000, [B,] is 100, the lower ratio threshold is 0.8 and the upper ratio threshold is 5. In such an instance, the ratio [A₁]/[B₁] has the value 10. Because the ratio is greater than the upper ratio threshold, the ratio is characterized as “positive.”

In another instance [A₁] is 70, [B₁] is 100, the lower ratio threshold is 0.8 and the upper ratio threshold is 5. In such an instance, the ratio [A₁]/[B₁] has the value 0.7. Because the ratio is less than the lower ratio threshold, the ratio is characterized as “negative.”

In still another instance [A₁] is 120, [B₁] is 100, the lower ratio threshold is 0.8 and the upper ratio threshold is 5. In such an instance, the ratio [A₁]/[B₁] has the value 1.2. Because the ratio is greater than the lower ratio threshold but less than the upper ratio threshold, the ratio is characterized as “indeterminate.”

Step 308.

In step 308 the unclassified biological sample is classified based on the ratio calculations made in step 306. This is done by characterizing sets 72. This is a different form of characterization than the type performed in step 306. In step 306, individual ratios were characterized. In step 308 whole sets are characterized. In some embodiments, a set 72 is characterized as “positive” when more of the ratios defined by the set 72 are “positive” than are “negative”. The individual assignment of ratios in a set 72 as “positive” or negative” is made in step 306. To illustrate, consider the case in which a particular set 72 defines five ratios. Three of these ratios are determined to be “positive” and two of these ratios are determined to be negative in step 306. In this case, the set 72 is “positive” since it includes more positive ratios then negative ratios. The ratios sets 72 of each candidate sample class 56 are characterized in step 308 as described above. If only one of the ratios sets is characterized as positive, then the unclassified biological specimen is classified into the biological class 56 that corresponds to the lone positive set 72. A set 72 is characterized as “negative” when it includes more negative ratios than positive ratios. A set 72 is characterized as “indeterminate” when the number of positive ratios equals the number of negative ratios.

In many instances, the steps illustrated in FIG. 3 are used to validate the classifiers (the ratios sets 72) that were calculated in Section 5.1. To do this, a number of biological specimens of known biological classification are independently processed through steps 302, 304 and 306. Then, in step 308, each biological specimen S is classified as follows:
“true positive” when (i) the set corresponding to the true biological sample class (the sample class that the biological specimen actually belongs) of specimen S tests positive and (ii) the sets 72 of all other biological sample classes test negative or are indeterminate;
“false positive” when (i) a set 72 corresponding to a biological sample class 56 that originate from the same tissue (origin) as the true sample class of the specimen S tests positive and (ii) all other sets tested for specimen S test negative or indeterminate;
“false negative” when (i) a set 72 corresponding to a biological sample class 56 that does not originate from the same tissue type as the true biological sample class 56 of the biological specimen S tests positive and (ii) all other sets for specimen S test negative or indeterminate; and
“indeterminate” when none of the other conditions apply. The condition “false positive” can arise, for example, in the case where the problem to be addressed is the classification of a tumor of unknown primary origin. In such a case, and as described in the Experimental Section 6.0 below, one of the biological sample classes 72 is lung adenocarcinoma and another of the biological sample classes is lung squamous cell carcinoma. If step 308 incorrectly identifies a lung adenocarcinoma as lung squamous cell carcinoma, the lung adenocarcinoma biological specimen is labeled a “false positive”.

It will be appreciated that the bifurcation of incorrectly identified biological specimens into “false positives” and “false negatives” is purely a bookkeeping technique designed to provide more detail on such incorrect identifications and, as such, is entirely optional. Central to the techniques in accordance with this embodiment of the present invention is a “best of N” scheme in which N is the number of ratios in a given set 72. In other words, a set is considered “positive” (or true positive) when it includes more positive ratios then negative ratios (where positive ratios and negative ratios are as defined in step 306) and is negative (e.g., false positive or false negative) or indeterminate otherwise. However, in some embodiments of the present invention, a weighting scheme can be used where each true positive ratio in a set 72 is given a different weight than each true negative in the set 72. For example, each true positive ratio in a set 72 can be given a weight of 3.0 and each true negative ratio in the set can be given a weight of 1.0. In this weighting scheme, a set 72 will be considered positive even when the set 72 consists of one positive ratio and two negative ratios.

Step 308 concludes the characterization of an unclassified biological specimen into a biological sample class. It will be appreciated that a plurality of biological sample classes 56 are not needed to practice the methods described in FIG. 3. For example, there can be a single biological sample class 56 and, correspondingly, a single set of ratios 72. In such instances, the question becomes a consideration as to whether the unclassified biological specimen belongs to the single class 56 or not. For more information on how a set 72 (model) can be classified, see copending United States patent application U.S. Ser. No. ______ to be determined entitled “Knowledge-based Storage of Diagnostic Models” to Tran et al., attorney docket number 11373-004-888, that was filed on Sep. 29, 2003.

### 5.3. Exemplary Biological Sample Classes

The present invention can be used to develop models (sets of cellular constituent pairs) that distinguish between biological sample classes 56. A broad array of biological sample classes 56 are contemplated. In one example, two respective biological sample classes are (i) a wild type state and (ii) a diseased state. In another example two respective biological sample classes are (i) a first diseased state and a second diseased state. In still example two respective biological sample classes are (i) a drug respondent state versus a drug non-respondent state. In such instances, a first set 72 is developed for the first biological sample class and a second set 72 is developed for the second biological sample class. The present invention is not limited to instances where there are only two biological sample classes. Indeed there can be any number of biological sample classes (e.g., one biological sample class, two or more biological sample classes, between three and ten biological sample classes, between five and twenty biological sample classes, more than twenty-five biological sample classes, etc.). In such instances, a different set 72 is developed for each of the biological sample classes using the methods described in Section 5.1, above. This section describes exemplary references that can be used to develop biological sample classes. In addition, Section 5.3.9 discloses additional exemplary biological sample classes within the scope of the present invention.

### 5.3.1 Breast Cancer

Pustzai et al. Several different adjuvant chemotherapy regimens are used in the treatment of breast cancer. Not all regimens may be equally effective for all patients. Currently it is not possible to select the most effective regimen for a particular individual. One accepted surrogate of prolonged recurrence-free survival after chemotherapy in breast cancer is complete pathologic response (pCR) to neoadjuvant therapy. Pustzai et al., ASCO 2003 abstr 1 report the discovery of a gene expression profile that predicts pCR after neoadjuvant weekly paclitaxel followed by FAC sequential chemotherapy (T/FAC). The Pustzai et al. predictive markers were generated from fine needle aspirates of 24 early stage breast cancers. Six of the 24 patients achieved pCR (25 percent). In Pustzai et al., RNA from each sample were profiled on cDNA microarrays of 30,000 human transcripts. Differentially expressed genes between the pCR and residual disease (RD) groups were selected by signal-to-noise-ratio. Several supervised learning methods were evaluated to define the best class prediction algorithm and the optimal number of genes needed for outcome prediction using leave-one out cross validation. Support vector machine using five genes (3 ESTs, nuclear factor 1/A, and histone acetyltransferase) yielded the greatest estimated accuracy. This predictive marker set was tested on independent cases receiving T/FAC neoadjuvant therapy. Pustzai et al. reported results for 21 patients included in the validation. The overall accuracy of the Pustzai et al. response prediction based on gene expression profile was 81 percent. The overall specificity was 93 percent. The sensitivity was 50 percent (three of the six pCR were misclassified as RD). Pustzai et al. found that patients predicted to have pCR to T/FAC preoperative chemotherapy had a 75 percent chance of experiencing pCR compared to 25-30 percent that is expected in unselected patients. The Pustzai et al. findings can be used as source models in the methods described in Section 5.1, above, in order to develop a classifier that can then be used to help physicians to select individual patients who are most likely to benefit from T/FAC adjuvant chemotherapy.

Cobleigh et al. Breast cancer patients with ten or more positive nodes have a poor prognosis, yet some survive long-term. Cobleigh et al., ASCO 2003 abstr 3415 sought to identify predictors of distant disease-free survival (DDFS) in this high risk group of patients. Patients with invasive breast cancer and ten or more positive nodes diagnosed from 1979 to 1999 were identified. RNA was extracted from three 10 micron sections and expression was quantified for seven reference genes and 185 cancer-related genes using RT-PCR. The genes were selected based on the results of published literature and microarray experiments. A total of 79 patients were studied. Fifty-four percent of the patients received hormonal therapy and eighty percent received chemotherapy. Median follow-up was 15.1 yrs. As of August 2002, 77 percent of patients had distant recurrence or breast cancer death. Univariate Cox survival analysis of the clinical variables indicated that number of nodes involved was significantly associated with DDFS (p=0.02). Cobleigh et al. applied a multivariate model including age, tumor size, involved nodes, tumor grade, adjuvant hormonal therapy, and chemotherapy that accounted for 13 percent of the variance in DDFS time. Univariate Cox survival analysis of the 185 cancer-related genes indicated that a number of genes were associated with DDFS (5 with p<0.01; 16 with p<0.05). Higher expression was associated with shorter DDFS (p<0.01) for the HER2 adaptor Grb7 and the macrophage marker CD68. Higher expression was associated with longer DDFS (p<0.01) for TP53BP2 (tumor protein p53-binding protein 2), PR, and Bcl2. A multivariate model including five genes accounted for 45 percent of the variance in DDFS time. Multivariate analysis also indicated that gene expression is a significant predictor after controlling for clinical variables. The Cobleigh et al. findings can be used as source models in the methods described in Section 5.1, above, to develop a classifier that can then be used to help determine which patients are likely associated with DDFS and that are not likely associated with DDFS.

van't Veer. Breast cancer patients with the same stage of disease can have markedly different treatment responses and overall outcome. Predictors for metastasis (a poor outcome), lymph node status and histological grade, for example fail to classify accurately breast tumors according to their clinical behavior. To address this shortcoming vant't Veer 2002, Nature 415, 530-535, used DNA microanalysis on primary breast tumors of 117 patients, and applied supervised classification to identify a gene expression signature strongly predictive of a short interval to distant metastases (‘poor prognosis’ signature) in patients without tumor cells in local lymph nodes at diagnosis (lymph node negative). In addition vant't Veer established a signature that identifies tumors of BRCA1 carriers. The van't Veer findings can be used as source models in the methods described in Section 5.1, above, to develop a classifier that determines breast cancer patient prognosis.

Other references. A representative sample of additional breast cancer studies that can be used as source models to develop classifiers for breast cancer include, but are not limited to, Soule et al., ASCO 2003 abstr 3466; Ikeda et al., ASCO 2003 abstr 34; Schneider et al., 2003, British Journal of Cancer 88, p. 96; Long et al. ASCO 2003 abstr 3410; and Chang et al., 2002, PeerView Press, Abstract 1700, “Gene Expression Profiles for Docetaxel Chemosensitivity”.

### 5.3.2 Lung Cancer

Rosell-Costa et al. ERCC 1 mRNA levels correlate with DNA repair capacity (DRC) and clinical resistance to cisplatin. Changes in enzyme activity and gene expression of the M1 or M2 subunits of ribonucleotide reductase (RR) are observed during DNA repair after gemcitabine damage. Rosell-Costa et al., ASCO 2003 abstr 2590 assessed ERCC1 and RRM1 mRNA levels by quantitative PCR in RNA isolated from tumor biopsies of 100 stage 1V (NSCLC) patients included in a trial of 570 patients randomized to gem/cis versus gem/cis/vrb vs gem/vrb followed by vrb/ifos (Alberola et al. ASCO 2001 abstr 1229). ERCC1 and RRM1 data was available for 81 patients. Overall response rate, time to progression (TTP) and median survival (MS) for these 81 patients were similar to results for all 570 patients. A strong correlation between ERCC1 and RRM1 levels was found (P=0.00001). Significant differences in outcome according to ERCC1 and RRM1 levels was found in the gem/cis arm but not in the other arms. In the gem/cis arm, TTP was 8.3 months for patients with low ERCC 1 and 5.1 months for patients with high ERCC 1 (P=0.07), 8.3 months for patients with low RRM1 and 2.7 months for patients with high RRM1 (P=0.01), 10 months for patients with low ERCC1 & RRM1 and 4.1 months for patients with high ERCC1 & RRM1 (P=0.009). MS was 13.7 months for patients with low ERCC1 and 9.5 months for patients with high ERCC1 (P=0.19), 13.7 months for patients with low RRM1 and 3.6 months for patients with high RRM1 (P=0.009), not reached for patients with low ERCC1 & RRM1 and 6.8 months for patients with high ERCC1 & RRM1 (P=0.004). Patients with low ERCC1 and RRM1 levels, indicating low DRC, are ideal candidates for gem/cis, while patients with high levels have poorer outcome. Accordingly, ratios that include ERCC1 & RRM1 can be used as source models in the methods outlined in Section 5.1 in order to determine what kind of therapy should be given to lung cancer patients.

Hayes et al. Despite the high prevalence of lung cancer, a robust stratification of patients by prognosis and treatment response remains elusive. Initial studies of lung cancer gene expression arrays have suggested that previously unrecognized subclasses of adenocarcinoma may exist. These studies have not been replicated and the association of subclass with clinical outcomes remains incomplete. For the purpose of comparing subclasses suggested by the three largest case series, their gene expression arrays comprising 366 tumors and normal tissue samples were analyzed in a pooled data set by Hayes et al., ASCO 2003 abstr 2526. The common set of expression data was re-scaled and gene filtering was employed to select a subset of genes with consistent expression between replicate pairs yet variable expression across all samples. Hierarchical clustering was performed on the common data set and the resultant clusters compared to those proposed by the authors of the original manuscripts. In order to make direct comparisons to the original classification schemes, a classifier was constructed and applied to validation samples from the pool of 366 tumors. In each step of the analysis, the clustering agreement between the validation and the originally published classes was good and strongly statistically significant. In an additional validation step, the lists of genes describing the originally published subclasses where compared across classification schemes. Again there was statistically significant overlap in the lists of genes used to describe adenocarcinoma subtypes. Finally, survival curves demonstrated one subtype of adenocarcinoma with consistently decreased survival. The Hayes et al. analyses helps to establish that reproducible adenocarcinoma subtypes can be described based on mRNA expression profiling. Accordingly the results of Hayes et al. can be used as a source model in the methods described in Section 5.1, above. Classifiers (sets 72) developed in this way can then be used to identify adenocarcinoma subtypes using the techniques outlined in Section 5.2, above.

### 5.3.3 Prostate Cancer

Li et al. Taxotere shows anti-tumor activity against solid tumors including prostate cancer. However, the molecular mechanism(s) of action of Taxotere have not been fully elucidated. In order to establish the molecular mechanism of action of Taxotere in both hormone insensitive (PC3) and sensitive (LNCaP) prostate cancer cells comprehensive gene expression profiles were obtained by using Affymetrix Human Genome U133A array. See Li et al. ASCO 2003 abstr 1677. The total RNA from cells untreated and treated with 2 nM Taxotere for 6, 36, and 72 hours was subjected to microarray analysis and the data were analyzed using Microarray Suite and Data Mining, Cluster and TreeView, and Onto-express software. The alternations in the expression of genes were observed as early as six hours, and more genes were altered with longer treatments. Additionally, Taxotere exhibited differential effects on gene expression profiles between LNCaP and PC3 cells. A total of 166, 365, and 1785 genes showed >2 fold change in PC3 cells after 6, 36, and 72 hours, respectively compared to 57, 823, and 964 genes in LNCaP cells. Li et al. found no effect on androgen receptor, although up-regulation of several genes involved in steroid-independent AR activation (IGFBP2, FGF13, EGF8, etc) was observed in LNCaP cells. Clustering analysis showed down-regulation of genes for cell proliferation and cell cycle (cyclins and CDKs, Ki-67, etc), signal transduction (IMPA2, ERBB21P, etc), transcription factors (HMG-2, NFYB, TRIP13, PIR, etc), and oncogenesis (STK15, CHK1, Survivin, etc) in both cell lines. In contrast, Taxotere up-regulated genes that are related to induction of apoptosis (GADD45A, FasApo-1, etc), cell cycle arrest (p21CIP 1, p27KIP1, etc) and tumor suppression. From these results, Li et al. concluded that Taxotere caused alterations of a large number of genes, many of which may contribute to the molecular mechanism(s) by which Taxotere affects prostate cancer cells. This information could be further exploited to devise strategies to optimize therapeutic effects of Taxotere for the treatment of metastatic prostate cancer.

The methods described in Section 5.1 can be used to develop classifiers that stratify patients into groups that will have a varying degree of response to Taxotere and related treatment regimens (e.g. a first biological sample class that is highly responsive to Taxotere, a second biological sample class that is not responsive to Taxotere, etc.). In another approach, biological sample classes can be developed based, in part, on Cox-2 expression in order to serve as a survival predictor in stage D2 prostate cancer.

### 5.3.4 Colorectal Cancer

Kwon et al. To identify a set of genes involved in the development of colorectal carcinogenesis, Kwon et al. ASCO 2003 abstr 1104 analyzed gene-expression profiles of colorectal cancer cells from twelve tumors with corresponding noncancerous colonic epithelia by means of a cDNA microarray representing 4,608 genes. Kwon et al. classified both samples and genes by a two-way clustering analysis and identified genes that were differentially expressed between cancer and noncancerous tissues. Alterations in gene expression levels were confirmed by reverse-transcriptase PCR (RT-PCR) in selected genes. Gene expression profiles according to lymph node metastasis were evaluated with a supervised learning technique. Expression change in more than 75 percent of the tumors was observed for 122 genes, i.e., 77 up-regulated and 45 down-regulated genes. The most frequently altered genes belonged to functional categories of signal transduction (19 percent), metabolism (17 percent), cell structure/motility (14 percent), cell cycle (13 percent) and gene protein expression (13 percent). The RT-PCR analysis of randomly selected genes showed consistent findings with those in cDNA microarray. Kwon et al. could predict lymph node metastasis for 10 out of 12 patients with cross-validation loops. The results of Kwon et al. can be used as a source model in the methods outlined in Section 5.1, above, in order to build a classifier for determining whether a patient has colorectal cancer. Furthermore, the classifiers could be extended to identify subclasses of colorectal cancer.

Additional studies that can be used as source models to develop classifiers for colorectal cancer (including classifiers that identify a biological specimen as having colorectal cancer and possibly additional classifiers that predict subgroups of colorectal cancer) include, but are not limited to Nasir et al., 2002, In Vivo. 16, p. 501 in which research that finds elevated expression of COX-2 has been associated with tumor induction and progression is summarized, as well as Longley et al., 2003 Clin. Colorectal Cancer. 2, p. 223; McDermott et al., 2002, Ann Oncol. 13, p. 235; and Longley et al., 2002, Pharmacogenomics J. 2, p. 209.

### 5.3.5 Ovarian Cancer

Spentzos et al. To identify expression profiles associated with clinical outcomes in epithelial ovarian cancer (EOC), Spentzos et al. ASCO 2003 abstr 1800 evaluated 38 tumor samples from patients with EOC receiving first-line platinum/taxane-based chemotherapy. RNA probes were reverse-transcribed, fluorescent-labeled, and hybridized to oligonucleotide arrays containing 12675 human genes and expressed sequence tags. Expression data were analyzed for signatures predictive of chemosensitivity, disease-free survival (DFS) and overall survival (OS). A Bayesian model was used to sort the genes according to their probability of differential expression between tumors of different chemosensitivity and survival. Genes with the highest probability of being differentially expressed between tumor subgroups with different outcome were included in the respective signature. Spentzos et al. found one set of genes that were overexpressed in chemoresistant tumors and another set of genes that were overexpressed in chemosensitive tumors. Spentzos et al. found 45 genes that were overexpressed in tumors associated with short disease free survival (DFS) and 18 genes that were overexpressed in tumors associated with long DFS. These genes separated the patient population into two groups with median DFS of 7.5 and 30.5 months (p<0.00001). Spentzos et al. found 20 genes that were overexpressed in tumors with short overall survival (OS) and 29 genes that were overexpressed in genes with long OS (median OS of 22 and 40 months, p=0.00008). The overexpressed genes identified by Spentzos et al. can serve as a source model (see FIG. 2A) for the methods of Section 5.1 in order to build classifiers that can classify a biological specimen into biological classes such as chemoresistant ovarian cancer, chemosensitive ovarian cancer, short DFS ovarian cancer, long DFS ovarian cancer, short OS ovarian cancer and long OS ovarian cancer.

Additional studies that can be used as source models for ovarian cancer include, but are not limited to, Presneau et al., 2003, Oncogene 13, p. 1568; and Takano et al. ASCO 2003 abstr 1856.

### 5.3.6 Bladder Cancer

Wulfing et al. Cox-2, an inducible enzyme involved in arachidonate metabolism, has been shown to be commonly overexpressed in various human cancers. Recent studies have revealed that Cox-2 expression has prognostic value in patients who undergo radiation or chemotherapy for certain tumor entities. In bladder cancer, Cox-2 expression has not been well correlated with survival data is inconsistent. To address this, Wulfing et al. ASCO 2003 abstr 1621 studied 157 consecutive patients who had all undergone radical cystectomy for invasive bladder cancer. Of these, 61 patients had received cisplatin-containing chemotherapy, either in an adjuvant setting or for metastatic disease. Standard immunohistochemistry was performed on paraffin-embedded tissue blocks applying a monoclonal Cox-2 antibody. Semiquantitative results were correlated to clinical and pathological data, long-term survival rates (3-177 months) and details on chemotherapy. 26 (16.6 percent) cases were Cox-2-negative. From all positive cases (n=131, 83.4 percent), 59 (37.6 percent) showed low, 53 (33.8 percent) moderate and 19 (12.1 percent) strong Cox-2 expression. Expression was independent of TNM-Staging and histological grading. Cox-2 expression correlated significantly with the histological type of the tumors (urothelial vs. squamous cell carcinoma; P=0.01). In all investigated cases, Kaplan-Meier analysis did not show any statistical correlation to overall and disease free survival. However, by subgroup analysis of those patients having received cisplatin-containing chemotherapy, Cox-2-expression was significantly related to poor overall survival time (P=0.03). According to Wulfing et al., immunohistochemical overexpression of Cox-2 is a very common event in bladder cancer. Patients receiving chemotherapy seem to have worse survival rates when overexpressing Cox-2 in their tumors. Therefore, Wulfing et al. reasoned that Cox-2 expression could provide additional prognostic information for patients with bladder cancer treated with cisplatin-based chemotherapy regimens and that this could be the basis for a more aggressive therapy in individual patients or a risk-adapted targeted therapy using selective Cox-2-inhibitors. The results of Wulfing et al. could be used as a source model (possibly along with other marker genes) for the development of sets 72 that stratify a bladder cancer population into treatment groups using the methods outlined in Sections 5.1 and 5.2 above.

### 5.3.7 Gastric Cancer

Terashima et al. In order to detect the chemoresistance-related gene in human gastric cancer, Terashima et al., ASCO 2003 abstr 1161 investigated gene expression profiles using DNA microarray and compared the results with in vitro drug sensitivity. Fresh tumor tissue was obtained from a total of sixteen patients with gastric cancer and then examined for gene expression profile using GeneChip Human U95Av2 array (Affymetrix, Santa Clara, Calif.), which includes 12,000 human genes and EST sequences. The findings were compared with the results of in vitro drug sensitivity determined by a ATP assay. The investigated drugs and drug concentrations were cisplatin (CDDP), doxorubicin (DOX), mitomycin C (MMC), etoposide (ETP), irinotecan (CPT; as SN-38), 5-fluoruuracil (5-FU), doxifluridine (5′-DFUR), paclitaxel (TXL) and docetaxel (TXT). Drug was added at a concentration of Cₘₐₓ of each drug for 72 hours. Drug sensitivity was expressed as the ratio of the ATP content in drug treated group to control group (T/C percent). Pearson correlation between the amount of relative gene expression and T/C percent was evaluated and clustering analysis was also performed y using genes selected by the correlation. From these analyses, 51 genes in CDDP, 34 genes in DOX, 26 genes in MMC, 52 genes in ETP, 51 genes in CPT, 85 genes in 5-FU, 42 genes in 5′-DFUR, 11 genes in TXL and 32 genes in TXT were up-regulated in drug resistant tumors. Most of these genes were related to cell growth, cell cycle regulation, apoptosis, heat shock protein or ubiquitin-proteasome pathways. However, several genes were specifically up-regulated in each drug-resistant tumors, such as ribosomal proteins, CD44 and elongation factor alpha 1 in CDDP. The up-regulated genes identified by Terashima et al. can be used as source models in the methods described in Section 5.1 in order to develop ratios sets 72 that not only diagnose patients with gastric cancer, but provide an indication of whether the patient has a drug-resistant gastric tumor and, if so, which kind of drug-resistant tumor.

Additional references that can be used as a source models for gastric cancer include, but are not limited to Kim et al. ASCO 2003 abstr 560; Arch-Ferrer et al. ASCO 2003 abstr 1101; Hobday ASCO 2003 abstr 1078; Song et al. ASCO 2003 abstr 1056 (overexpression of the Rb gene is an independent prognostic factor for predicting relapse free survival); Leichman et al., ASCO 2003 abstr 1054 (thymidylate synthase expression as a predictor of chemobenefit in esophageal/gastric cancer).

### 5.3.8 Rectal Cancer

Lenz et al. Local recurrence is a significant clinical problem in patients with rectal cancer. Accordingly, Lenz et al. ASCO 2003 abstr 1185 sought to establish a genetic profile that would predict pelvic recurrence in patients with rectal cancer treated with adjuvant chemoradiation. A total of 73 patients with locally advanced rectal cancer (UICC stage II and III), 25 female, 48 male, median age 52.1 years, were treated from 1991-2000. Histological staging categorized 22 patients as stage T2, 51 as stage T3. A total of 35 patients were lymph node negative, 38 had one or more lymph node metastases. All patients underwent cancer resection, followed by 5-FU plus pelvic radiation. RNA was extracted from formalin-fixed, paraffin-embedded, laser-capture-microdissected tissue. Lenz et al. determined mRNA levels of genes involved in the 5FU pathway (TS, DPD), angiogenesis (VEGF), and DNA repair (ERCC1, RAD51) in tumor and adjacent normal tissue by quantitative RT-PCR (Taqman). Lenz et al. found a significant association between local tumor recurrence and higher m-RNA expression levels in adjacent normal tissue of ERCC1 and TS suggest that gene expression levels of target genes of the 5-FU pathways as well as DNA repair and angiogenesis may be useful to identify patients at risk for pelvic recurrence. The results of Lenz et al. can be used as a source model for developing a set of ratios 72 that, when used in accordance with the methods described in Section 5.2, above, identify patients at risk for pelvic recurrence.

### 5.3.9 Additional Exemplary Biological Sample Classes

Additional representative biological sample classes include, but are not limited to, acne, acromegaly, acute cholecystitis, Addison's disease, adenomyosis, adult growth hormone deficiency, adult soft tissue sarcoma, alcohol dependence, allergic rhinitis, allergies, alopecia, alzheimer disease, amniocentesis, anemia in heart failure, anemias, angina pectoris, ankylosing spondylitis, anxiety disorders, arrhenoblastoma of ovary, arrhythmia, arthritis, arthritis-related eye problems, asthma, atherosclerosis, atopic eczema atrophic vaginitis, attention deficit disorder, attention disorder, autoimmune diseases, balanoposthitis, baldness, bartholins abscess, birth defects, bleeding disorders, bone cancer, brain and spinal cord tumors, brain stem glioma, brain tumor, breast cancer, breast cancer risk, breast disorders, cancer, cancer of the kidney, cardiomyopathy, carotid artery disease, carotid endarterectomy, carpal tunnel syndrome, cerebral palsy, cervical cancer, chancroid, chickenpox, childhood nephrotic syndrome, chlamydia, chronic diarrhea, chronic heart failure, claudication, colic, colon or rectum cancer, colorectal cancer, common cold, condyloma (genital warts), congenital goiters, congestive heart failure, conjunctivitis, corneal disease, comeal ulcer, coronary heart disease, cryptosporidiosis, Cushings syndrome, cystic fibrosis, cystitis, cystoscopy or ureteroscopy, De Quervains disease, dementia, depression, mania, diabetes, diabetes insipidus, diabetes mellitus, diabetic retinopathy, Down syndrome, dysmenorrhea in the adolescent, dyspareunia, ear allergy, ear infection, eating disorder, eczema, emphysema, endocarditis, endometrial cancer, endometriosis, eneuresis in children, epididymitis, epilepsy, episiotomy, erectile dysfunction, eye cancer, fatal abstraction, fecal incontinence, female sexual dysfunction, fetal abnormalities, fetal alcohol syndrome, fibromyalgia, flu, folliculitis, fungal infection, gardnerella vaginalis, genital candidiasis, genital herpes, gestational diabetes, glaucoma, glomerular diseases, gonorrhea, gout and pseudogout, growth disorders, gum disease, hair disorders, halitosis, Hamburger disease, hemophilia, hepatitis, hepatitis b, hereditary colon cancer, herpes infection, human placental lactogen, hyperparathyroidism, hypertension, hyperthyroidism, hypoglycemia, hypogonadism, hypospadias, hypothyroidism, hysterectomy, impotence, infertility, inflammatory bowel disease, inguinal hernia, inherited heart irregularity, intraocular melanoma, irritable bowel syndrome, Kaposis sarcoma, leukemia, liver cancer, lung cancer, lung disease, malaria, manic depressive illness, measles, memory loss, meningitis in children, menorrhagia, mesothelioma, microalbumin, migraine headache, mittelschmerz, mouth cancer, movement disorders, mumps, Nabothian cyst, narcolepsy, nasal allergies, nasal cavity and paranasal sinus cancer, neuroblastoma, neurofibromatosis, neurological disorders, newborn jaundice, obesity, obsessive-compulsive disorder, orchitis or epididymitis, orofacial myofunctional disorders, osteoarthritis, osteoporosis, osteoporosis, osteosarcoma, ovarian cancer, ovarian cysts, pancreatic cancer, paraphimosis, Parkinson disease, partial epilepsy, pelvic inflammatory disease, peptic ulcer, peripartum cardiomyopathy, peyronie disease, polycystic ovary syndrome, preeclampsia, pregnanediol, premenstrual syndrome, priapism, prolactinoma, prostate cancer, psoriasis, rheumatic fever, salivary gland cancer, SARS, sexually transmitted diseases, sexually transmitted enteric infections, sexually transmitted infections, Sheehans syndrome, sinusitis, skin cancer, sleep disorders, smallpox, smell disorders, snoring, social phobia, spina bifida, stomach cancer, syphilis, testicular cancer, thyroid cancer, thyroid disease, tonsillitis, tooth disorders, trichomoniasis, tuberculosis, tumors, type II diabetes, ulcerative colitis, urinary tract infections, urological cancers, uterine fibroids, vaginal cancer, vaginal cysts, vulvodynia, and vulvovaginitis.

### 5.4 Transcriptional State Measurements

This section provides some exemplary methods for measuring the expression level of genes, which are one type of cellular constituent. One of skill in the art will appreciate that this invention is not limited to the following specific methods for measuring the expression level of genes in each organism in a plurality of organisms.

### 5.4.1 Transcript Assay using Microarrays

The techniques described in this section include the provision of polynucleotide probe arrays that can be used to provide simultaneous determination of the expression levels of a plurality of genes. These technique further provide methods for designing and making such polynucleotide probe arrays.

The expression level of a nucleotide sequence in a gene can be measured by any high throughput techniques. However measured, the result is either the absolute or relative amounts of transcripts or response data, including but not limited to values representing characteristics or characteristic ratios. Preferably, measurement of the expression profile is made by hybridization to transcript arrays, which are described in this subsection. In one embodiment, “transcript arrays” or “profiling arrays” are used. Transcript arrays can be employed for analyzing the expression profile in a cell sample and especially for measuring the expression profile of a cell sample of a particular tissue type or developmental state or exposed to a drug of interest.

In one embodiment, an expression profile is obtained by hybridizing detectably labeled polynucleotides representing the nucleotide sequences in mRNA transcripts present in a cell (e.g., fluorescently labeled cDNA synthesized from total cell mRNA) to a microarray. A microarray is an array of positionally-addressable binding (e.g., hybridization) sites on a support for representing many of the nucleotide sequences in the genome of a cell or organism, preferably most or almost all of the genes. Each of such binding sites consists of polynucleotide probes bound to the predetermined region on the support. Microarrays can be made in a number of ways, of which several are described herein below. However produced, microarrays share certain characteristics. The arrays are reproducible, allowing multiple copies of a given array to be produced and easily compared with each other. Preferably, the microarrays are made from materials that are stable under binding (e.g., nucleic acid hybridization) conditions. Microarrays are preferably small, e.g., between 1 cm² and 25 cm², preferably 1 to 3 cm². However, both larger and smaller arrays are also contemplated and may be preferable, e.g., for simultaneously evaluating a very large number or very small number of different probes.

Preferably, a given binding site or unique set of binding sites in the microarray will specifically bind (e.g., hybridize) to a nucleotide sequence in a single gene from a cell or organism (e.g., to exon of a specific mRNA or a specific cDNA derived therefrom).

The microarrays used can include one or more test probes, each of which has a polynucleotide sequence that is complementary to a subsequence of RNA or DNA to be detected. Each probe typically has a different nucleic acid sequence, and the position of each probe on the solid surface of the array is usually known. Indeed, the microarrays are preferably addressable arrays, more preferably positionally addressable arrays. Each probe of the array is preferably located at a known, predetermined position on the solid support so that the identity (i.e., the sequence) of each probe can be determined from its position on the array (i.e., on the support or surface). In some embodiments, the arrays are ordered arrays.

Preferably, the density of probes on a microarray or a set of microarrays is 100 different (i.e., non-identical) probes per 1 cm² or higher. More preferably, a microarray used in the methods of the invention will have at least 550 probes per 1 cm², at least 2,000 probes per 1 cm², at least 4,000 probes per 1 cm² or at least 10,000 probes per 1 cm². In a particularly preferred embodiment, the microarray is a high density array, preferably having a density of at least 15,000 different probes per 1 cm². The microarrays used in the invention therefore preferably contain at least 25,000, at least 50,000, at least 100,000, at least 150,000, at least 200,000, at least 250,000, at least 500,000 or at least 550,000 different (e.g., non-identical) probes.

In one embodiment, the microarray is an array (e.g., a matrix) in which each position represents a discrete binding site for a nucleotide sequence of a transcript encoded by a gene (e.g., for an exon of an mRNA or a cDNA derived therefrom). The collection of binding sites on a microarray contains sets of binding sites for a plurality of genes. For example, in various embodiments, the microarrays of the invention can comprise binding sites for products encoded by fewer than 50 percent of the genes in the genome of an organism. Alternatively, the microarrays of the invention can have binding sites for the products encoded by at least 50 percent, at least 75 percent, at least 85 percent, at least 90 percent, at least 95 percent, at least 99 percent or 100 percent of the genes in the genome of an organism. In other embodiments, the microarrays of the invention can having binding sites for products encoded by fewer than 50 percent, by at least 50 percent, by at least 75 percent, by at least 85 percent, by at least 90 percent, by at least 95 percent, by at least 99 percent or by 100 percent of the genes expressed by a cell of an organism. The binding site can be a DNA or DNA analog to which a particular RNA can specifically hybridize. The DNA or DNA analog can be, e.g., a synthetic oligomer or a gene fragment, e.g. corresponding to an exon.

In some embodiments of the present invention, a gene or an exon in a gene is represented in the profiling arrays by a set of binding sites comprising probes with different polynucleotides that are complementary to different sequence segments of the gene or the exon. Such polynucleotides are preferably of the length of 15 to 200 bases, more preferably of the length of 20 to 100 bases, most preferably 40-60 bases. Each probe sequence may also comprise linker sequences in addition to the sequence that is complementary to its target sequence. As used herein, a linker sequence is a sequence between the sequence that is complementary to its target sequence and the surface of support. For example, in preferred embodiments, the profiling arrays of the invention comprise one probe specific to each target gene or exon. However, if desired, the profiling arrays may contain at least 2, 5, 10, 100, or 1000 or more probes specific to some target genes or exons. For example, the array may contain probes tiled across the sequence of the longest mRNA isoform of a gene at single base steps.

In specific embodiments of the invention, when an exon has alternative spliced variants, a set of polynucleotide probes of successive overlapping sequences, i.e., tiled sequences, across the genomic region containing the longest variant of an exon can be included in the exon profiling arrays. The set of polynucleotide probes can comprise successive overlapping sequences at steps of a predetermined base intervals, e.g. at steps of 1, 5, or 10 base intervals, span, or are tiled across, the mRNA containing the longest variant. Such sets of probes therefore can be used to scan the genomic region containing all variants of an exon to determine the expressed variant or variants of the exon to determine the expressed variant or variants of the exon. Alternatively or additionally, a set of polynucleotide probes comprising exon specific probes and/or variant junction probes can be included in the exon profiling array. As used herein, a variant junction probe refers to a probe specific to the junction region of the particular exon variant and the neighboring exon. In some cases, the probe set contains variant junction probes specifically hybridizable to each of all different splice junction sequences of the exon. In other cases, the probe set contains exon specific probes specifically hybridizable to the common sequences in all different variants of the exon, and/or variant junction probes specifically hybridizable to the different splice junction sequences of the exon.

In some cases, an exon is represented in the exon profiling arrays by a probe comprising a polynucleotide that is complementary to the full length exon. In such instances, an exon is represented by a single binding site on the profiling arrays. In some preferred cases, an exon is represented by one or more binding sites on the profiling arrays, each of the binding sites comprising a probe with a polynucleotide sequence that is complementary to an RNA fragment that is a substantial portion of the target exon. The lengths of such probes are normally between 15-600 bases, preferably between 20-200 bases, more preferably between 30-100 bases, and most preferably between 40-80 bases. The average length of an exon is about 200 bases (see, e.g., Lewin, Genes V, Oxford University Press, Oxford, 1994). A probe of length of 40-80 allows more specific binding of the exon than a probe of shorter length, thereby increasing the specificity of the probe to the target exon. For certain genes, one or more targeted exons may have sequence lengths less than 40-80 bases. In such cases, if probes with sequences longer than the target exons are to be used, it may be desirable to design probes comprising sequences that include the entire target exon flanked by sequences from the adjacent constitutively splice exon or exons such that the probe sequences are complementary to the corresponding sequence segments in the mRNAs. Using flanking sequence from adjacent constitutively spliced exon or exons rather than the genomic flanking sequences, i.e., intron sequences, permits comparable hybridization stringency with other probes of the same length. Preferably the flanking sequence used are from the adjacent constitutively spliced exon or exons that are not involved in any alternative pathways. More preferably the flanking sequences used do not comprise a significant portion of the sequence of the adjacent exon or exons so that cross-hybridization can be minimized. In some embodiments, when a target exon that is shorter than the desired probe length is involved in alternative splicing, probes comprising flanking sequences in different alternatively spliced mRNAs are designed so that expression level of the exon expressed in different alternatively spliced mRNAs can be measured.

In some instances, when alternative splicing pathways and/or exon duplication in separate genes are to be distinguished, the DNA array or set of arrays can also comprise probes that are complementary to sequences spanning the junction regions of two adjacent exons. Preferably, such probes comprise sequences from the two exons which are not substantially overlapped with probes for each individual exons so that cross hybridization can be minimized. Probes that comprise sequences from more than one exons are useful in distinguishing alternative splicing pathways and/or expression of duplicated exons in separate genes if the exons occurs in one or more alternative spliced mRNAs and/or one or more separated genes that contain the duplicated exons but not in other alternatively spliced mRNAs and/or other genes that contain the duplicated exons. Alternatively, for duplicate exons in separate genes, if the exons from different genes show substantial difference in sequence homology, it is preferable to include probes that are different so that the exons from different genes can be distinguished.

It will be apparent to one skilled in the art that any of the probe schemes, supra, can be combined on the same profiling array and/or on different arrays within the same set of profiling arrays so that a more accurate determination of the expression profile for a plurality of genes can be accomplished. It will also be apparent to one skilled in the art that the different probe schemes can also be used for different levels of accuracies in profiling. For example, a profiling array or array set comprising a small set of probes for each exon may be used to determine the relevant genes and/or RNA splicing pathways under certain specific conditions. An array or array set comprising larger sets of probes for the exons that are of interest is then used to more accurately determine the exon expression profile under such specific conditions. Other DNA array strategies that allow more advantageous use of different probe schemes are also encompassed.

Preferably, the microarrays used in the invention have binding sites (i.e., probes) for sets of exons for one or more genes relevant to the action of a drug of interest or in a biological pathway of interest. As discussed above, a “gene” is identified as a portion of DNA that is transcribed by RNA polymerase, which may include a 5′ untranslated region (“UTR”), introns, exons and a 3′ UTR. The number of genes in a genome can be estimated from the number of mRNAs expressed by the cell or organism, or by extrapolation of a well characterized portion of the genome. When the genome of the organism of interest has been sequenced, the number of ORFs can be determined and mRNA coding regions identified by analysis of the DNA sequence. For example, the genome of Saccharomyces cerevisiae has been completely sequenced and is reported to have approximately 6275 ORFs encoding sequences longer than 99 amino acid residues in length. Analysis of these ORFs indicates that there are 5,885 ORFs that are likely to encode protein products (Goffeau et al., 1996, Science 274: 546-567). In contrast, the human genome is estimated to contain approximately 30,000 to 130,000 genes (see Crollius et al., 2000, Nature Genetics 25: 235-238; Ewing et al., 2000, Nature Genetics 25: 232-234). Genome sequences for other organisms, including but not limited to Drosophila, C. elegans, plants, e.g., rice and Arabidopsis, and mammals, e.g., mouse and human, are also completed or nearly completed. Thus, in preferred embodiments of the invention, an array set comprising in total probes for all known or predicted exons in the genome of an organism is provided. As a non-limiting example, the present invention provides an array set comprising one or two probes for each known or predicted exon in the human genome.

It will be appreciated that when cDNA complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (i.e., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In one embodiment, cDNAs from cell samples from two different conditions are hybridized to the binding sites of the microarray using a two-color protocol. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA derived from each of the two cell types are differently labeled (e.g., with Cy3 and Cy5) so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in characteristic of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, change the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Quantitative monitoring of gene expression patterns with a complementary DNA microarray, Science 270: 467-470, which is incorporated by reference in its entirety for all purposes. The scheme is equally applicable to labeling and detection of exons. An advantage of using cDNA labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell. Furthermore, labeling with more than two colors is also contemplated in the present invention. In some embodiments of the invention, at least 5, 10, 20, or 100 dyes of different colors can be used for labeling. Such labeling permits simultaneous hybridizing of the distinguishably labeled cDNA populations to the same array, and thus measuring, and optionally comparing the expression levels of, mRNA molecules derived from more than two samples. Dyes that can be used include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41, cyamine dyes, including but are not limited to Cy3, Cy3.5 and Cy5; BODIPY dyes including but are not limited to BODIPY-FL, BODIPY-TR, BODIPY-TMR, BODIPY-630/650, and BODIPY-650/670; and ALEXA dyes, including but are not limited to ALEXA-488, ALEXA-532, ALEXA-546, ALEXA-568, and ALEXA-594; as well as other fluorescent dyes which will be known to those who are skilled in the art.

In some embodiments of the invention, hybridization data are measured at a plurality of different hybridization times so that the evolution of hybridization levels to equilibrium can be determined. In such embodiments, hybridization levels are most preferably measured at hybridization times spanning the range from 0 to in excess of what is required for sampling of the bound polynucleotides (i.e., the probe or probes) by the labeled polynucleotides so that the mixture is close to or substantially reached equilibrium, and duplexes are at concentrations dependent on affinity and abundance rather than diffusion. However, the hybridization times are preferably short enough that irreversible binding interactions between the labeled polynucleotide and the probes and/or the surface do not occur, or are at least limited. For example, in embodiments wherein polynucleotide arrays are used to probe a complex mixture of fragmented polynucleotides, typical hybridization times may be approximately 0-72 hours. Appropriate hybridization times for other embodiments will depend on the particular polynucleotide sequences and probes used, and may be determined by those skilled in the art (see, e.g., Sambrook et al., Eds., 1989, Molecular Cloning: A Laboratory Manual, 2nd ed., Vol. 1-3, Cold Spring Harbor Laboratory, Cold Spring Harbor, N.Y.).

In one embodiment, hybridization levels at different hybridization times are measured separately on different, identical microarrays. For each such measurement, at hybridization time when hybridization level is measured, the microarray is washed briefly, preferably in room temperature in an aqueous solution of high to moderate salt concentration (e.g., 0.5 to 3 M salt concentration) under conditions which retain all bound or hybridized polynucleotides while removing all unbound polynucleotides. The detectable label on the remaining, hybridized polynucleotide molecules on each probe is then measured by a method which is appropriate to the particular labeling method used. The resulted hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, one can use one array, hybridize for a short time, wash and measure the hybridization level, put back to the same sample, hybridize for another period of time, wash and measure again to get the hybridization time curve.

Preferably, at least two hybridization levels at two different hybridization times are measured, a first one at a hybridization time that is close to the time scale of cross-hybridization equilibrium and a second one measured at a hybridization time that is longer than the first one. The time scale of cross-hybridization equilibrium depends, inter alia, on sample composition and probe sequence and may be determined by one skilled in the art. In preferred embodiments, the first hybridization level is measured at between 1 to 10 hours, whereas the second hybridization time is measured at 2, 4, 6, 10, 12, 16, 18, 48 or 72 times as long as the first hybridization time.

### 5.4.1.1 Preparing Probes for Microarrays

As noted above, the “probe” to which a particular polynucleotide molecule, such as an exon, specifically hybridizes according to the invention is a complementary polynucleotide sequence. Preferably one or more probes are selected for each target exon. For example, when a minimum number of probes are to be used for the detection of an exon, the probes normally comprise nucleotide sequences greater than 40 bases in length. Alternatively, when a large set of redundant probes is to be used for an exon, the probes normally comprise nucleotide sequences of 40-60 bases. The probes can also comprise sequences complementary to full length exons. The lengths of exons can range from less than 50 bases to more than 200 bases. Therefore, when a probe length longer than exon is to be used, it is preferable to augment the exon sequence with adjacent constitutively spliced exon sequences such that the probe sequence is complementary to the continuous mRNA fragment that contains the target exon. This will allow comparable hybridization stringency among the probes of an exon profiling array. It will be understood that each probe sequence may also comprise linker sequences in addition to the sequence that is complementary to its target sequence.

The probes can comprise DNA or DNA “mimics” (e.g., derivatives and analogues) corresponding to a portion of each exon of each gene in an organism's genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of exon segments from genomic DNA, cDNA (e.g., by RT-PCR), or cloned sequences. PCR primers are preferably chosen based on known sequence of the exons or cDNA that result in amplification of unique fragments (e.g., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14: 5399-5407; McBride et al., 1983, Tetrahedron Lett. 24: 246-248). Synthetic sequences are typically between 15 and 600 bases in length, more typically between 20 and 100 bases, most preferably between 40 and 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363: 566-568; and U.S. Pat. No. 5,539,083).

In alternative embodiments, the hybridization sites (i.e., the probes) are made from plasmid or phage clones of genes, cDNAs (e.g., expressed sequence tags), or inserts therefrom (Nguyen et al., 1995, Genomics 29: 207-209).

### 5.4.1.2 Attaching Nucleic Acids to the Solid Surface

Preformed polynucleotide probes can be deposited on a support to form the array. Alternatively, polynucleotide probes can be synthesized directly on the support to form the array. The probes are attached to a solid support or surface, which may be made, e.g., from glass, plastic (e.g., polypropylene, nylon), polyacrylamide, nitrocellulose, gel, or other porous or nonporous material.

A preferred method for attaching the nucleic acids to a surface is by printing on glass plates, as is described generally by Schena et al., 1995, Science 270: 467-470. This method is especially useful for preparing microarrays of cDNA (See also, DeRisi et al, 1996, Nature Genetics 14: 457-460; Shalon et al., 1996, Genome Res. 6: 639-645; and Schena et al., 1995, Proc. Natl. Acad. Sci. U.S.A. 93: 10539-11286).

A second preferred method for making microarrays is by making high-density polynucleotide arrays. Techniques are known for producing arrays containing thousands of oligonucleotides complementary to defined sequences, at defined locations on a surface using photolithographic techniques for synthesis in situ (see, Fodor et al., 1991, Science 251: 767-773; Lockhart et al., 1996, Nature Biotechnology 14: 1675; U.S. Pat. Nos. 5,578,832; 5,556,752; and 5,510,270) or other methods for rapid synthesis and deposition of defined oligonucleotides (Blanchard et al., Biosensors & Bioelectronics 11: 687-690). When these methods are used, oligonucleotides (e.g., 60-mers) of known sequence are synthesized directly on a surface such as a derivatized glass slide. The array produced can be redundant, with several polynucleotide molecules per exon.

Other methods for making microarrays, e.g., by masking (Maskos and Southern, 1992, Nucl. Acids. Res. 20: 1679-1684), may also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

In a particularly preferred embodiment, microarrays of the invention are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioelectronics 11: 687-690; Blanchard, 1998, in Synthetic DNA Arrays in Genetic Engineering, Vol. 20, J. K. Setlow, Ed., Plenum Press, New York at pages 111-123; and U.S. Pat. No. 6,028,189 to Blanchard. Specifically, the polynucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (e.g., the different probes). Polynucleotide probes are normally attached to the surface covalently at the 3, end of the polynucleotide. Alternatively, polynucleotide probes can be attached to the surface covalently at the 5′ end of the polynucleotide (see for example, Blanchard, 1998, in Synthetic DNA Arrays in Genetic Engineering, Vol. 20, J. K. Setlow, Ed., Plenum Press, New York at pages 111-123).

### 5.4.1.3 Target Polynucleotide Molecules

Target polynucleotides that can be analyzed by the methods and compositions of the invention include RNA molecules such as, but by no means limited to, messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides which may also be analyzed by the methods and compositions of the present invention include, but are not limited to DNA molecules such as genomic DNA molecules, cDNA molecules, and fragments thereof including oligonucleotides, ESTs, STSs, etc.

The target polynucleotides can be from any source. For example, the target polynucleotide molecules may be naturally occurring nucleic acid molecules such as genomic or extragenomic DNA molecules isolated from an organism, or RNA molecules, such as mRNA molecules, isolated from an organism. Alternatively, the polynucleotide molecules may be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In preferred embodiments, the target polynucleotides of the invention will correspond to particular genes or to particular gene transcripts (e.g., to particular mRNA sequences expressed in cells or to particular cDNA sequences derived from such mRNA sequences). However, in many embodiments, particularly those embodiments wherein the polynucleotide molecules are derived from mammalian cells, the target polynucleotides may correspond to particular fragments of a gene transcript. For example, the target polynucleotides may correspond to different exons of the same gene, e.g., so that different splice variants of that gene may be detected and/or analyzed.

In preferred embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation and an oligo dT purification (Chirgwin et al., 1979, Biochemistry 18: 5294-5299). In another embodiment, RNA is extracted from cells using guanidinium thiocyanate lysis followed by purification on RNeasy columns (Qiagen). cDNA is then synthesized from the purified mRNA using, e.g., oligo-dT or random primers. In preferred embodiments, the target polynucleotides are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. Pat. No. 6,271,002, and U.S. Provisional Patent Application Ser. No. 60/253,641, filed on Nov. 28, 2000, by Ziman et al.). Both oligo-dT primers (U.S. Pat. Nos. 5,545,522 and 6,132,997) or random primers (U.S. Provisional Patent Application Ser. No. 60/253,641, filed on Nov. 28, 2000, by Ziman et al.) that contain an RNA polymerase promoter or complement thereof can be used. Preferably, the target polynucleotides are short and/or fragmented polynucleotide molecules which are representative of the original nucleic acid population of the cell.

The target polynucleotides to be analyzed by the methods and compositions of the invention are preferably detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

Preferably, the detectable label is a fluorescent label, e.g., by incorporation of nucleotide analogs. Other labels suitable for use in the present invention include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include ³²P, ³⁵S, ¹⁴C, ¹⁵N and ¹²⁵I. Fluorescent molecules suitable for the present invention include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41. Fluorescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in less preferred embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

### 5.4.1.4 Hybridization to Microarrays

As described supra, nucleic acid hybridization and wash conditions are chosen so that the polynucleotide molecules to be analyzed by the invention (referred to herein as the “target polynucleotide molecules) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93: 10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5% sodium Sarcosine and 30 percent formamide.

### 5.4.1.5 Signal Detection and Data Analysis

It will be appreciated that when target sequences, e.g., cDNA or cRNA, complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (i.e., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In preferred embodiments, target sequences, e.g., cDNAs or cRNAs, from two different cells are hybridized to the binding sites of the microarray. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA or cRNA derived from each of the two cell types are differently labeled so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, changes the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Science 270: 467-470, which is incorporated by reference in its entirety for all purposes. The scheme is equally applicable to labeling and detection of exons. An advantage of using target sequences, e.g., cDNAs or cRNAs, labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6: 639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6: 639-645. Alternatively, the fiber-optic bundle described by Ferguson et al., 1996, Nature Biotech. 14: 1681-1684, may be used to monitor mRNA abundance levels at a large number of sites simultaneously.

Signals are recorded and, in a preferred embodiment, analyzed by computer, e.g., using a 12 bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA and/or an exon expressed in an mRNA in two cells or cell lines is scored as perturbed (i.e., the abundance is different in the two sources of mRNA tested) or as not perturbed (i.e., the relative abundance is the same). As used herein, a difference between the two sources of RNA of at least a factor of 25 percent (e.g., RNA is 25 more abundant in one source than in the other source), more usually 50 percent, even more often by a factor of 2 (e.g., twice as abundant), 3 (three times as abundant), or 5 (five times as abundant) is scored as a perturbation. Present detection methods allow reliable detection of differences of an order of 1.5 fold to 3-fold.

It is, however, also advantageous to determine the magnitude of the relative difference in abundances for an mRNA and/or an exon expressed in an mRNA in two cells or in two cell lines. This can be carried out, as noted above, by calculating the ratio of the emission of the two fluorophores used for differential labeling, or by analogous methods that will be readily apparent to those of skill in the art.

### 5.4.2 Other Methods of Transcriptional State Measurement

The transcriptional state of a cell can be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93: 659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270: 484-487).

The transcriptional state of a cell can also be measured by reverse transcription-polymerase chain reaction (RT-PCR). RT-PCR is a technique for mRNA detection and quantitation. RT-PCR is sensitive enough to enable quantitation of RNA from a single cell. See, for example, Pfaffl and Hageleit, 2001, Biotechnology Letters 23, 275-282; Tadesse et al., 2003, Mol Genet Genomics 269, p. 789-796; and Kabir and Shimizu, 2003, J. Biotech. 9, p. 105. To measure gene expression using RT-PCR, the mRNA is first reverse-transcribed into cDNA, and the cDNA is then amplified to measurable levels using PCR. Using built-in calibration techniques, RT-PCR can achieve high accuracy coupled with a sensitivity of 10 molecules/10 microliters assay volume and a dynamic range covering 6-8 orders of magnitude.

The transcriptional state of a cell can also be measured by Serial Analysis of Gene Expression (SAGE). First, double stranded cDNA is created from the mRNA. A single ten base pair (long enough to uniquely identify each gene) “sequence tag” is cut from a specific location in each cDNA. Then the sequence tags are concatenated into a long double stranded DNA that can then be amplified and sequenced. See, for example, Velculesco et al., 1997, Cell 88, p. 243-251; Zhang, 1997, Science 276, p. 1268-1272; and Polyak, 1997, Nature 389, p. 300-305.

### 5.5 Measurement of Other Aspects of the Biological State

In various embodiments of the present invention, aspects of the biological state other than the transcriptional state, such as the translational state, the activity state, or mixed aspects can be measured. Thus, in such embodiments, cellular constituent abundance data can include translational state measurements or even protein expression measurements. Details of embodiments in which aspects of the biological state other than the transcriptional state are described in this section.

### 5.5.1 Translational State Measurements

Measurement of the translational state can be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93: 1440-1445; Sagliocco et al., 1996, Yeast 12: 1519-1533; Lander, 1996, Science 274: 536-539. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

### 5.5.2 Other Types of Cellular Constituent Characteristic Measurements

The methods of the invention are applicable to any cellular constituent that can be monitored. For example, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In some embodiments of the present invention, cellular constituent measurements are derived from cellular phenotypic techniques. One such cellular phenotypic technique uses cell respiration as a universal reporter. In one embodiment, 96-well microtiter plate, in which each well contains its own unique chemistry is provided. Each unique chemistry is designed to test a particular phenotype. Cells from the organism of interest are pipetted into each well. If the cells exhibits the appropriate phenotype, they will respire and actively reduce a tetrazolium dye, forming a strong purple color. A weak phenotype results in a lighter color. No color means that the cells don't have the specific phenotype. Color changes can be recorded as often as several times each hour. During one incubation, more than 5,000 phenotypes can be tested. See, for example, Bochner et al., 2001, Genome Research 11, p. 1246.

In some embodiments of the present invention, cellular constituent measurements are derived from cellular phenotypic techniques. One such cellular phenotypic technique uses cell respiration as a universal reporter. In one embodiment, 96-well microtiter plates, in which each well contains its own unique chemistry is provided. Each unique chemistry is designed to test a particular phenotype. Cells from biological specimens of interest are pipetted into each well. If the cells exhibit the appropriate phenotype, they will respire and actively reduce a tetrazolium dye, forming a strong purple color. A weak phenotype results in a lighter color. No color means that the cells don't have the specific phenotype. Color changes may be recorded as often as several times each hour. During one incubation, more than 5,000 phenotypes can be tested. See, for example, Bochner et al., 2001, Genome Research 11, 1246-55.

In some embodiments of the present invention, the cellular constituents that are measured are metabolites. Metabolites include, but are not limited to, amino acids, metals, soluble sugars, sugar phosphates, and complex carbohydrates. Such metabolites can be measured, for example, at the whole-cell level using methods such as pyrolysis mass spectrometry (Irwin, 1982, Analytical Pyrolysis: A Comprehensive Guide, Marcel Dekker, New York; Meuzelaar et al., 1982, Pyrolysis Mass Spectrometry of Recent and Fossil Biomaterials, Elsevier, Amsterdam), fourier-transform infrared spectrometry (Griffiths and de Haseth, 1986, Fourier transform infrared spectrometry, John Wiley, New York; Helm et al., 1991, J. Gen. Microbiol. 137, 69-79; Naumann et al., 1991, Nature 351, 81-82; Naumann et al., 1991, In: Modern techniques for rapid microbiological analysis, 43-96, Nelson, W. H., ed., VCH Publishers, New York), Raman spectrometry, gas chromatography-mass spectroscopy (GC-MS) (Fiehn et al., 2000, Nature Biotechnology 18, 1157-1161, capillary electrophoresis (CE)/MS, high pressure liquid chromatography/mass spectroscopy (HPLC/MS), as well as liquid chromatography (LC)-Electrospray and cap-LC-tandem-electrospray mass spectrometries. Such methods can be combined with established chemometric methods that make use of artificial neural networks and genetic programming in order to discriminate between closely related samples.

### 5.6 Analytic Kit Implementation

In one embodiment, the methods of this invention can be implemented by use of kits for developing and using biological classifiers. Such kits contain microarrays, such as those described in Subsections above. The microarrays contained in such kits comprise a solid phase, e.g., a surface, to which probes are hybridized or bound at a known location of the solid phase. Preferably, these probes consist of nucleic acids of known, different sequence, with each nucleic acid being capable of hybridizing to an RNA species or to a cDNA species derived therefrom. In a particular embodiment, the probes contained in the kits of this invention are nucleic acids capable of hybridizing specifically to nucleic acid sequences derived from RNA species in cells collected from an organism of interest.

In a preferred embodiment, a kit of the invention also contains one or more data structures and/or software modules described above and in FIGS. 1 and/or 4, encoded on computer readable medium, and/or an access authorization to use the databases described above from a remote networked computer.

In another preferred embodiment, a kit of the invention contains software capable of being loaded into the memory of a computer system such as the one described supra, and illustrated in FIG. 1. The software contained in the kit of this invention, is essentially identical to the software described above in conjunction with FIG. 1.

Alternative kits for implementing the analytic methods of this invention will be apparent to one of skill in the art and are intended to be comprehended within the accompanying claims.

### 5.7 Comparing Models

It is sometimes desirable to be able to rank models (sets 72) and to be able to say than one model (set 72) is superior to another. A model with a higher fraction of correct classifications and a lower or equal fraction of incorrect classifications, and a lower or equal fraction of indeterminate classifications is a superior model. However it is often the case that the results of a comparison is not so clear. In the latter case the present invention assigns a utility function to each of the possible outcomes of the classification. Thus, a value (or cost) is assigned to each of the possible outcomes of the classification and the expected value (cost) of a classification is used as the value (cost) of a model, and one can say that a model with a higher value (lower cost) is a superior model.

In the most usual case, a value is assigned to a correct classification Value(Correct), another (lower) to an indeterminate classification Value(Indeterminate), and yet another (even lower) to an incorrect classification Value(Incorrect). In this case the value of a model can be computed as:
Value=Correct*Value(Correct)+Indeterminate*Value(Indeierminate)+Incorrect*Value(Incorrect)
Note that it is possible in the computation of Value (Cost) to have a more detailed description of the values (costs) of individual classifications. For example, not all incorrect classifications are equally costly.

### 5.8 Validating Models

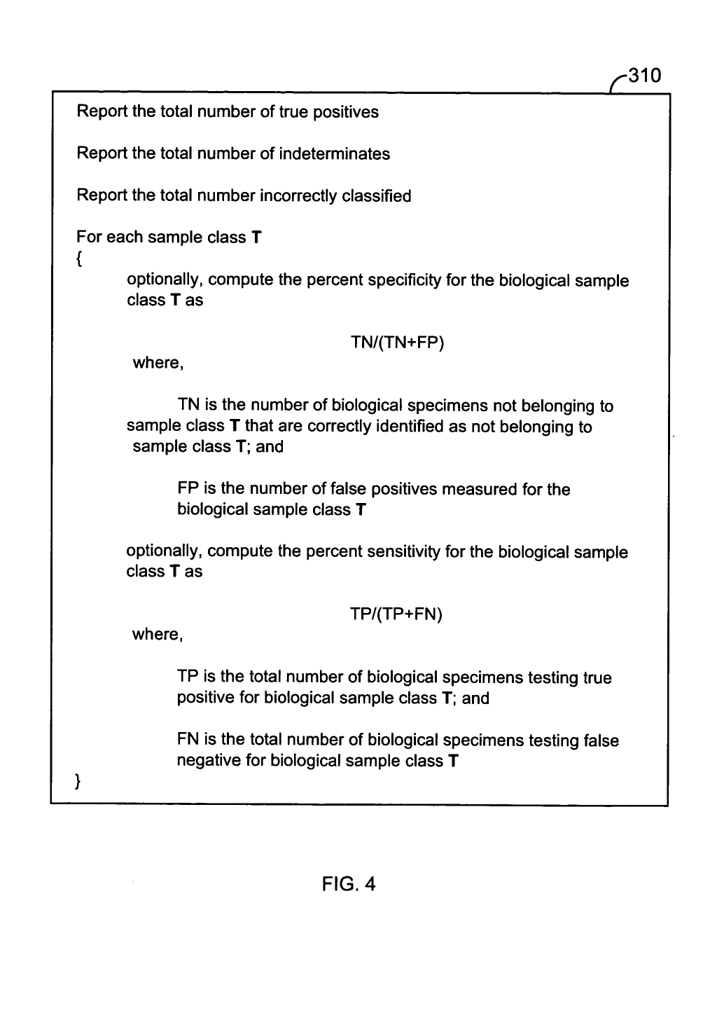

Methods for creating sets 72 (models) have been described in Section 5.1 above. In some embodiments, such methods are validated by using the methods described in Section 5.2 with a plurality of biological specimens having known biological sample classification. In other words, a plurality of biological specimens of known classification are tested using the steps outlined in FIG. 3 in order to test the quality of the classifiers (the sets 72). Then, certain statistics can be computed. Step 310 (FIG. 4) outlines some representative statistics that can be computed in such instances. In some embodiments of the present invention, step 310 is performed by model statistical report module 78.

In the embodiment of step 310 illustrated in FIG. 4, the total number of true positives, indeterminates, and incorrectly classified biological specimens in the plurality of biological specimens are specified. Next, for each biological sample class T considered, the percent specificity of the biological sample class is considered as:
TN/(TN+FP)
where
TN is the number of biological specimens not belonging to sample class T that are correctly identified as not belonging to class T; and
FP is the number of false positives measured for the sample class T, where false positive is as defined in step 308 above.

Further, for each biological sample class T considered, the percent sensitivity of the biological sample class is considered as:
TP/(TP+FN)
where
TP is the total number of biological specimens testing true positive for the biological sample class T; and
FN is the total number of specimens testing false negative for the biological sample class T.

In other embodiments of step 310, the plurality of biological specimens with known classification are run through the methods described in Section 5.2 and then analyzed according to the following truth table:

| | | Truth | | |
|---|---|---|---|---|
| | | Feat. 1 | Feat. 2 | Feat. 3 |
| | | Present | Present | Present |
| Prediction | Feat. 1 Present | Correct (1) | Incorrect | Incorrect |
| | | | (1, 2) | (1, 3) |
| | Feat. 2 Present | Incorrect | Correct (2) | Incorrect |
| | | (2, 1) | | (2, 3) |
| | Feat. 3 Present | Incorrect | Incorrect | Correct (3) |
| | | (3, 1) | (3, 2) | |
| | Indetermined | Incon- | Incon- | Incon- |
| | | clusive (1) | clusive (2) | clusive (3 |

The total number of samples can be computed by adding all possible classifications:

$$total = {\sum }_{i = 1}^{n} Correct ( i ) + {\sum }_{i = 1}^{n} {\sum }_{{j = 1}_{j \neq i}}^{n} Incorrect ( i , j ) + {\sum }_{i = 1}^{n} Indeterminate ( i )$$

Fraction of samples correctly identified:

$$Correct = \frac{{\sum }_{i = 1}^{n} Correct ( i )}{total} \qquad ( I )$$

Fraction of samples incorrectly identified:

$$Incorrect = \frac{{\sum }_{i = 1}^{n} {\sum }_{{j = 1}_{j \neq i}}^{n} Incorrect ( i , j )}{total} \qquad ( II )$$

Fraction of samples for which the test offered inconclusive results and were not identified:

$$Indeterminate = \frac{{\sum }_{i = 1}^{n} Indeterminate ( i )}{total} \qquad ( III )$$

Example where this embodiment of step 310 is used are described in the Examples Section below.

### 5.9 Receiver Operating Characteristic Curve Embodiments

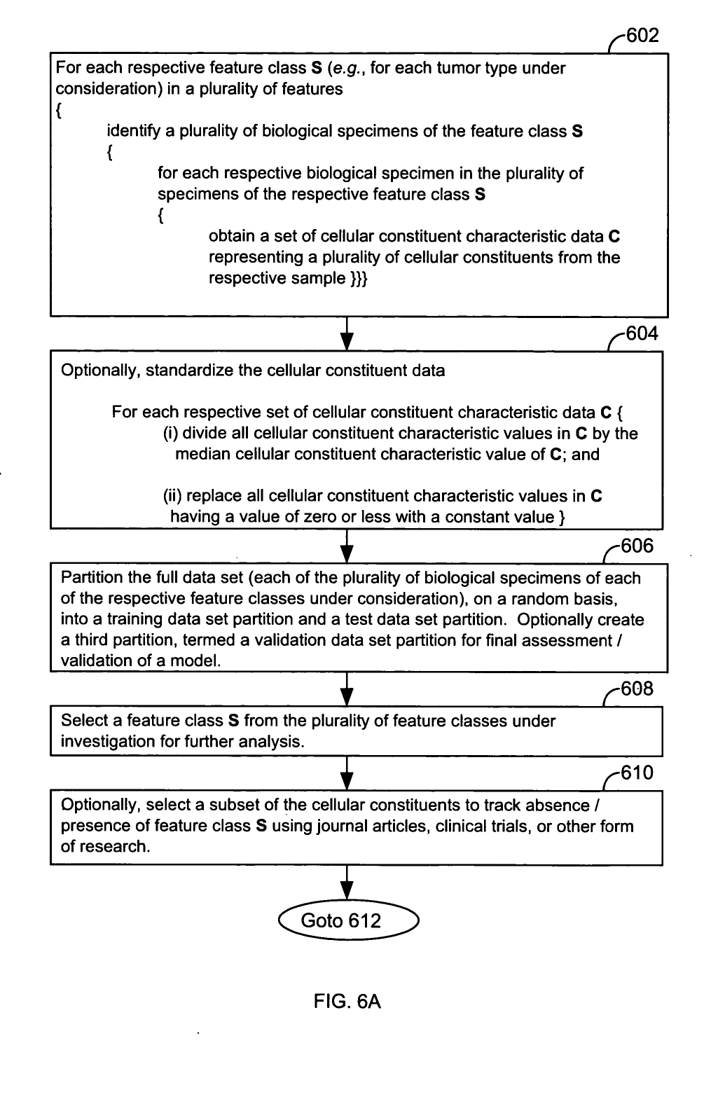

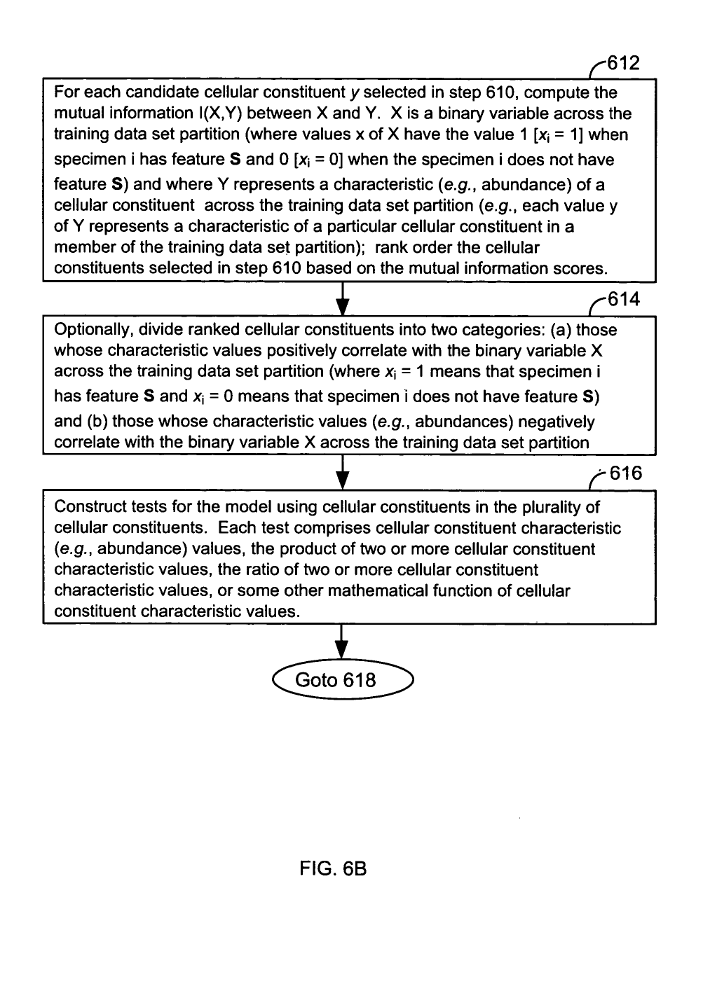

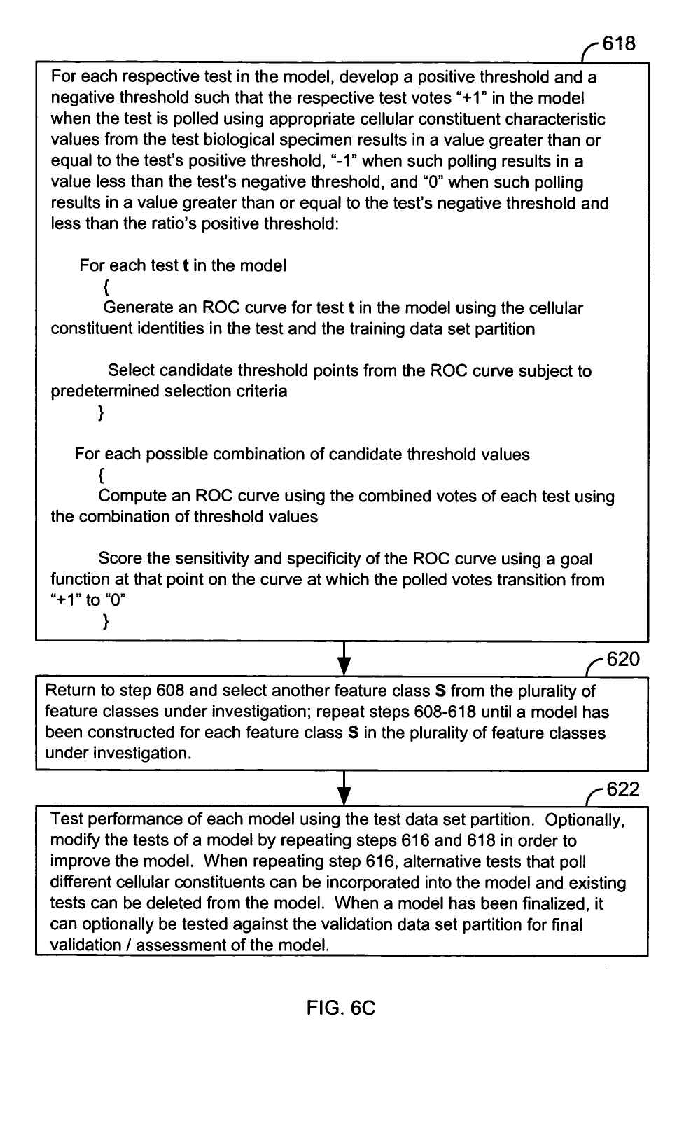

This section describes processing steps that are performed to create models in accordance with another aspect of the present invention. In some instances, such steps are performed by model creation application 61 (FIG. 1). The overall process flow of the embodiments described in this section is illustrated in FIG. 6.

Step 602.

In step 602, cellular constituent characteristic data is obtained for each respective feature class S in a plurality of feature classes to be distinguished. In some embodiments, a feature is a tumor type and a feature class S are those biological specimens that have a given tumor type. For each respective feature class S in a plurality of feature classes, a plurality of biological specimens of the feature class is identified. For each respective biological specimen B in the plurality of biological specimens of a given feature class, a set of cellular constituent characteristic data representing a plurality of cellular constituents from the respective biological specimen B is obtained. This obtaining is repeated for each feature class in the plurality of feature classes so that there is cellular constituent characteristic data for each feature class.

In some embodiments, cellular constituent characteristic data represents amounts (e.g., gene expression level, amounts of protein) of cellular constituents in biological specimens. In other embodiments, cellular constituent characteristic data represents a cellular constituent state. An example of a cellular constituent state is the degree of phosphorylation or methylation.

As described above, in step 602, cellular constituent characteristic data 60 (e.g., from a gene expression study, proteomics study, etc.) is obtained for a plurality of cellular constituents from one or more members of each feature class under study. In some embodiments, the set of cellular constituent characteristic data 60 obtained from a corresponding biological specimen 58 comprises the processed microarray image for the specimen. For example, in one such embodiment, such data comprises cellular constituent characteristic information for each cellular constituent represented on the array, optional background signal information, and optional associated annotation information describing the probe used for the respective cellular constituent.

In some embodiments, cellular constituent characteristic measurements are transcriptional state measurements as described in Section 5.4, above. In various embodiments of the present invention, aspects of the biological state other than the transcriptional state, such as the translational state, the activity state, or mixed aspects can be measured and used as cellular constituent characteristic data. See, for example, Section 5.5, above. For instance, in some embodiments, cellular constituent characteristic data 60 is, in fact, protein levels for various proteins in the biological specimens under study for which cellular constituent characteristic data is measured. Thus, in some embodiments, cellular constituent characteristic data comprises amounts or concentrations of the cellular constituent in tissues of the organisms under study, cellular constituent activity levels in one or more tissues of the organisms under study, the state of cellular constituent modification (e.g., phosphorylation), or other measurements relevant to the trait under study.

In some embodiments, cellular constituent characteristic data 60 is taken from tissues that have been associated with the corresponding biological sample class 56. For example, in the case of tumor of unknown primary origin, each biological specimen corresponds to a primary tumor from a known origin.

Step 604.

In step 604 cellular constituent data 60 is optionally standardized. In some instances, standardization module 62 of model creation application 61 is used to perform this standardization. In some embodiments, for each respective set of cellular constituent data 60, all cellular constituent characteristic values in the set are divided by the median cellular constituent characteristic value of the set.

In the case where the source of the cellular constituent characteristic measurements is a microarray, negative cellular constituent characteristic values can be obtained when a mismatched probe measure is greater than a perfect match probe. This typically occurs when the primary gene (representing a cellular constituent) is expressed at low levels. In some representative cases, on the order of thirty percent of the characteristic values in a given cellular constituent characteristic dataset 60 are negative. In some embodiments of the present invention, all cellular constituent characteristic values in datasets 60 with a value of zero or less are replaced with a fixed value. In the case where the source of the cellular constituent characteristic measurements is an Affymetrix GeneChip MAS 4.0, negative cellular constituent characteristic values can be replaced with a fixed value such as 20 or 100 in some embodiments. More generally, in some embodiments, all cellular constituent characteristic values in datasets 60 with a value of zero or less can be replaced with a fixed value that is between 0.001 and 0.5 (e.g., 0.1 or 0.01) of the median cellular constituent characteristic value of the set of cellular constituent characteristic data 60.

In some embodiments, standardization of cellular constituent abundances comprises dividing by the median of a subset of cellular constituents known to be particularly stable across specimens (e.g., housekeeping cellular constituents). In some embodiments, there are between five and 100 housing keeping cellular constituents, between twenty and 1000 housing keeping cellular constituents, more then two housing keeping cellular constituents, more then fifty housing keeping cellular constituents, or more than one hundred house keeping cellular constituents.

Step 606.

The source cellular constituent data collected in step 602 can be considered an n by m matrix where n is the number of biological samples tested and m is the number of cellular constituents for which cellular constituent characteristic data is measured. However, there is no requirement that cellular constituent characteristic data for each of the m cellular constituents be measured in each of the biological specimens. Further, there is no requirement that cellular constituent characteristic data for each of n biological samples be measured in the same study. Cellular constituent data from any number of studies, performed at any number of laboratories, can be combined to form the n by m matrix.

In step 606, the n by m matrix is partitioned, on a random basis, into three partitions:
(i) a training data set partition, (ii) a test data set partition, and (iii) a validation data set partition. Each partition includes cellular constituent characteristic data for the full set of m cellular constituents. However, each of the partitions has only a unique subset of the n biological samples. To illustrate, consider the case in which cellular constituent data from fifty biological samples (e.g., tumors) is obtained in a first study and cellular constituent data from one hundred biological samples is obtained in a second study. First, the two studies are combined to form the n by m matrix, where n is 150. Next, the n by m matrix is partitioned into (i) a training data set partition that includes cellular constituent data for 50 specimens randomly chosen from the n by m matrix (randomly chosen from specimens tested in the first and the second study), (ii) a test data set partition that includes cellular constituent data for 50 specimens randomly chosen from the n by m matrix with the proviso that such specimens are not found in the training data set partition, and (iii) a validation data set partition that includes the remaining 50 specimens. Although each partition received an equal number of specimens in this example, in practice, there is no requirement that each of the partitions be allocated an equal or near equal number of specimens. In fact, there is no restriction on the percentage of the total number of specimens represented by the n by m matrix that can be allocated to each partition so long as each partition is allocated specimens that are not allocated to any of the other partitions. In some embodiments, the n by m matrix is divided into only two partitions, a training data set partition and a test data set partition.

In preferred embodiments of step 606, the data that is partitioned into the training, test, and validation partitions is all data, regardless of feature class. In other words, the data measured for each of the feature classes under consideration is combined and then divided into the respective partitions.

Step 608.

In step 608, a feature class S from the plurality of feature classes under investigation is selected for further analysis.

Step 610.

In optional step 610, cellular constituents are selected for each feature class S in a plurality of feature classes to be distinguished. In some embodiments, the cellular constituent selection that occurs in step 610 uses the cellular constituents identified in a journal article or other form of research. The work of Suet al., 2001, Cancer Research 61, 7388 illustrates the point. In Su et al., the expression of 9198 genes in 100 primary carcinomas representing 11 different tumor classes (prostate, bladder/ureter, breast, colorectal, gastroesophagus, kidney, liver, ovary, pancreas, lung adenocarcinoma, and lung squamous cell carcinoma) was used to develop a classification scheme. In the first stage of this classifier development, the expression levels of the 9198 genes were pre-filtered to identify genes with uniformly high expression among carcinomas of a specific anatomical site and uniformly low expression among carcinomas of all other anatomical sites. This was achieved using a Wilcoxon rank-sum test that tests the null hypothesis that gene expression in one tumor class is not different from gene expression in any other tumor class. For each respective tumor class in the set of 11 tumor classes, a Wilcoxon rank score is computed for each of the genes having the highest mean expression in the tumor class. Each Wilcoxon rank score is calculated based upon (i) gene expression in the high expressing tumor class versus (ii) gene expression in all other tumor classes. For example, if gene 1 has very high expression in tumor class A, a Wilcoxon rank score is computed based upon (i) the expression levels of gene 1 in tumor class A versus (ii) the expression levels of gene 1 in all other tumor classes. One hundred of the Wilcoxon-selected genes from each class (the 100 genes with the lowest P-score in each class) (total, 1100) were ranked based on their predictive accuracy for discriminating one tumor class versus all others using a support vector machine classifier. Each of the 1100 genes were individually tested for their ability to discriminate one tumor class from all other tumor classes, using a support vector machine algorithm. The support vector machine test identified more then ten genes per tumor class that could predict the class of a blinded tumor in at least 91 percent of cases. Together, the more than ten genes per tumor class represented a set of 216 genes. As such the set could be considered a multiclass predictor set for each of 11 tumor classes.

The Su et al. approach represents just one approach in accordance with step 610. Other approaches in accordance with step 610 are disclosed in, for example, Bhattachaijee et al., 2001, Proceedings National Academy of Science 98, 13790; Gordon et al., 2003, Journal of the National Cancer Institute 95, 598; and Gordon et al., 2002, Cancer Research 62, 4963, to name a few.

Step 612.

The set of cellular constituents identified in step 610 is rank ordered in step 612. In some embodiments, step 610 is not performed. In such instances, each of the cellular constituents for which characteristic data was obtained for the feature class S under consideration in step 602 is rank ordered in step 612. Table 4 details the type of data available for each cellular constituent under consideration.

TABLE 4
Exemplary data for a cellular constituent to be rank
ordered in step 612

| Identity of source | Presence of feature S in | Cellular constituent |
|---|---|---|
| biological specimen | source biological specimen | characteristic |
| A001 | 1 | 115 |
| A002 | 0 | 130 |
| A003 | 1 | 197 |
| A004 | 1 | 204 |
| B001 | 0 | 70 |
| B002 | 0 | 67 |
| B003 | 1 | 150 |

As illustrated in column 3 of Table 4, for each respective cellular constituent to be rank ordered, there exists cellular constituent characteristic information for the cellular constituent from a plurality of source biological specimens. For each of these source biological specimens, there is an indication as to whether the biological specimen has the target feature (is a member of a given feature class S or not). For instance, as illustrated in column 2 in Table 4, if the biological sample has the target feature (is a member of feature class S), then the biological sample is assigned a “1”. If the biological sample does not have the target feature (is not a member of biological sample class S), then the biological sample is assigned a “0”.

Only the data from the training data set partition is used in step 612 to rank order cellular constituents. Despite this limitation, the data available for each respective cellular constituent to be rank ordered still has the data format shown in Table 4. It is simply the case that such data is from the training data set partition and therefore represents just a subset of the total data measured in step 602.

The absence or presence of a given feature, shown in column 2 of Table 4, represents a distribution p(x) (also termed p) of the binary variable x across the training data set partition for a given cellular constituent. For any given biological specimen i, a value xᵢ=1 is assigned if the specimen i has feature S and a value xᵢ=0 is assigned if the specimen i does not have feature S. The characteristic values of the given cellular constituent shown in column 3 of Table 4 represents q(y), the distribution of cellular constituent i characteristic values across the training data set partition. Each cellular constituent to be rank ordered has an associated q(y) (also termed q).

In step 612, for each respective cellular constituent under consideration, the mutual information I(X,Y) between X (the binary variable indicating presence/absence of feature S across the training data set partition) and Y (the characteristic values for a given cellular constituent y across the training data set partition) is computed. Thus, a value I(X,Y) is computed for each cellular constituent to be rank ordered. The cellular constituents are then ranked based on their associated I(X,Y) values.

The mutual information is the reduction in uncertainty about one variable X due to the knowledge of the other variable Y and can be expressed as:

$$I ( X , Y ) = H ( X ) - H ( X \vert Y ) = {\sum }_{x , y}^{} r ( x , y ) {log}_{2} \frac{r ( x , y )}{xy} \qquad Eqn . 1$$

where,
H(X) is the entropy of X;
H(X|Y) is the entropy of X given Y;
X is a binary random variable wherein each value x of X represents the presence (xᵢ=1) or absence (xᵢ=0) of feature S in a member i of the training data set partition;
Y is a random variable wherein each value y of Y represents an amount of a cellular constituent characteristic for a respective cellular constituent in a respective member of the training data set partition; and
r(x,y) is the joint distribution of X and Y.
   Mutual information is the relative entropy between the joint distribution r(x,y) and the product distribution p(x)_{q}(y) and as such it measures how much the distributions of variables differ from statistical dependence. See, for example, Duda, Pattern Classification, Second Edition, John Wiley & Sons, Inc., New York, pp. 630-633; and Shannon and Weaver, 1949, The mathematical theory of communication, University of Illinois Press, Urbana.

Mutual information is based on the assumption that the uncertainty regarding any variable Z characterized by a probability distribution P(z) can be represented by the entropy function

$$H ( Z ) = - {\sum }_{z}^{} P ( z ) log P ( z ) .$$

Accordingly, the residual uncertainty regarding the true value of the target p, given that p is instantiated to y, can be written:

$$H ( p \vert y ) = - {\sum }_{x}^{} P ( x \vert y ) log P ( x \vert y ) ,$$

and the average residual uncertainty in p (the distribution of the binary variable x-absence or presence of feature S—across the training data set partition), summed over all possible outcomes y (cellular constituent characteristic values for a respective cellular constituent i in the training data set partition), is

$$H ( p \vert q ) = {\sum }_{x}^{} H ( p \vert y ) P ( y ) = - {\sum }_{y}^{} {\sum }_{x}^{} P ( x , y ) P ( x \vert y ) .$$

If H(p|q) is subtracted from the original uncertainty for p prior to consulting q, namely H(p), the total uncertainty-reducing potential of q (the distribution of cellular constituent characteristic values for a respective cellular constituent in the training data set) is realized. This potential is called Shannon's mutual information and is given by

$$I ( p ; q ) = H ( p ) - H ( p \vert q ) = - {\sum }_{y}^{} {\sum }_{xt}^{} P ( x , y ) log \frac{r ( x , y )}{P ( x ) P ( y )} . \qquad Eqn . 2$$

See also, Pearl, 1988, Probabilistic Reasoning In Intelligent Systems: Networks of plausible Inference, revised second printing, Morgan Kaufinann, Publishers, San Francisco, Calif., pp. 321-323.

Step 614.

In step 614 a determination is made, for each respective cellular constituent ranked in step 612, as to whether there is a positive or negative correlation between the q(y) associated with the respective cellular constituent and p(x) (the distribution of the binary variable x—absence or presence of feature S across the training data set partition). Then the cellular constituents under consideration are divided into two categories: (a) those cellular constituents in which the associated q(y) and p(x) are positively correlated and (b) those cellular constituents in which the associated q(y) and p(x) are negatively correlated. In other words, in step 614, the cellular constituents ranked in step 612 are divided into two categories, (a) those cellular constituents whose characteristic values are positively correlated with the absence or presence of feature S in the training data set partition and (b) those cellular constituents whose characteristic values are negatively correlated with the absence or presence of feature S in the training data set partition.

A correlation describes the strength of an association between variables. An association between variables means that the value of one variable can be predicted, to some extent, by the value of the other. For a set of variable pairs (cellular constituent characteristic values versus absence or presence of a feature S), the correlation coefficient gives the strength of the association. The square of the size of the correlation coefficient is the fraction of the variance of the one variable that can be explained from the variance of the other variable. The relation between the variables is termed the regression line. The regression line is defined as the best fitting straight line through all value pairs, e.g., the one explaining the largest part of the variance. The correlation coefficient is calculated with the assumption that both variables are stochastic (i.e., bivariate Gaussian). See for example, Smith, Statistical Reasoning, 1991, Allyn and Bacon, Boston Mass. The correlation coefficient can range from −1 to 1.

Step 616.

In step 616, cellular constituents are selected to form a plurality of tests for prediction of the absence or presence of feature S in a test biological specimen. This plurality of tests is referred to as a model. The cellular constituents used to form tests in step 616 are those cellular constituents that ranked highly in step 612.

In preferred embodiments, each test comprises a ratio between the characteristic (e.g., abundance) of a first cellular constituent and a second cellular constituent. Those highly ranked cellular constituents whose characteristic values are positively correlated with X are used as numerators while those highly ranked cellular constituents whose characteristic values are negatively correlated with X are used as denominators in such ratios. As an example, consider the case in which cellular constituents A, B, C, D, E, and F rank highly in step 612 and that characteristic values for A, B, C in the training data set partition are positively correlated with X while the characteristic values for D, E, and F are negatively correlated with X. Then, suitable candidate ratios for the model could be A/D, B/E, and C/F.

Ratios in which a single cellular constituent serves as the numerator and a single cellular constituent serves as the denominator, such as those in the example described above, serve as tests in preferred models. However, there is no absolute requirement that such ratios include, as numerators, cellular constituents whose characteristic values are positively correlated with p(x) and denominators whose characteristic values are negatively correlated with p(x). In fact, in some embodiments, step 614 is not performed. Furthermore, the invention is not limited to simple ratios. Ratios in which the numerator and/or denominator is the product of two or more cellular constituents are used in some embodiments.

In some alternative embodiments, the tests used in a model are not ratios. In such alternative embodiments, the tests used in a model for the prediction of the absence or presence of feature S in a test biological specimen can be the cellular constituent characteristic levels of highly ranked cellular constituents from step 612. For example, the model can comprise the cellular constituent characteristic values for cellular constituents A, B, and C. Alternatively, the tests used in a model for the prediction of the absence or presence of feature S in a test biological specimen can be the products of specific cellular constituent characteristic levels of highly ranked cellular constituents from step 612. For example, the model can comprise the tests A×B, C×D, and E×F.

In preferred embodiments, each test in a model uses cellular constituent characteristic values that were not used in any other test in the model. However, the invention is not limited to such embodiments. In fact, in some instances, a test (e.g., a ratio of cellular constituents, the product of two or more cellular constituents, etc.) in a model may use one or more cellular constituents that were used in other tests in the model.

Step 618.

As was the case for the embodiment illustrated in FIG. 2, each test (e.g., ratio) in a model will contribute one vote to a model. In step 618, a positive and a negative threshold is assigned to each test. In the case where the test is a ratio between the characteristic level of two cellular constituents, the test will vote “+1” if the ratio of the numerator post standardization (step 604) divided by the denominator post standardization is greater than or equal to the ratio's positive threshold. More generally, the test will vote “+1” when computation of the test using the cellular constituent characteristic values from the test biological specimen dictated by the test results in a value that is greater than or equal to the test's positive threshold.

In the case where the test is a ratio between the characteristic level of two cellular constituents, the test will vote “−1” if the ratio of the numerator post standardization (step 604) divided by the denominator post standardization is less than the ratio's negative threshold. More generally, the test will vote “−1” when computation of the test using the cellular constituent characteristic values from the test biological specimen dictated by the test results in a value that is less than the test's negative threshold.

In the case where the test is a ratio between the characteristic levels (.e.g., abundance levels) of two cellular constituents, the test will vote “0” if the ratio of the numerator post standardization (step 604) divided by the denominator post standardization is greater than or equal to the ratio's negative threshold and less than the ratio's positive threshold. More generally, the test will vote “0” when computation of the test using the cellular constituent characteristic values from the test biological specimen dictated by the test results in a value that is greater than or equal to the test's negative threshold and less than the test's positive threshold.

In step 618 the goal in assignment of positive and negative thresholds to tests in a model is to train the model so that it will cause most of the biological specimens in the training data set partition that have feature S (e.g., a particular type of cancer) to have a positive outcome and most of the biological specimens in the training data set partition that do not have feature S to have a negative outcome when polled by the model. Robust solutions to this problem are sought so that this relationship holds true not only for the training data set for also for untested organisms as well.

One aspect of the invention provides robust solutions to the problem of assigning negative and positive thresholds to the tests of a model using Receiver Operating Characteristic (ROC) curves. ROC curves are generally discussed in Park et al., Korean J. Radiol. 5, p. 11. In one embodiment of the present invention, an ROC curve is computed for each test in the model using the training data set partition. As noted in step 612, the training data set partition includes cellular constituent characteristic values for the training population and, for each specimen/organism in the training population, an indication as to whether or not the specimen/organism has the feature S under study.

Each respective ROC curve graphs the correlation between (i) the test values across the training population for the test corresponding to the respective ROC curve versus (ii) a binary indication of the presence or absence of feature S in biological specimens/organisms in the training data set partition. For example, consider the case in which there is a model for feature S that includes the ratio [characteristic of cellular constituent A]/[characteristic of cellular constituent B]. The training data provides the information found in Table 5.

TABLE 5
Values for a test in a model for feature S using data
from the training set

| [Cellular constituent A]/ | |
|---|---|
| [Cellular constituent B] | Presence/Absence of Feature S |
| 453 | Y |
| 437 | Y |
| 424 | Y |
| 374 | Y |
| 202 | N |
| 158 | Y |
| 102 | N |
| 37 | N |
| 0.54 | N |

In Table 5, each line represents a different organism and/or biological specimen in the training data set partition. If the correlation between [Cellular constituent A]/[Cellular constituent B] (characteristic of cellular constituent A divided by characteristic of cellular constituent B) and the presence of feature S in the training data set partition were perfect, all positive result (where organisms/biological specimens have feature S) would be at the top of Table 5 and all negative results (where organisms/biological specimens do not have features S) at the bottom of the Table 5.

To plot the ROC curve corresponding to the test illustrated in Table 5, the table is divided into a number of cutoff levels. Then, the sensitivity and specificity of each cutoff level is computed. Sensitivity and specificity are defined with reference to the decision matrix of Table 6.

TABLE 6
Decision matrix

| | True Condition Status | | |
|---|---|---|---|
| Test result | Positive | Negative | Total |
| Positive | TP | FP | T+ |
| Negative | FN | TN | T− |
| Total | D+ | D− | |

In Table 6, TP means the number of true positives, FT means the number of false positives, FN means the number of false negatives, and TN means the number of true negatives.

Sensitivity is the proportion of patients with feature S who test positive for the feature. In probability notation sensitivity is P(T⁺|D⁺)=TP/(TP+FN). Specificity is the proportion of patients without feature S who test negative for the feature. In probability notation specificity is P(T⁻|D⁻)=TN/(TN+FP).

The ROC curve is defined as a plot of the sensitivity as the y-coordinate versus 1-specificity (false positive rate) as the x-coordinate. Thus, for Table 5, where each line of the Table 5 represents an independent cutoff level, the following ROC data points are derived.

TABLE 7
ROC data points for Table 5

| Ratio Cutoff Level | Sensitivity | 1-Specificity |
|---|---|---|
| No row | 0 | 0 |
| First row | 0.2 | 0 |
| First two rows | 0.4 | 0 |
| First three rows | 0.6 | 0 |
| First four rows | 0.8 | 0 |
| First five rows | 0.8 | 0.25 |
| First six rows | 1 | 0.25 |
| First seven rows | 1 | 0.5 |
| First eight rows | 1 | 0.75 |
| First nine rows | 1 | 1 |

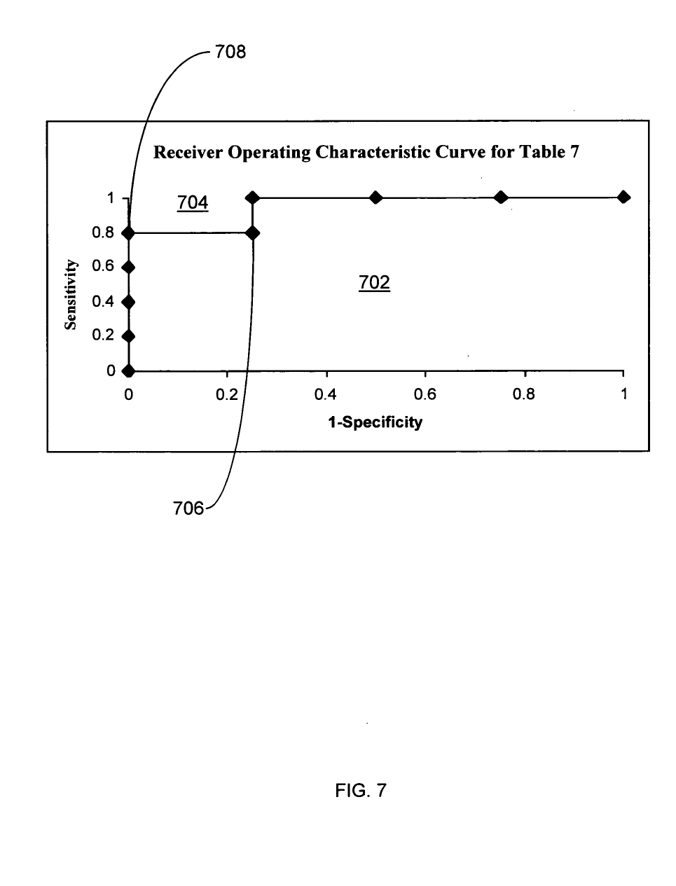

To compute the last row of Table 7, the number of TP, FP, FN, and TN are counted in Table 5 when the condition is imposed that the model predicts that no organism/specimen in Table 5 is positive for feature S. This, of course, is not an accurate model as reflected in the respective sensitivity and specificity values of 0 and 1. Plotting sensitivity by 1-specificity yields the coordinate (0,0) as illustrated in the last row of Table 7. FIG. 7 illustrates the ROC curve based upon the data points illustrated in FIG. 7. As illustrated in FIG. 7, an ROC curve begins at coordinate (0,0) and ends at coordinate (1,1).

Once an ROC curve has been computed for a given test, the curve is used to identify candidate upper threshold p^{thres} and lower threshold n^{thres} values. In one embodiment, candidate upper threshold p^{thres} and lower threshold n^{thres} values must satisfy the conditions that (i) p^{thres} and n^{thres} are points in a convex set of values where each value in the convex set is tangent to the inside of the ROC curve, and (ii) p^{thres}−n^{thres} is greater than a predetermined value, such as 0.3, 0.5, etc. The inside of an ROC curve is the area underneath the ROC curve. For example, in FIG. 7, the inside of the curve is denoted as area 702 and the outside of the ROC curve is denoted 704. In the example provided above, these conditions require that the cutoff ratio that defines p^{thres} (e.g., a specific ratio between cellular constituent A characteristic level and cellular constituent B characteristic level) must be a value such as 0.3 greater than n^{thres}.

There are many known mathematical methods for finding a convex set. See, for example, Croft et al., Convexity, 1994, Springer-Verlag, New York, pp. 6-47; Klee, 1971, Amer. Math. Monthly 78, pp. 616-631; Lay, Convex Sets and Their Applications, 1979, Wiley, New York; and Valentine, Convex Sets, 1964, McGraw-Hill, New York. To be in the convex set described above, a point must mark a place where the ROC curve goes from horizontal to vertical when going from left to right. In FIG. 7, point 706 marks such a point that is in the convex set. The ROC curve is horizontal to the left of point 706 and vertical to the right of point 706.

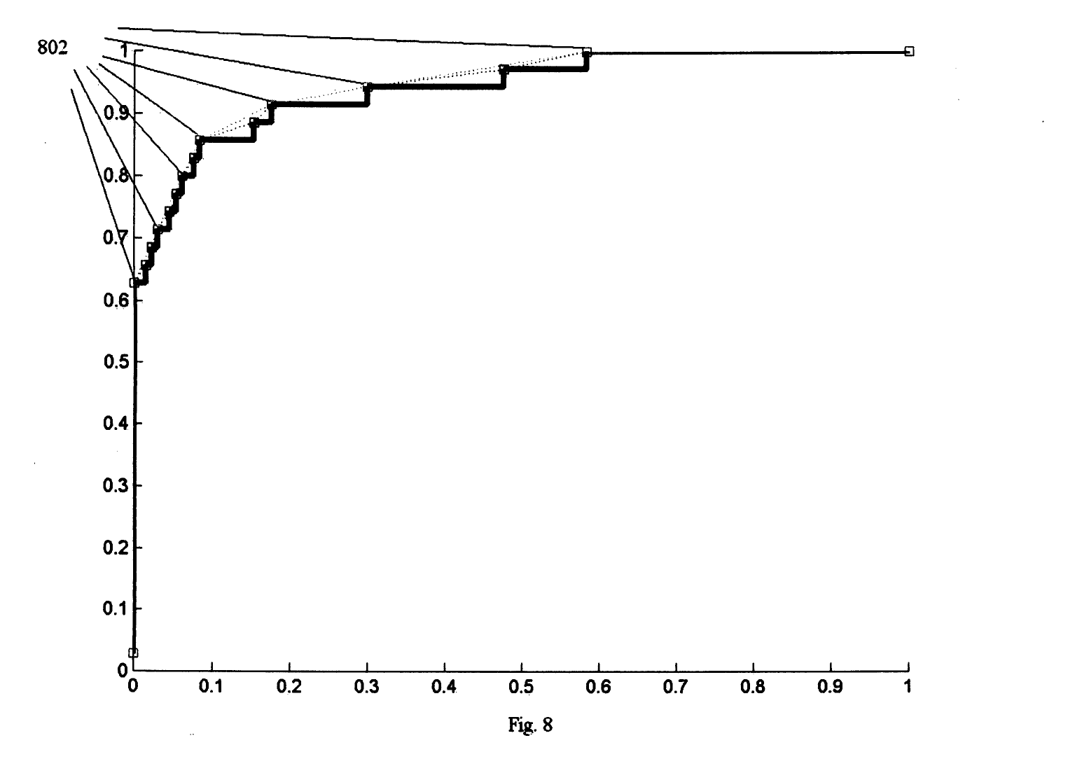

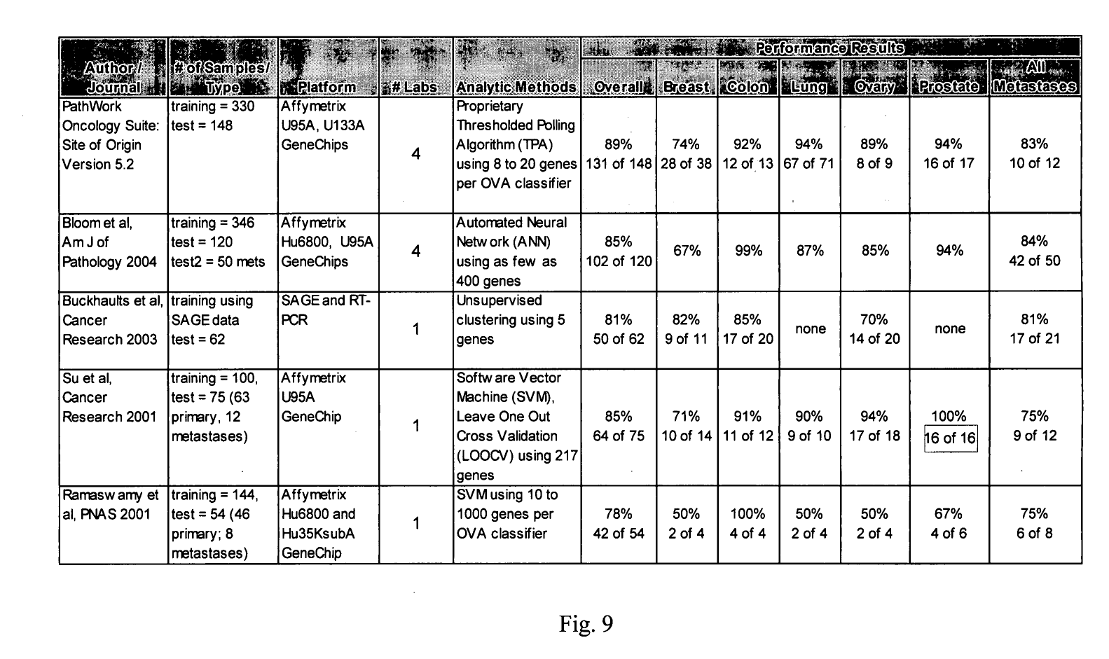

In alternative embodiments, candidate upper threshold p^{thres} and lower threshold n^{thres} values must satisfy the conditions that (i) p^{thres} and n^{thres} are points in a convex hull of the ROC curve, and (ii) p^{thres}-n^{thres} is greater than a predetermined value, such as 0.3, 0.5, etc. The convex hull of an ROC curve is the set of points in the plane is the ROC curve that are obtained if an elastic band was stretched around the outside of the points comprising the ROC curve and then snapped tight. For example, in ROC curve illustrated in FIG. 8, points 802 comprise the convex hull.

Table 5 represents a very limited data set. As such, it has a very limited convex set. However, in practice, the training data set partition is a larger data set. Because of the larger size of the training set partition, in practice, there will be more points that are part of the requisite convex set. For example, in some ROC curves there will be 3, 4, 5, 6, 7, 8, 9, 10 or more points in the desired convex set. The convex set represented in Table 8 is a more typical example of the set of points that belong to an acceptable convex set. In some instances, two points in the convex set will be very close in value. Therefore, in order in ensure that there is a sufficiently large indeterminate region (where the test votes “0” rather than “+1” or “−1”), the requirement that p^{thres}−n^{thres} is greater than a predetermined value, such as 0.3, 0.5, etc., is imposed.

In some embodiments, the actual candidate thresholds (p^{thres} and n^{thres}) are not the cutoff levels corresponding to points in the desired convex set. For example, in the case where ratio values are used to form the cutoff levels as in the case of Table 7 and FIG. 7, the ratio values are not used as candidate threshold values. Rather, what is used is the mean between (i) the cutoff level used to generate a given point in the convex set and (ii) the cutoff level used to generate the point immediately to the left of the given point in the convex set. For example, consider point 706 in FIG. 7. The ratio value 202 (from Table 5) was used as the cutoff level to generate point 706. The point in the ROC curve immediately to the left of point 706 in FIG. 7 is point 708. The ratio value 374 (from Table 5) was used as the cutoff level to generate point 708. Thus, when point 706 is considered as a candidate threshold, the ratio ((202+374)/2) or 288 is used as the candidate threshold. In such embodiments, the requirement that p^{thres}-n^{thres} is greater than a predetermined value means that p^{thres} is greater than n^{thres} and that the mean values generated by considering the points to the left of the p^{thres}, n^{thres} pair must deviate by more than a predetermined amount, such as 0.3. In some embodiments, the cutoff level used to generate the points in the desired convex set, as opposed to mean values, are used to generate candidate p^{thres}, n^{pairs}.

Table 8 illustrates hypothetical data that is obtained from an ROC curve for one test in a plurality of tests in the model under consideration. The table provides each possible pair of points in the ROC curve that satisfy the conditions specified above.

TABLE 8
Hypothetical candidate ROC data points and their corresponding
p^{thres}, n^{thres} values

| ROC data | | ROC data | |
|---|---|---|---|
| point for | Corresponding p^{thres} | point for | Corresponding |
| p^{thres} | threshold | n^{thres} | n^{thres} threshold |
| 9 | 30.5 | 7 | 20.2 |
| 7 | 20.2 | 4 | 6.0 |
| 4 | 6.0 | 2 | 3.7 |
| 9 | 30.5 | 4 | 6.0 |
| 9 | 30.5 | 2 | 3.7 |
| 7 | 20.2 | 2 | 3.7 |

As illustrated in Table 8, the desired convex set comprises data points 2, 4, 7, and 9. Thus, there are six possible candidate p^{thres}, n^{thres} values for the hypothetical candidate curve.

In preferred embodiments, candidate p^{thres}, n^{thres} values are determined for all or a portion of the tests in the model under consideration using the criteria described above. Then the model is tested against the training data set partition by exhaustively sampling all combinations of identified thresholds. In preferred embodiments, each such sampling comprises computing and scoring a goal function. The combination of thresholds that maximizes the goal function represent the desired threshold for use in the model. To illustrate, consider the case in which the model under consideration consists of tests A and B. Further suppose that there are two possible candidate p^{thres}, n^{thres} pairs for each test. That is, test A has a first candidate p^{thres}, n^{thres} pair denoted A1 and a second candidate p^{thres}, n^{thres} pair denoted A2. Likewise, test B has a first candidate p^{thres}, n^{thres} pair denoted B1 and a second candidate p^{thres}, n^{thres} pair denoted B2. This leads to four possible combinations to sample against the goal function in order to identify the best scoring combination. Namely, the four possible combinations are (A1, B1), (A1, B2), (A2, B1) and (A2, B2).

In a preferred embodiment, an ROC curve is generated for each combination of identified thresholds using the training data set partition. In the example described above, this means that a first ROC curve is generated using the (A1, B1) thresholds, a second ROC curve is generated using the (A1, B2) thresholds, and so forth. Table 9 illustrates the data that is used to form an ROC curve using the (A1, B1) thresholds.

TABLE 9
Values for a model for feature S using data from the training set

| Combined vote of each | |
|---|---|
| test in the model | Presence/Absence of Feature S |
| 2 | Y |
| 2 | Y |
| 1 | Y |
| 1 | Y |
| 0 | N |
| −1 | Y |
| −2 | N |
| −2 | N |

Each row in Table 9 corresponds to a different biological organism/specimen in the training data set partition. The left column represents the combined votes of test A and test B in the model being sampled. The thresholds used for the application of these tests to generate the data of Table 9 are the (A1, B1) thresholds. The biological organisms/specimens in Table 9 are ranked by the score in the left hand column. The right hand column details the presence or absence of feature S in the corresponding biological organisms/specimens of the training data set partition. Once an ROC curve has been computed for a set of thresholds to be evaluated, the point in the ROC curve (the 1-specificity, sensitivity coordinate) that separates the +1 and the 0 votes is determined. In one embodiment of the present invention, the goal function is 7*specificity+sensitivity, where the specificity and sensitivity values are taken from the point in the ROC curve that corresponds to the point that separates the +1 and the 0 votes. In the example illustrated in Table 9, this point in the ROC curve that separates the +1 and the 0 votes is between the fourth and the fifth rows of the table.

Each possible combination of thresholds is used to generate an ROC curve as described above. The sensitivity and specificity of the point that separates the +1 and the 0 votes is polled and used as the basis for a goal function. The threshold combination (e.g. A1, B1) that generates the highest goal function or near highest goal function is then selected as the thresholds used in the model.

Step 620.

In step 620, process control is returned to step 608 where another feature class S from the plurality of feature classes under investigation is selected. Then, steps 608 through 618 are repeated until a model has been constructed for each feature class S in the plurality of feature classes under investigation.

Step 622.

In step 622, the performance of each model constructed in preceding steps is tested against the test data set partition. Each test in a model contributes one vote for each specimen tested. For example, if there are eight tests in a model, a total of eight votes are made for each specimen considered by the model. In some embodiments, each test contributes a “+1” vote, a “0” vote, or a “−1” vote. The model tests positive for the feature S associated with the model if the summation of the votes of the model's test is a positive number. The model tests negative for the feature S associated with the model if the summation of the votes of the model's test is zero or negative.

The present invention provides a number of different test combination methods. The straight voting scheme in which each test in a model gives a “+1”, “−1” or “0” vote has been described. In some embodiments, each test is weighted by the distance the polled test is away from its positive and/or negative thresholds. For instance, in some embodiments, the more a polled test exceeds its positive threshold, the more weight the test is given. In some embodiments, each test is weighted by the degree of confidence in the test. For example, in some embodiments, a test is weighted by the area under the ROC curve (area 702 of FIG. 7) used to generate the test. In such embodiments, tests corresponding to ROC curves with greater area under the curve are assigned larger weights than tests corresponding to ROC curves with smaller areas under the curve. Such embodiments assume that the predictive power of a test corresponds to the area under the ROC curve, with larger areas indicating more predictive power and smaller areas indicating less predictive power. In some embodiments, each polled test is weighted by the slope of the ROC curve at the exact test point being polled. For example, consider the case in which a test is the characteristic of cellular constituent A divided by the characteristic of cellular constituent B. To poll the test, the characteristic of cellular constituent A and cellular constituent B in the organism or biological specimen to be sample is obtained and the ratio of the two characteristics (e.g., abundances) is computed. Then, the slope of the ROC curve associated with the test is determined at the point on the curve corresponding to the computed value of the ratio. This slope is then used to weight the vote of the test. In preferred embodiments, slopes that approach the horizontal cause more weight to be assigned to a polled test and slopes that approach the vertical cause less weight to be assigned to a polled test.

Optionally, the tests of a model are modified by repeating steps 616 and 618 in order to attempt to improve model results. When repeating step 616, alternative tests that poll different cellular constituents can be incorporated into the model and existing tests can be deleted from the model. When a model has been finalized, it can optionally be tested against the validation data set partition for final validation/assessment of the model. However, once a model is tested against the validation data set partition, it is no longer modified.

### 5.10 Additional Embodiments

The section is directed to some specific embodiments of the present invention.

1. A method for constructing a classifier that classifies a biological specimen, comprising:
(A) calculating a plurality of test ratios for a biological sample class S, wherein each ratio in the plurality of test ratios comprises:
a numerator that is determined by an abundance of a first cellular constituent from a biological specimen, wherein the first cellular constituent is up-regulated or down-regulated in the biological sample class S relative to another biological sample class; and
a denominator that is determined by an abundance of a second cellular constituent, wherein the abundance of the second cellular constituent is measured from the same biological specimen used to measure the abundance of the first cellular constituent; and wherein
the pair defined by said first cellular constituent and said second cellular constituent differs for each test ratio in said plurality of test ratios, and
the biological sample class S and at least one other biological sample class is represented by the plurality of test ratios and a plurality of biological specimens is represented by the plurality of test ratios; and
(B) selecting a set of cellular constituent pairs for the biological sample class S, thereby constructing said classifier, such that a given cellular constituent pair in the set of cellular constituent pairs forms a ratio r that is represented in said plurality of ratios and that has a true minimum that is greater than a false maximum, and
the true minimum for the given ratio r is a first lower threshold percentile in a distribution of a first subset of the plurality of test ratios calculated in step (A); wherein cellular constituent abundance data used to calculate each test ratio in the first subset of test ratios is from biological specimens that are members of the biological sample class S, and
the false maximum for the given ratio r is a first upper threshold percentile in a distribution of a second subset of the plurality of test ratios calculated in step (A); wherein cellular constituent abundance data used to calculate each test ratio in the second subset of test ratios is from biological specimens that are not members of the biological sample class S; and
wherein the numerator of each ratio in the first and second subsets of test ratios is determined by using abundance data of first cellular constituents having the same identity as the first cellular constituent that determines the numerator of the given ratio r, and the denominator of each ratio in the first and second subsets of test ratios is determined by using abundance data of second cellular constituents having the same identity as the second cellular constituent that determines the denominator of the given ratio r.

2. The method of claim 1, the method further comprising, prior to said calculating step (A), the step of:
obtaining, for each respective biological specimen B in the plurality of biological specimens, a set of cellular constituent abundance data comprising abundance data for a plurality of cellular constituents from the respective biological specimen B; wherein the cellular constituent abundance data obtained from the plurality of biological specimens is used in the calculating step (A) to calculate the plurality of test ratios.

3. The method of claim 2, the method further comprising standardizing each set of cellular constituent abundance data obtained for each respective biological specimen B in the plurality of biological specimens prior to said calculating step (A).

4. The method of claim 3 wherein a set of cellular constituent abundance data obtained for a respective biological specimen B in the plurality of biological specimens is standardized by dividing all cellular constituent abundance values in the set of cellular constituent abundance data by the median cellular constituent abundance value of the set.

5. The method of claim 4 wherein said standardizing further comprises replacing a cellular constituent abundance value, having a value of zero or less in the set of cellular constituent abundance data, with a fixed value.

6. The method of claim 5 wherein said fixed value is determined by the median cellular constituent abundance value of the set of cellular constituent abundance data.

7. The method of claim 6 wherein said fixed value is between 0.001 and 0.5 of the median cellular constituent abundance value of the set of cellular constituent abundance data.

8. The method of claim 1 wherein, in step (A), the first cellular constituent is up-regulated in the biological sample class S relative to another biological sample class and the second cellular constituent is down-regulated in the biological sample class S relative to another biological sample class.

9. The method of claim 1 wherein, in step (A), the first cellular constituent is down-regulated in the biological sample class S relative to another biological sample class and the second cellular constituent is up-regulated in the biological sample class S relative to another biological sample class.

10. The method of claim 1 wherein, in step (A), the second cellular constituent is up-regulated in a biological sample class, other than the biological sample class S, relative to the biological sample class S.

11. The method of claim 1 wherein a cellular constituent that is used as a first cellular constituent or a second cellular constituent in at least one ratio in said plurality of ratios is a nucleic acid or a ribonucleic acid and an abundance of said cellular constituent is obtained by measuring a transcriptional state of all or a portion of said cellular constituent in all or a portion of said plurality of biological specimens.

12. The method of claim 11 wherein said first cellular constituent and said second cellular constituent are each independently mRNA, cRNA or cDNA.

13. The method of claim 1 wherein a cellular constituent that is used as a first cellular constituent or a second cellular constituent in at least one ratio in said plurality of ratios is a protein and the abundance of said cellular constituent is obtained by measuring a translational state of said cellular constituent in all or a portion of said plurality of biological specimens.

14. The method of claim 1 wherein an abundance of a cellular constituent in a numerator or a denominator of a ratio in said plurality of ratios is determined using isotope-coded affinity tagging followed by tandem mass spectrometry analysis.

15. The method of claim 1 wherein the abundance of a cellular constituent that is used as a numerator or a denominator in at least one ratio in said plurality of ratios is determined by measuring an activity or a post-translational modification of cellular constituent.

16. The method of claim 1 wherein, in step (A), said first cellular constituent is up-regulated and the second cellular constituent is down-regulated in the biological sample class S relative to another biological sample class and wherein
the plurality of test ratios comprises:
   A×B×N test ratios
where
A is the number of up-regulated cellular constituents in the biological sample class S;
B is the number of down-regulated cellular constituents in the biological sample class S; and
N is the number of biological specimens in said plurality of biological specimens.

17. The method of claim 1 wherein, in step (A), the first cellular constituent is down-regulated and the second cellular constituent is up-regulated in the biological sample class S relative to another biological sample class and wherein
the plurality of test ratios comprises:
   A×B×N test ratios
where
A is the number of down-regulated cellular constituents in the biological sample class S;
B is the number of up-regulated cellular constituents in the biological sample class S; and
N is the number of biological specimens in said plurality of biological specimens.

18. The method of claim 1 wherein, in step (A), the second cellular constituent is up-regulated in a biological sample class, other than the biological sample class S, relative to said biological sample class, and wherein
the plurality of test ratios comprises:
   A×D×N test ratios
where
A is the number of up-regulated cellular constituents in the biological sample class S;
D is the total number of up-regulated cellular constituents in the plurality of biological sample classes with the exception of the biological sample class S; and
N is the number of biological specimens in the plurality of biological specimens.

19. The method of claim 4 wherein the given ratio r has a true median that is greater than a lower allowed value and less than a higher allowed value, wherein the true median for the given ratio r is the median value of the first subset of test ratios.

20. The method of claim 4 wherein the given ratio r has a numerator that is greater than a lower allowed value.

21. The method of claim 4 wherein the true minimum for the given ratio r is greater than a threshold value.

22. The method of claim 4 wherein the log₁₀(true median/false median) for the given ratio r is greater than a threshold value where
the true median for the given ratio r is the median value of the first subset of test ratios; and
the false median for the given ratio r is the median value of the second subset of test ratios.

23. The method of claim 4 wherein the log₁₀(true median/false median) for the given ratio r is greater than the log₁₀(true median/false median) of any other ratio rᵢ in the plurality of test ratios calculated for the biological sample class S, where
the true median for a ratio rᵢ in the plurality of test ratios is the median of a distribution of a third subset of test ratios selected from the plurality of test ratios, where the cellular constituent abundance data used to calculate each ratio in the third subset is from biological specimens that are members of the biological sample class S,
the false median for said ratio rᵢ is the median of a distribution of a fourth subset of test ratios selected from the plurality of test ratios, where the cellular constituent abundance data used to calculate each ratio in the fourth subset is from biological specimens that are not members of the biological sample class S; and
wherein the numerator of each ratio in the third and fourth subsets is determined by the same cellular constituents that determine the numerator of the ratio rᵢ and the denominator of each ratio in the third and fourth subsets is determined by the same cellular constituents that determine the denominator of the ratio rᵢ.

24. The method of claim 4 wherein said set of cellular constituent pairs comprises between two and one thousand cellular constituent pairs and wherein the true minimum of each respective ratio rᵢ formed by a cellular constituent pair in the set of cellular constituent pairs is greater than the false maximum of the respective ratio rᵢ, where
the true minimum for a ratio rᵢ is a second lower threshold percentile in a distribution of a third subset of test ratios selected from the plurality of test ratios; wherein the cellular constituent abundance data used to calculate each test ratio in the third subset is from biological specimens that are members of the biological sample class S, and
the false maximum for the ratio rᵢ is a second upper threshold percentile in a distribution of a fourth subset of test ratios selected from the plurality of test ratios; wherein the cellular constituent abundance data used to calculate each test ratio in the fourth subset is from biological specimens that are not members of the biological sample class S; and
wherein the numerator of each ratio in the third and fourth subsets is determined by the same cellular constituents that determine the numerator of the ratio rᵢ and the denominator of each ratio in the third and fourth subsets is determined by the same cellular constituents that determine the denominator of the ratio rᵢ.

25. The method of claim 24 wherein set of cellular constituent pairs comprises between three and one hundred cellular constituent pairs.

26. The method of claim 4 wherein
the first lower threshold percentile is between the first and seventieth percentile of the distribution of the first subset of test ratios, and
the first upper threshold percentile is between the thirtieth and ninety-ninth percentile of the distribution of the second subset of test ratios.

27. The method of claim 24 wherein
the second lower threshold percentile is between the first and seventieth percentile of the distribution of the third subset, and
the second upper threshold percentile is between the thirtieth and ninety-ninth percentile of the distribution of the fourth subset.

28. The method of claim 1 wherein a different first cellular constituent is up-regulated in the biological sample class S when the abundance of the different first cellular constituent in biological specimens of the biological sample class is greater than the abundance of at least seventy percent of the cellular constituents in a plurality of biological specimens of the biological sample class for which cellular constituent abundance measurements have been made.

29. The method of claim 1 wherein a different first cellular constituent is down-regulated in the biological sample class S when the abundance of the different first cellular constituent in biological specimens of the biological sample class is less than the abundance of at least thirty percent of the cellular constituents in a plurality of biological specimens of the biological sample class for which cellular constituent abundance measurements have been made.

30. The method of claim 1 wherein a cellular constituent is represented in more than one cellular constituent pair in said set of cellular constituent pairs.

31. The method of claim 1 wherein each cellular constituent pair in said set of cellular constituent pairs includes at least one cellular constituent that is not represented in any other cellular constituent pair in said set of cellular constituent pairs.

32. A computer readable medium having computer-executable instructions for performing the steps of the method of claim 1.

33. A method of classifying a biological specimen into one of a plurality of biological sample classes, the method comprising:
(A) for each respective biological sample class in the plurality of biological sample classes, calculating a respective value for each respective ratio in a plurality of ratios for the biological sample class, wherein each ratio in the plurality of ratios is formed using a different cellular constituent pair in a set of cellular constituent pairs that is uniquely associated with the respective biological sample class, where each said respective value is calculated using cellular constituent abundance values, from the biological specimen, for the cellular constituent pair used to form the respective ratio corresponding to the respective value, wherein
the numerator of each ratio in the plurality of ratios for a respective biological sample class in the plurality of biological sample classes is determined by an abundance of a cellular constituent that is up-regulated or down-regulated in the respective biological sample class, relative to another biological sample class, and each ratio in the plurality of ratios has a true minimum and a false maximum; wherein
the true minimum for a given ratio r in the plurality of ratios for a respective biological sample class is a lower threshold percentile in a distribution of a first subset of test ratios; wherein the cellular constituent abundance data used to calculate each test ratio in the first subset of test ratios is from a plurality of biological specimens that are members of the respective biological sample class, and
the false maximum for the given ratio r in the plurality of ratios for the respective biological sample class is an upper threshold percentile in a distribution of a second subset of test ratios; wherein the cellular constituent abundance data used to calculate each test ratio in the second plurality of test ratios is from a plurality of biological specimens that are not members of the respective biological sample class; and
the numerator of each ratio in the first and second subset of test ratios is determined by the same cellular constituent that determines the numerator of the given ratio r, and the denominator of each ratio in the first and second subset of test ratios is determined by the same cellular constituent that determines the denominator of the given ratio r;
(B) for each respective biological sample class in the plurality of biological sample classes, for each respective ratio in the plurality of ratios associated with the respective biological sample class:
identifying the respective ratio as negative when a value of the ratio that was calculated in step (A) is below the true minimum for the ratio;
identifying the respective ratio as positive when the value of the ratio that was calculated in step (A) is above the false maximum for the ratio; and
identifying the respective ratio as indeterminate when the value of the ratio that was calculated in step (A) is above the true minimum and below the false maximum for the ratio; and
(C) for each respective biological sample class in the plurality of biological sample classes,
identifying the set of cellular constituent pairs associated with the respective biological sample class as positive when more ratios in the plurality of ratios corresponding to said set of cellular constituent pairs are identified as positive than are identified as negative in step (B), wherein,
when the set of cellular constituent pairs associated with only one biological sample class in the plurality of biological sample classes is identified as positive in step (C), the biological specimen is classified into the biological sample class associated with the set of cellular constituent pairs that was identified as positive.

34. The method of claim 33, the method further comprising, prior to said step (A), the step of:
obtaining a set of cellular constituent abundance data, wherein
   the set of cellular constituent abundance data includes abundance data for the cellular constituent that determines the numerator of the given ratio r in the plurality of ratios for a respective biological sample class in the plurality of biological sample classes; and
   the set of cellular constituent abundance data includes abundance data for the cellular constituent that determines the denominator of the given ratio r.

35. The method of claim 34, the method further comprising standardizing the set of cellular constituent abundance data.

36. The method of claim 35 wherein the standardizing the set of cellular constituent abundance data comprises dividing all cellular constituent abundance values in the set of cellular constituent abundance data by the median cellular constituent abundance value of the set.

37. The method of claim 36 wherein the standardizing further comprises replacing a cellular constituent abundance value, in the set of cellular constituent abundance data, that has a value of zero or less, with a fixed value.

38. The method of claim 37 wherein the fixed value is determined by the median cellular constituent abundance value of the set of cellular constituent abundance data.

39. The method of claim 37 wherein the fixed value is between 0.001 and 0.5 of the median cellular constituent abundance value of the set of cellular constituent abundance data.

40. The method of claim 34 wherein a cellular constituent having an abundance value in the set of cellular constituent abundance data is a nucleic acid or a ribonucleic acid and the abundance value of the cellular constituent is obtained by measuring a transcriptional state of all or a portion of the cellular constituent in a biological specimen.

41. The method of claim 40 wherein the cellular constituent is mRNA, cRNA or cDNA.

42. The method of claim 34 wherein a cellular constituent having an abundance value in the set of cellular constituent abundance data is a protein and the abundance of the cellular constituent is obtained by measuring a translational state of all or a portion of the cellular constituent in a biological specimen.

43. The method of claim 34 wherein an abundance of a cellular constituent represented in the set of cellular constituent abundance data is determined using isotope-coded affinity tagging followed by tandem mass spectrometry analysis.

44. The method of claim 34 wherein an abundance of a cellular constituent represented in the set of cellular constituent abundance data is determined by measuring an activity or a post-translational modification of the cellular constituent in a biological specimen.

45. The method of claim 34 wherein an abundance of a cellular constituent represented in the set of cellular constituent abundance data is determined by measuring an activity or a post-translational modification of the cellular constituent.

46. The method of claim 34 wherein a given ratio in the plurality of ratios for a biological sample class in the plurality of biological sample classes has a true median that is greater than a lower allowed value and less than a higher allowed value, wherein the true median for the given ratio is the median value of the first subset of test ratios of step (A).

47. The method of claim 34 wherein a given ratio in the plurality of ratios for a biological sample class in the plurality of biological sample classes has a numerator that is greater than a lower allowed value.

48. The method of claim 34 wherein the true minimum for a given ratio in the plurality of ratios for a biological sample class in the plurality of biological sample classes is greater than a threshold value.

49. The method of claim 48 wherein the true minimum for a given ratio in the plurality of ratios for a biological sample class in the plurality of biological sample classes is at least 1.2 times the false maximum.

50. The method of claim 34 wherein the log₁₀(true median/false median) for a given ratio in the plurality of ratios for a biological sample class in the plurality of biological sample classes is greater than a threshold value where
the true median for the given ratio is the median value of the first subset of test ratios; and
the false median for the given ratio is the median value of the second subset of test ratios.

51. The method of claim 33 wherein the plurality of ratios for a biological sample class in the plurality of biological sample classes comprises between two and one thousand ratios.

52. The method of claim 33 wherein the plurality of ratios for a biological sample class in the plurality of biological sample classes comprises between two and one hundred ratios.

53. The method of claim 33 wherein
the lower threshold percentile is between the first and seventieth percentile of the distribution of the first subset of test ratios, and
the upper threshold percentile is between the thirties and ninety-ninth percentile of the distribution of the second subset of test ratios.

54. The method of claim 33 wherein the cellular constituent is up-regulated in the respective biological sample class when the abundance of the cellular constituent in biological specimens of the biological sample class is greater than the abundance of at least seventy percent of the cellular constituents in biological specimens of the biological sample class for which cellular constituent abundance measurements have been made.

55. The method of claim 33 wherein the first cellular constituent is down-regulated in the respective biological sample class when the abundance of the cellular constituent in biological specimens of the biological sample class is less than the abundance of at least thirty percent of the cellular constituents in biological specimens of the biological sample class for which cellular constituent abundance measurements have been made.

56. A computer readable medium having computer-executable instructions for performing the steps of the method of claim 33.

57. A method of classifying a biological specimen into a biological sample class, the method comprising:
(A) calculating a respective value for each respective ratio in a plurality of ratios for the biological sample class, wherein each ratio in the plurality of ratios is formed using a different cellular constituent pair in a set of cellular constituent pairs for the biological sample class, where each said respective value is calculated using cellular constituent abundance values, from the biological specimen, for the cellular constituent pair used to form the respective ratio corresponding to the respective value, wherein
the numerator of each ratio in the plurality of ratios is determined by an abundance of a cellular constituent that is up-regulated or down-regulated in the biological sample class relative to another biological sample class and each ratio in the plurality of ratios has a true minimum and a false maximum; wherein
the true minimum for a given ratio r in the plurality of ratios is a lower threshold percentile in a distribution of a first subset of test ratios; wherein the cellular constituent abundance data used to calculate each test ratio in the first subset of test ratios is from a plurality of biological specimens that are members of the biological sample class, and
the false maximum for the given ratio r in the plurality of ratios is an upper threshold percentile in a distribution of a second subset of test ratios; wherein the cellular constituent abundance data used to calculate each test ratio in the second plurality of test ratios is from a plurality of biological specimens that are not members of the biological sample class; and
the numerator of each ratio in the first and second subset of test ratios is determined by the same cellular constituent that determines the numerator of the given ratio r and the denominator of each ratio in the first and second subset of test ratios is determined by the same cellular constituent that determines the denominator of the given ratio r;
(B) for each respective ratio in the plurality of ratios:
identifying the respective ratio as negative when a value of the ratio that was calculated in step (A) is below true minimum for the ratio;
identifying the respective ratio as positive when the value of the ratio that was calculated in step (A) is above the false maximum for the ratio; and
identifying the respective ratio as indeterminate when the value of the ratio that was calculated in step (A) is above the true minimum and below the false maximum for the ratio; and
(C) classifying the biological specimen into the biological sample class when more ratios in the plurality of ratios corresponding to the set of cellular constituent pairs for the biological sample class are identified as positive than are identified as negative in step (B).

58. The method of claim 57, the method further comprising, prior to said step (A), the step of:
obtaining a set of cellular constituent abundance data, wherein
   the set of cellular constituent abundance data includes abundance data for the cellular constituent that determines the numerator of the given ratio r in the plurality of ratios; and
   the set of cellular constituent abundance data includes abundance data for the cellular constituent that determines the denominator of the given ratio r.

59. The method of claim 58, the method further comprising standardizing the set of cellular constituent abundance data.

60. The method of claim 57 wherein the standardizing the set of cellular constituent abundance data comprises dividing all cellular constituent abundance values in the set by the median cellular constituent abundance value of the set.

61. The method of claim 59 wherein the standardizing further comprises replacing a cellular constituent abundance value, in the set of cellular constituent abundance data, that has a value of zero or less, with a fixed value.

62. The method of claim 58 wherein a cellular constituent having an abundance value in the set of cellular constituent abundance data is a nucleic acid or a ribonucleic acid and the abundance value of the cellular constituent is obtained by measuring a transcriptional state of all or a portion of the cellular constituent in a biological specimen.

63. The method of claim 62 wherein the cellular constituent is mRNA, cRNA, or cDNA.

64. A computer readable medium having computer-executable instructions for performing the steps of the method of claim 57.

65. A computer program product for use in conjunction with a computer system, the computer program product comprising a computer readable storage medium and a computer program mechanism embedded therein, the computer program mechanism for classifying a biological specimen into a biological sample class, the computer program mechanism comprising one or more models, each model in said one or more models comprising:
a ratio data structure for the biological sample class, wherein the ratio data structure comprises between two and one thousand different ratios and wherein:
(i) a given ratio in the ratio data structure has a numerator that is determined by an abundance of a first cellular constituent in the biological specimen and a denominator that is determined by an abundance of a second cellular constituent in the biological specimen, and
(ii) a true minimum and a false maximum for the given ratio, wherein
the true minimum for the given ratio is a lower threshold percentile in a distribution of a first subset of test ratios;
the false maximum for the given ratio is an upper threshold percentile in a distribution of a second subset of test ratios;
a numerator of a test ratio in the first subset of test ratios is determined by an abundance of the first cellular constituent in any biological specimen of the biological sample class;
a denominator of a test ratio in the second subset of test ratios is determined by an abundance of the second cellular constituent in a biological specimen of the biological sample class;
a numerator of a test ratio in the second subset of test ratios is determined by an abundance of the first cellular constituent in a biological specimen not of the biological sample class; and
a denominator of a test ratio in the second subset of test ratios is determined by an abundance of the second cellular constituent in biological specimens not of the biological sample class.

66. The computer program product of claim 65 wherein, for each respective ratio in the ratio data structure having an associated true minimum and associated false maximum,
the respective ratio is identified as negative when a value of the ratio is below the true minimum associated with the ratio;
the respective ratio is identified as positive when a value of the ratio is above the false maximum associated with the ratio; and
the respective ratio is identified as indeterminate when the value of the ratio is above the true minimum and below the false maximum for the ratio; wherein
   the biological specimen is classified into the biological sample class when more ratios in the ratio data structure are identified as positive than are identified as negative.

67. The computer program product of claim 65 wherein, for each respective ratio in the ratio data structure having an associated true minimum and associated false maximum,
the respective ratio is identified as negative when a value of the ratio is above the true minimum associated with the ratio;
the respective ratio is identified as positive when a value of the ratio is below the false maximum associated with the ratio; and
the respective ratio is identified as indeterminate when the value of the ratio is below the true minimum and above the false maximum for the ratio; wherein the biological specimen is classified into the biological sample class when more ratios in the ratio data structure are identified as positive than are identified as negative.

68. The computer program product of claim 65 wherein the first cellular constituent is up-regulated or down-regulated in the biological sample class relative to another biological sample class.

69. The computer program product of claim 65 wherein the first cellular constituent is up-regulated in the biological sample class and the second cellular constituent is down-regulated in the biological sample class relative to another biological sample class.

70. The computer program product of claim 65 wherein the first cellular constituent is down-regulated in the biological sample class and the second cellular constituent is up-regulated in the biological sample class relative to another biological sample class.

71. The computer program product of claim 65, wherein the abundance of the first cellular constituent and the abundance of the second cellular constituent in the biological specimen is standardized against cellular constituent measurements for a plurality of cellular constituents from the biological specimen.

72. The computer program product of claim 71 wherein the standardizing comprises dividing the abundance of the first cellular constituent and the abundance of the second cellular constituent by the median cellular constituent abundance value of the cellular constituent measurements for the plurality of cellular constituents from the biological specimen.

73. The computer program product of claim 65, wherein the abundance of the first cellular constituent and the abundance of the second cellular constituent in the biological specimen that determine a test ratio in the first subset of test ratios or the second subset of test ratios is standardized against a plurality of cellular constituent measurements from the biological specimen from which the abundance of the first cellular constituent and the abundance of the second cellular constituent that determine the test ratio were obtained.

74. The computer program product of claim 73 wherein the standardizing comprises dividing the abundance of the first cellular constituent and the abundance of the second cellular constituent by the median cellular constituent abundance value of the cellular constituent measurements for the plurality of cellular constituents from the biological specimen.

75. The computer program product of claim 73 wherein the first cellular constituent is up-regulated in said biological sample class and said second cellular constituent is up-regulated in a biological sample class other than said biological sample class.

76. The computer program product of claim 73 wherein the first cellular constituent and the second cellular constituent are each a nucleic acid or a ribonucleic acid and the abundance of the first cellular constituent and the abundance of the second cellular constituent is obtained by measuring a transcriptional state of all or a portion of said first cellular constituent and said second cellular constituent.

77. The computer program product of claim 76 wherein the first cellular constituent and the second cellular constituent are each mRNA, cRNA or cDNA.

78. The computer program product of claim 65 wherein the first cellular constituent and the second cellular constituent are each proteins and the abundance of the first cellular constituent and the abundance of the second cellular constituent are obtained by measuring a translational state of all or a portion of said first cellular constituent and said second cellular constituent.

79. The computer program product of claim 65 wherein the abundance of the first cellular constituent and the second cellular constituent is determined by measuring an activity or a post-translational modification of the first cellular constituent and the second cellular constituent.

80. The computer program product of claim 71 wherein the given ratio has a true median that is greater than a lower allowed value and less than a higher allowed value, wherein the true median for the given ratio is the median value of the first subset of test ratios.

81. The computer program product of claim 71 wherein the log₁₀ (true median/false median) for the given ratio is greater than a threshold value where
the true median for the given ratio is the median value of the first subset of test ratios; and
the false median for the given ratio is the median value of the second subset of test ratios.

82. The computer program product of claim 71 wherein the true minimum of each respective ratio in the ratio data structure is greater than the false maximum of the respective ratio.

83. The computer program product of claim 65 wherein
the lower threshold percentile is between the tenth and thirtieth percentile of the distribution of the first subset of ratios; and
the upper threshold percentile is between the seventieth and ninety-fifth percentile of the distribution of the second subset of test ratios.

84. The computer program product of claim 65 wherein an abundance of the first cellular constituent is in biological specimens of the biological sample class is greater than the abundance of at least seventy percent of a plurality of cellular constituents in biological specimens of the biological sample class.

85. The computer program product of claim 65 wherein an abundance of the first cellular constituent in biological specimens of the biological sample class is less than the abundance of at least thirty percent of a plurality of cellular constituents in biological specimens of the biological sample class.

86. A computer program product for use in conjunction with a computer system, the computer program product comprising a computer readable storage medium and a computer program mechanism embedded therein, the model creation application for constructing a classifier that classifies a biological specimen, the model creation application comprising:
(A) a ratio computation module for calculating a plurality of test ratios for a biological sample class S, wherein each ratio in the plurality of test ratios comprises:
a numerator that is determined by an abundance of a first cellular constituent from a biological specimen, wherein the different first cellular constituent is up-regulated or down-regulated in the biological sample class S relative to another biological sample class; and
a denominator that is determined by an abundance of a second cellular constituent, wherein the abundance of the different second cellular constituent is measured from the same biological specimen used to measure the abundance of the first cellular constituent; and wherein
the pair defined by said first cellular constituent and said second cellular constituent differs for each test ratio in said plurality of test ratios, and
the biological sample class S and at least one other biological sample class is represented by the plurality of test ratios and a plurality of biological specimens is represented by the plurality of test ratios; and
(B) a ratio selection module for selecting a set of cellular constituent pairs for the biological sample class S, thereby constructing said classifier, such that a given cellular constituent pair in the set of cellular constituent pairs forms a ratio r that is represented in said plurality of ratios and that has a true minimum that is greater than a false maximum, and,
the true minimum for the given ratio r is a first lower threshold percentile in a distribution of a first subset of the plurality of test ratios calculated by said ratio computation model; wherein cellular constituent abundance data used to calculate each test ratio in the first subset of test ratios is from biological specimens that are members of the biological sample class S, and
the false maximum for the given ratio r is a first upper threshold percentile in a distribution of a second subset of the plurality of test ratios calculated by said ratio computation model; wherein cellular constituent abundance data used to calculate each test ratio in the second subset of test ratios is from biological specimens that are not members of the biological sample class S; and
wherein the numerator of each ratio in the first and second subsets of test ratios is determined by using abundance data of first cellular constituents having the same identity as the first cellular constituent that determines the numerator of the given ratio r, and the denominator of each ratio in the first and second subsets of test ratios is determined by using abundance data of second cellular constituents having the same identity as the second cellular constituent that determines the denominator of the given ratio r.

87. The computer program product of claim 86, the model creation application further comprising a standardization module for standardizing the abundance of the first cellular constituent and the abundance of the second cellular constituent from the biological specimen.

88. The computer program product of claim 87 wherein the standardizing comprises dividing the abundance of the first cellular constituent and the abundance of the second cellular constituent by the median cellular constituent abundance value of a plurality of cellular constituent abundance values from the biological specimen.

89. A computer system for constructing a classifier that classifies a biological specimen into one of a plurality of biological sample classes, the computer system comprising:
a central processing unit;
a memory, coupled to the central processing unit, the memory storing a model creation application; wherein the model creation application comprises:
a model creation application, the model creation application comprising:
(A) a ratio computation module for calculating a plurality of test ratios for a biological sample class S, wherein each ratio in the plurality of test ratios comprises:
a numerator that is determined by an abundance of a different first cellular constituent from a biological specimen, wherein the different first cellular constituent is up-regulated or down-regulated in the biological sample class S relative to another biological sample class; and
a denominator that is determined by an abundance of a different second cellular constituent, wherein the abundance of the different second cellular constituent is measured from the same biological specimen used to measure the abundance of the first cellular constituent; and wherein
the biological sample class S and at least one other biological sample class is represented by the plurality of test ratios and a plurality of biological specimens is represented by the plurality of test ratios; and
(B) a ratio selection module for selecting a set of cellular constituent pairs for the biological sample class S, thereby constructing said classifier, such that a given cellular constituent pair in the set of cellular constituent pairs forms a ratio r that is represented in said plurality of ratios and that has a true minimum that is greater than a false maximum, and,
the true minimum for the given ratio r is a first lower threshold percentile in a distribution of a first subset of the plurality of test ratios calculated by said ratio computation model; wherein the cellular constituent abundance data used to calculate each test ratio in the first subset of test ratios is from biological specimens that are members of the biological sample class S, and
the false maximum for the given ratio r is a first upper threshold percentile in a distribution of a second subset of test ratios selected from the plurality of test ratios; wherein the cellular constituent abundance data used to calculate each test ratio in the second subset of test ratios is from biological specimens that are not members of the biological sample class S; and
wherein the numerator of each ratio in the first and second subsets of test ratios is determined by cellular constituents having the same identity as the cellular constituent that determines the numerator of the given ratio r and the denominator of each ratio in the first and second subsets of test ratios is determined by cellular constituents having the same identity as the cellular constituent that determines the denominator of the given ratio r.

90. The computer system of claim 89, the model creation application further comprising a standardization module for standardizing the abundance of the first cellular constituent and the abundance of the second cellular constituent from the biological specimen.

91. The computer system of claim 90 wherein the standardizing comprises dividing the abundance of the first cellular constituent and the abundance of the second cellular constituent by the median cellular constituent abundance value of a plurality of cellular constituent abundance values from the biological specimen.

92. The computer system of claim 91 wherein said standardizing further comprises replacing a cellular constituent abundance value, in the plurality of cellular constituent abundance values, having a value of zero or less, with a fixed value.

93. The computer system of claim 89 wherein the first cellular constituent is up-regulated and the second cellular constituent is down-regulated in the biological sample class S relative to another biological sample class.

94. The computer system of claim 89 wherein the first cellular constituent is down-regulated and the second cellular constituent is up-regulated in the biological sample class S relative to another biological sample class.

95. The computer system of claim 89 wherein the second cellular constituent is up-regulated in a biological sample class other than the biological sample class S relative to another biological sample class.

96. The computer system of claim 89 wherein the first cellular constituent and the second cellular constituent are each a nucleic acid or a ribonucleic acid.

97. The computer system of claim 96 wherein the first cellular constituent and the second cellular constituent are each mRNA, cRNA or cDNA.

98. The computer system of claim 89 wherein the first cellular constituent and the second cellular constituent are each proteins.

99. The computer system of claim 89 wherein the abundance of the first cellular constituent and the abundance of second cellular constituent is determined by measuring an activity or a post-translational modification of the first cellular constituent and the second cellular constituent.

100. The computer system of claim 89 wherein the first cellular constituent is up-regulated and the second cellular constituent is down-regulated in the biological sample class S relative to another biological sample class and wherein
the plurality of test ratios for the biological sample class S comprises:
   A×B×N test ratios
where
A is the number of up-regulated cellular constituents in the biological sample class S;
B is the number of down-regulated cellular constituents in the biological sample class S; and
C is the number of biological specimens used in the computation of the plurality of test ratios by said ratio computation module.

101. The computer system of claim 89 wherein the first cellular constituent is down-regulated and the second cellular constituent is up-regulated in the biological sample class S relative to another biological sample class and wherein
the plurality of test ratios for the biological sample class S comprises:
   A×B×N test ratios
where
A is the number of down-regulated cellular constituents in the biological sample class S;
B is the number of up-regulated cellular constituents in the biological sample class S; and
N is the number of biological specimens used in the computation of the plurality of test ratios by said ratio computation module.

102. The computer system of claim 89 wherein the second cellular constituent is up-regulated in a biological sample class, other than the biological sample class S, relative to the biological sample class and wherein the plurality of test ratios for the biological sample class S comprises:
A×D×N test ratios
where
A is the number of up-regulated cellular constituents in the biological sample class S;
D is the total number of up-regulated cellular constituents in the plurality of biological sample classes with the exception of the biological sample class S; and
N is the number of biological specimens used in the computation of the plurality of test ratios by said ratio computation module.

103. The computer system of claim 89 wherein the given ratio r has a true median that is greater than a lower allowed value and less than a higher allowed value, wherein the true median for the given ratio r is the median value of the first subset of test ratios selected from the plurality of test ratios calculated by said ratio computation module for the biological sample class S that the given ratio r represents.

104. The computer system of claim 89 wherein the log₁₀ (true median/false median) for the given ratio r is greater than a threshold value where
the true median for the given ratio r is the median value of the first subset of test ratios; and
the false median for the given ratio r is the median value of the second subset of test ratios.

105. The computer system of claim 89 wherein the log₁₀ (true median/false median) for the given ratio r is greater than the log₁₀ (true median/false median) of any other ratio rᵢ in the plurality of test ratios calculated for the biological sample class S, where
the true median for a ratio rᵢ in the plurality of test ratios is the median of a distribution of a third subset of test ratios selected from the plurality of test ratios, where the cellular constituent abundance data used to calculate each ratio in the third subset is from biological specimens that are members of the biological sample class S,
the false median for said ratio rᵢ is the median of a distribution of a fourth subset of test ratios selected from the plurality of test ratios, where the cellular constituent abundance data used to calculate each ratio in the fourth subset is from biological specimens that are not members of the biological sample class S; and
wherein the numerator of each ratio in the third and fourth subsets is determined by the same cellular constituents that determine the numerator of the ratio rᵢ and the denominator of each ratio in the third and fourth subsets is determined by the same cellular constituents that determine the denominator of the ratio rᵢ.

106. The computer system of claim 89 wherein the set of cellular constituent pairs comprises between two and one thousand cellular constituent pairs and wherein the true minimum of each respective ratio rᵢ corresponding to a cellular constituent pair in the set of cellular constituent pairs is greater than the false maximum of the respective ratio rᵢ, where
the true minimum for a ratio rᵢ is a second lower threshold percentile in a distribution of a third subset of test ratios selected from the plurality of test ratios; wherein the cellular constituent abundance data used to calculate each test ratio in the third subset is from biological specimens that are members of the biological sample class S, and
the false maximum for the ratio rᵢ is a second upper threshold percentile in a distribution of a fourth subset of test ratios selected from the plurality of test ratios; wherein the cellular constituent abundance data used to calculate each test ratio in the fourth subset is from biological specimens that are not members of the biological sample class S; and
wherein the numerator of each ratio in the third and fourth subsets is determined by the same cellular constituents that determine the numerator of the ratio rᵢ and the denominator of each ratio in the third and fourth subsets is determined by the same cellular constituents that determine the denominator of the ratio rᵢ.

107. The computer system of claim 89 wherein
the first lower threshold percentile is between the first and seventieth percentile of the distribution of the first subset of test ratios, and
the first upper threshold percentile is between the thirtieth and ninety-ninth percentile of the distribution of the second subset of test ratios.

108. The computer system of claim 89 wherein the first cellular constituent is up-regulated in the biological sample class S when the abundance of the first cellular constituent in biological specimens of the biological sample class is greater than the abundance of at least seventy percent of a plurality of cellular constituents in biological specimens of the biological sample class S.

109. The computer system of claim 89 wherein the first cellular constituent is down-regulated in the biological sample class S when the abundance of the first cellular constituent in biological specimens of the biological sample class is less than the abundance of at least thirty percent of a plurality of cellular constituents in biological specimens of the biological sample class S.

110. A computer program product for use in conjunction with a computer system, the computer program product comprising a computer readable storage medium and a model testing application embedded therein, the model testing application for classifying a biological specimen into one of a plurality of biological sample classes, the model testing application comprising:
(A) for each respective biological sample class in the plurality of biological sample classes, instructions for calculating a respective value for each respective ratio in a plurality of ratios for the biological sample class, wherein each ratio in the plurality of ratios is formed using a different cellular constituent pair in a set of cellular constituent pairs that distinguishes the respective biological sample class, where each said respective value is calculated using cellular constituent abundance values, from the biological specimen, for the cellular constituent pair used to form the respective ratio corresponding to the respective value, wherein
the numerator of each ratio in the plurality of ratios for a respective biological sample class in the plurality of biological sample classes is determined by an abundance of a cellular constituent that is up-regulated or down-regulated in the respective biological sample class relative to another biological sample class and each ratio in the plurality of ratios has a true minimum and a false maximum; wherein
the true minimum for a given ratio r in the plurality of ratios for a respective biological sample class is a lower threshold percentile in a distribution of a first subset of test ratios; wherein the cellular constituent abundance data used to calculate each test ratio in the first subset of test ratios is from a plurality of biological specimens that are members of the respective biological sample class, and
the false maximum for the given ratio r in the plurality of ratios for the respective biological sample class is an upper threshold percentile in a distribution of a second subset of test ratios; wherein the cellular constituent abundance data used to calculate each test ratio in the second plurality of test ratios is from a plurality of biological specimens that are not members of the respective biological sample class; and
the numerator of each ratio in the first and second subset of test ratios is determined by the same cellular constituent that determines the numerator of the given ratio r and the denominator of each ratio in the first and second subset of test ratios is determined by the same cellular constituent that determines the denominator of the given ratio r;
(B) for each respective biological sample class in the plurality of biological sample classes, for each respective ratio in the plurality of ratios associated with the respective biological sample class:
instructions for identifying the respective ratio as negative when a value of the ratio that was calculated by said instructions for calculating (A) is below the true minimum for the ratio;
identifying the respective ratio as positive when the value of the ratio that was calculated by said instructions for calculating (A) is above the false maximum for the ratio; and
identifying the respective ratio as indeterminate when the value of the ratio that was calculated by said instructions for calculating (A) is above the true minimum and below the false maximum for the ratio; and
(C) for each respective biological sample class in the plurality of biological sample classes,
instructions for identifying the set of cellular constituent pairs associated with the respective biological sample class as positive when more ratios in the plurality of ratios corresponding to said set of cellular constituent pairs are identified as positive than are identified as negative, wherein,
when the set of cellular constituent pairs associated with only one biological sample class in the plurality of biological sample classes is identified as positive, the biological specimen is classified into the biological sample class associated with the set of cellular constituent pairs that was identified as positive.

111. A computer system for classifying a biological specimen into one of a plurality of biological sample classes, wherein each biological sample class is associated with a different set of cellular constituent pairs, the computer system comprising:
a central processing unit;
a memory, coupled to the central processing unit, the memory storing a model testing application; wherein the model testing application comprises:
(A) for each respective biological sample class in the plurality of biological sample classes, instructions for calculating a respective value for each respective ratio in a plurality of ratios for the biological sample class, wherein each ratio in the plurality of ratios is formed using a different cellular constituent pair in a set of cellular constituent pairs that distinguishes the respective biological sample class, where each said respective value is calculated using cellular constituent abundance values, from the biological specimen, for the cellular constituent pair used to form the respective ratio corresponding to the respective value, wherein
the numerator of each ratio in the plurality of ratios for a respective biological sample class in the plurality of biological sample classes is determined by an abundance of a cellular constituent that is up-regulated or down-regulated in the respective biological sample class relative to another biological sample class and each ratio in the plurality of ratios has a true minimum and a false maximum; wherein
the true minimum for a given ratio r in the plurality of ratios for a respective biological sample class is a lower threshold percentile in a distribution of a first subset of test ratios; wherein the cellular constituent abundance data used to calculate each test ratio in the first subset of test ratios is from a plurality of biological specimens that are members of the respective biological sample class, and
the false maximum for the given ratio r in the plurality of ratios for the respective biological sample class is an upper threshold percentile in a distribution of a second subset of test ratios; wherein the cellular constituent abundance data used to calculate each test ratio in the second plurality of test ratios is from a plurality of biological specimens that are not members of the respective biological sample class; and
the numerator of each ratio in the first and second subset of test ratios is determined by the same cellular constituent that determines the numerator of the given ratio r and the denominator of each ratio in the first and second subset of test ratios is determined by the same cellular constituent that determines the denominator of the given ratio r;
(B) for each respective biological sample class in the plurality of biological sample classes, for each respective ratio in the plurality of ratios associated with the respective biological sample class:
instructions for identifying the respective ratio as negative when a value of the ratio that was calculated by said instructions for calculating (A) is below the true minimum for the ratio;
identifying the respective ratio as positive when the value of the ratio that was calculated by said instructions for calculating (A) is above the false maximum for the ratio; and
identifying the respective ratio as indeterminate when the value of the ratio that was calculated by said instructions for calculating (A) is above the true minimum and below the false maximum for the ratio; and
(C) for each respective biological sample class in the plurality of biological sample classes,
instructions for identifying the set of cellular constituent pairs associated with the respective biological sample class as positive when more ratios in the plurality of ratios corresponding to said set of cellular constituent pairs are identified as positive than are identified as negative, wherein,
when the set of cellular constituent pairs associated with only one biological sample class in the plurality of biological sample classes is identified as positive, the biological specimen is classified into the biological sample class associated with the set of cellular constituent pairs that was identified as positive.

112. A computer program product for use in conjunction with a computer system, the computer program product comprising a computer readable storage medium and a model testing application embedded therein, the model testing application for classifying a biological specimen into a biological sample class, the model testing application comprising:
(A) instructions for calculating a respective value for each respective ratio in a plurality of ratios for the biological sample class, wherein each ratio in the plurality of ratios is formed using a different cellular constituent pair in a set of cellular constituent pairs for the biological sample class, where each said respective value is calculated using cellular constituent abundance values, from the biological specimen, for the cellular constituent pair used to form the respective ratio corresponding to the respective value, wherein
the numerator of each ratio in the plurality of ratios is determined by an abundance of a cellular constituent that is up-regulated or down-regulated in the biological sample class relative to another biological sample class and each ratio in the plurality of ratios has a true minimum and a false maximum; wherein
the true minimum for a given ratio r in the plurality of ratios is a lower threshold percentile in a distribution of a first subset of test ratios; wherein the cellular constituent abundance data used to calculate each test ratio in the first subset of test ratios is from a plurality of biological specimens that are members of the biological sample class, and
the false maximum for the given ratio r in the plurality of ratios is an upper threshold percentile in a distribution of a second subset of test ratios; wherein the cellular constituent abundance data used to calculate each test ratio in the second plurality of test ratios is from a plurality of biological specimens that are not members of the biological sample class; and
the numerator of each ratio in the first and second subset of test ratios is determined by the same cellular constituent that determines the numerator of the given ratio r and the denominator of each ratio in the first and second subset of test ratios is determined by the same cellular constituent that determines the denominator of the given ratio r;
(B) for each respective ratio in the plurality of ratios:
instructions for identifying the respective ratio as negative when a value of the ratio that was calculated by said instructions for calculating (A) is below true minimum for the ratio;
instructions for identifying the respective ratio as positive when the value of the ratio that was calculated by said instructions for calculating (A) is above the false maximum for the ratio; and
instructions for identifying the respective ratio as indeterminate when the value of the ratio that was calculated by said instructions for calculating (A) is above the true minimum and below the false maximum for the ratio; and
(C) instructions for classifying the biological specimen into the biological sample class when more ratios in the plurality of ratios corresponding to the set of cellular constituent pairs for the biological sample class are identified as positive than are identified as negative.

113. A computer system for classifying a biological specimen into a biological sample class, the computer system comprising:
a central processing unit;
a memory, coupled to the central processing unit, the memory storing a model testing application; wherein the model testing application comprises:
(A) instructions for calculating a respective value for each respective ratio in a plurality of ratios for the biological sample class, wherein each ratio in the plurality of ratios is formed using a different cellular constituent pair in a set of cellular constituent pairs for the biological sample class, where each said respective value is calculated using cellular constituent abundance values, from the biological specimen, for the cellular constituent pair used to form the respective ratio corresponding to the respective value, wherein
the numerator of each ratio in the plurality of ratios is determined by an abundance of a cellular constituent that is up-regulated or down-regulated in the biological sample class relative to another biological sample class and each ratio in the plurality of ratios has a true minimum and a false maximum; wherein
the true minimum for a given ratio r in the plurality of ratios is a lower threshold percentile in a distribution of a first subset of test ratios; wherein the cellular constituent abundance data used to calculate each test ratio in the first subset of test ratios is from a plurality of biological specimens that are members of the biological sample class, and
the false maximum for the given ratio r in the plurality of ratios is an upper threshold percentile in a distribution of a second subset of test ratios; wherein the cellular constituent abundance data used to calculate each test ratio in the second plurality of test ratios is from a plurality of biological specimens that are not members of the biological sample class; and
the numerator of each ratio in the first and second subset of test ratios is determined by the same cellular constituent that determines the numerator of the given ratio r and the denominator of each ratio in the first and second subset of test ratios is determined by the same cellular constituent that determines the denominator of the given ratio r;
(B) for each respective ratio in the plurality of ratios:
instructions for identifying the respective ratio as negative when a value of the ratio that was calculated by said instructions for calculating (A) is below true minimum for the ratio;
instructions for identifying the respective ratio as positive when the value of the ratio that was calculated by said instructions for calculating (A) is above the false maximum for the ratio; and
instructions for identifying the respective ratio as indeterminate when the value of the ratio that was calculated by said instructions for calculating (A) is above the true minimum and below the false maximum for the ratio; and
(C) instructions for classifying the biological specimen into the biological sample class when more ratios in the plurality of ratios corresponding to the set of cellular constituent pairs for the biological sample class are identified as positive than are identified as negative.

114. The method of claim 1 wherein each cellular constituent pair in said set of cellular constituent pairs has the same properties as said given cellular constituent pair in said set of cellular constituent pairs.

115. The method of claim 1 wherein a majority of cellular constituent pairs in said set of cellular constituent pairs has the same properties as said given cellular constituent pair in said set of cellular constituent pairs.

116. The method of claim 1 wherein at least two biological sample classes are represented in said plurality of test ratios.

117. The method of claim 1 wherein at least five biological sample classes are represented in said plurality of test ratios.

118. The method of claim 1 wherein between two and one hundred biological sample classes are represented in said plurality of test ratios.

119. The method of claim 1 wherein said plurality of biological specimens represents between two and four thousand biological specimens.

120. The method of claim 33 wherein said plurality of biological sample classes represents between two and one thousand biological sample classes.

### 6. EXAMPLES

The following examples are presented by way of illustration of the invention and are not limiting. The methods described in Sections 5.1 and 5.2 and illustrated in FIGS. 2 and 3 were used in the examples provided in Sections 6.1 and 6.2. The methods described in Section 5.9 were used in the example provided in Section 6.3

### 6.1 Alpha Validation—Cancer of Unknown Primary

In this example, the methods described in Section 5.1 and illustrated in FIG. 2 were applied to data derived from Su et al., 2001, Cancer Research 61, p. 7388 to develop classifiers for tumors from a variety of biological sample classes 56 (e.g., prostate, bladder/ureter, breast, colorectal). Therefore, a set 72 was created for each of these tumor classes. Then, the ratios were tested to determine how well they classified the tumors in Su et al. into the appropriate biological sample class 52.

The study conducted by Su et al. used gene expression data to classify human carcinomas according to their primary origin. Classification was based on expression profiles that characterize each type of cancer. Samples from eleven different tissue types were included in the study. As described more fully below, the classifiers developed using the methods described in Section 5.1 and tested using the methods described in Section 5.2 classified 80 percent of the 174 samples in Su et al. with a sensitivity of 100 percent and specificity of 99.8 percent, where sensitivity and specificity are defined in step 310 of Section 5.2, above.

Step 202.

The samples used in the study came from cancerous tumors in the following tissues: breast (BR), bladder (BL), colorectal (CO), gastroesophagus (GA), kidney (KI), lung adenocarcinoma (LA), liver (LI), lung squamous cell carcinoma (LS), ovary (OV), pancreas (PA), and prostate (PR). The origin site of the tissue samples was known. RNA was extracted from tumors of each tumor class and hybridized onto oliognucleotide microarrays (U95a GeneChip; Affymetrix Incorporated, Santa Clara, Calif.) as described in Su et al.

Step 204.

One data file that contained the gene expression data of the tissue was created for each sample. The expression value for each gene in each respective file was divided by the mean gene expression value of the respective file in order to standardize gene expression values.

Step 206.

The Su et al. study selected for genes that were up-regulated in each of the tumor classes. Therefore the model created in Su et al. did not include down-regulated candidates (206-No).

Steps 220, 222, 250, 252, and 254.

Steps 220, 222, 250, 252 and 254 were run on the data files as described in Section 5.1 and illustrated in FIG. 2. This resulted in 11 ratio sets 72, one for each tumor type. As described in step 252 of Section 5.1, each set 72 includes a predetermined number of cellular constituent pairs and each of these cellular constituent pairs uniquely defines a different ratio. In this example, each set 72 had between three to five cellular constituent pairs (3-5 ratios). Collectively the set of eleven sets 72 developed in this experiment are referred to as the Su-Hampton 2001 model and are set forth in Table 10 below.

TABLE 10
The Su-Hampton 2001 model developed using
the methods of the present invention

| | | Up-regulated gene(Affymetrix | Down-regulated gene |
|---|---|---|---|
| Version | Tissue name | accession ID) | (Affymetrix accession ID) |
| 3.1 | Bladder | 36555_at | 34194_at |
| 3.1 | Bladder | 37104_at | 40736_at |
| 3.1 | Bladder | 32527_at | 41721_at |
| 3.1 | Bladder | 1490_at | 33701_at |
| 3.1 | Bladder | 32448_at | 33693_at |
| 3.1 | Breast | 33878_at | 40635_at |
| 3.1 | Breast | 39945_at | 40763_at |
| 3.1 | Breast | 41348_at | 37351_at |
| 3.1 | Colorectal | 40736_at | 36878_f_at |
| 3.1 | Colorectal | 32972_at | 39654_at |
| 3.1 | Colorectal | 38739_at | 32558_at |
| 3.1 | Colorectal | 37423_at | 35226_at |
| 3.1 | Colorectal | 1582_at | 33377_at |
| 3.1 | Gastroesophagus | 31575_f_at | 35220_at |
| 3.1 | Gastroesophagus | 34851_at | 35226_at |
| 3.1 | Gastroesophagus | 31574_i_at | 37236_at |
| 3.1 | Gastroesophagus | 40451_at | 37148_at |
| 3.1 | Gastroesophagus | 34491_at | 40401_at |
| 3.1 | Kidney | 35220_at | 37554_at |
| 3.1 | Kidney | 34777_at | 39945_at |
| 3.1 | Kidney | 40954_at | 35226_at |
| 3.1 | Kidney | 39260_at | 32796_f_at |
| 3.1 | Kidney | 35243_at | 1582_at |
| 3.1 | Liver | 32771_at | 37402_at |
| 3.1 | Liver | 37202_at | 927_s_at |
| 3.1 | Liver | 33377_at | 36457_at |
| 3.1 | Liver | 261_s_at | 41111_at |
| 3.1 | Liver | 36342_r_at | 40635_at |
| 3.1 | Lung | 41165_g_at | 35778_at |
| 3.1 | Lung | 33274_f_at | 32972_at |
| 3.1 | Lung | 41827_f_at | 40046_r_at |
| 3.1 | Ovary | 37554_at | 1582_at |
| 3.1 | Ovary | 38749_at | 39654_at |
| 3.1 | Ovary | 35277_at | 37104_at |
| 3.1 | Ovary | 32625_at | 37351_at |
| 3.1 | Ovary | 1500_at | 31575_f_at |
| 3.1 | Pancreas | 41238_s_at | 35332_at |
| 3.1 | Pancreas | 39177_r_at | 41164_at |
| 3.1 | Pancreas | 39176_f_at | 35226_at |
| 3.1 | Pancreas | 36141_at | 33754_at |
| 3.1 | Pancreas | 34941_at | 34777_at |
| 3.1 | Prostate | 40794_at | 41827_f_at |
| 3.1 | Prostate | 41172_at | 34778_at |
| 3.1 | Prostate | 32200_at | 927_s_at |
| 3.1 | Prostate | 41468_at | 39649_at |
| 3.1 | Prostate | 41721_at | 38894_g_at |

Once the Su-Hampton 2001 model had been constructed, it was tested using the methods described in Section 5.2 and illustrated in FIG. 3. Steps 302 and 304 were skipped because the standardized expression data was already available for the tumor samples of Su et al.

Steps 306 and 308.

The measures of sensitivity and specificity are traditionally used for the purpose of summarizing the quality of tests, such as models 72. However, sensitivity and specificity are designed to compare binary tests that detect presence or absence of a given feature. Thus only two outcomes are possible for these tests: positive (the feature is present) or negative (the feature is absent). The following truth table represents the distribution of samples depending on whether the feature is present or not, and what the model predicts. There are four possible classifications of samples: True Positives (TP), False Positives (FP), False Negatives (FN), and True Negatives (TN).

| | | Truth | |
|---|---|---|---|
| | | Feature Present | Feature Absent |
| Prediction | Positive | True Positives | False Positives |
| | Negative | False Negatives | True Negatives |

Sensitivity is a measure of the ability of a test to correctly identify the Feature when the Feature is present. Thus:

$$Sensitivity = \frac{TP}{TP + FN}$$

Specificity is a measure of the ability of a test to avoid making incorrect detections. Note that, in the case of a binary test, this is equivalent to the ability to correctly detect the absence of the Feature when the Feature is absent. This is not so for multi-valued tests as will be examined below.

$$Specificity = \frac{TN}{FP + TN}$$

However, as described in step 306 of Section 5.2, the ratios tested in the present invention do not produce binary results. This is for two reasons. First, an indetermined outcome is possible even in the case of otherwise binary tests. This is especially useful in medical diagnosis when the cost of an erroneous diagnosis is much higher than that of a lack of diagnosis. Second, some suites of sets 72, such as Site of Cancer Origin Verification, have intrinsically multivalued outcomes. Therefore, the output of such a test is not a simple “Positive” or “Negative” but one of a larger number of possibilities. For example, the tissue of origin of the tumor in the case of the Site of Cancer Origin Verification. Therefore, traditional notions of sensitivity and specificity do not adequately characterize the inherently non-binary tests used in the present invention and thus a different approach is required to validate and compare PWI models both internally and externally.

A natural extension of Sensitivity and Specificity to the multivariate test is given by the fraction of correct classifications and that of incorrect classifications. The following table shows an example of the classification of samples that have exactly one of three possible features, and have been tested with a test that will yield a prediction of which feature is present or “undetermined” if the results of the test were inconclusive. In this case there are twelve possible classifications, which can be divided into three categories (i) correct, (ii) incorrect, and (iii) inconclusive. In the general case where there are n different features, the total number of classifications is n (n+1).

| | | Truth | | |
|---|---|---|---|---|
| | | Feat. 1 Present | Feat. 2 Present | Feat. 3 Present |
| Pre- | Feat. 1 | Correct (1) | Incorrect (1, 2) | Incorrect (1, 3) |
| dic- | Present | | | |
| tion | Feat. 2 | Incorrect (2, 1) | Correct (2) | Incorrect (2, 3) |
| | Present | | | |
| | Feat. 3 | Incorrect (3, 1) | Incorrect (3, 2) | Correct (3) |
| | Present | | | |
| | Unde- | Inconclusive (1) | Inconclusive (2) | Inconclusive (3) |
| | ter- | | | |
| | mined | | | |

The total number of samples can be computed by adding all possible classifications:

$$total = {\sum }_{i = 1}^{n} Correct ( i ) + {\sum }_{i = 1}^{n} {\sum }_{{j = 1}_{j \neq i}}^{n} Incorrect ( i , j ) + {\sum }_{i = 1}^{n} Indeterminate ( i )$$

Fraction of samples correctly identified:

$$Correct = \frac{{\sum }_{i = 1}^{n} Correct ( i )}{total} \qquad ( I )$$

Fraction of samples incorrectly identified:

$$Incorrect = \frac{{\sum }_{i = 1}^{n} {\sum }_{{j = 1}_{j \neq i}}^{n} Incorrect ( i , j )}{total} \qquad ( II )$$

Fraction of samples for which the test offered inconclusive results and were not identified:

$$Indeterminate = \frac{{\sum }_{i = 1}^{n} Indeterminate ( i )}{total} \qquad ( III )$$

The eleven tests Su-Hampton 2001 were run for each biological specimen 58 from Su et al. Each test consisted of calculating each ratio defined by a given set 72 and determining whether the ratio was correct, incorrect, or indeterminate as respectively defined by equations (I), (II) and (III), above. The characterization of each of the eleven sets 72 was reviewed to determine whether a conclusion could be drawn about the particular sample's origin site.

Step 310.

Table 11 shows the results of the classification system used in Su et al. to classify each of the tumors (biological specimens 58) in the reference. As seen in Table 11, Su et al. was able to classify the tumors with an overall percent specificity of 1740/1747 or 99 percent and an overall percent sensitivity was 167/174 or 96 percent. There were seven samples that were incorrectly classified. As will be shown in subsequent tables below (see Table 14 in particular), the Su-Hampton 2001 model produced better results than those achieved by Su et al. using the same data.

TABLE 11
Summary of percent specificity and percent sensitivity
achieved by Su et al.

| | | Percent | Percent | Percent |
|---|---|---|---|---|
| | Origin Site | Specificity | Sensitivity | Indeterminate |
| BL | Bladder | 99 | 100 | 0 |
| BR | Breast | 99 | 100 | 0 |
| CO | Colorectal | 100 | 100 | 0 |
| GA | Gastroesophagus | 100 | 85 | 0 |
| KI | Kidney | 100 | 100 | 0 |
| LA | Lung Adenocarcinoma | 98 | 93 | 0 |
| LI | Liver | 100 | 71 | 0 |
| LS | Lung Squamous Cell | 100 | 93 | 0 |
| | Carcinoma | | | |
| OV | Ovary | 100 | 96 | 0 |
| PA | Pancreas | 100 | 100 | 0 |
| PR | Prostate | 100 | 100 | 0 |
| | Overall | 99 | 96 | 0 |

In Table 12, the predicted tissue type for each sample in Su et al. is described.

These predictions were made using the sets 72 calculated above (i.e., the Su-Hampton 2001 model). In Table 12, a “1” in a tissue type column indicates a positive result for that tissue type, “?” indicates an indeterminate result, and a “.” indicates a negative result. To the right of the eleven columns representing the eleven possible tissue types are columns representing the final classification of each sample. These final classifications are correct (COR), incorrect (INCOR), or indefinite (IND). Also reported is total (TOT), percent correct (% COR), percent incorrect (% INCOR), and percent indeterminate (% IND).

TABLE 12-1
Predicted tissue type for each bladder tumor sample in Su et al.

| SAMPLE (BL) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Bladder_BL10T | 1 | . | . | . | . | . | . | . | . | . | 1 | . | . | . | . | . | . |
| Bladder-BL16T | 1 | . | . | . | . | . | . | . | . | . | 1 | . | . | . | . | . | . |
| Bladder-BL18T | 1 | . | . | . | . | . | . | . | . | . | 1 | . | . | . | . | . | . |
| Bladder-BL19T | ? | . | . | . | . | . | . | . | . | . | . | . | 1 | . | . | . | . |
| Bladder-BL1T | 1 | . | . | . | . | . | . | . | . | . | 1 | . | . | . | . | . | . |
| Bladder-BL2T | 1 | . | . | . | . | . | 1 | . | . | . | . | . | 1 | . | . | . | . |
| Bladder-BL7T | 1 | . | . | . | . | . | . | . | . | . | 1 | . | . | . | . | . | . |
| Bladder-BL9T | 1 | . | . | . | . | . | . | . | . | . | 1 | . | . | . | . | . | . |

| | | | | | | | |
|---|---|---|---|---|---|---|---|
| SUMMARY | 6 | 0 | 2 | 8 | 75 | 0 | 25 |

TABLE 12-2
Predicted tissue type for each breast tumor sample in Su et al.

| SAMPLE (BR) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Breast-BR10T | . | 1 | . | . | . | . | . | . | . | . | 1 | . | . | . | . | . | . |
| Breast-BR14T | . | 1 | . | . | . | . | . | . | . | . | 1 | . | . | . | . | . | . |
| Breast-BR15T | . | 1 | . | . | . | . | . | . | . | . | 1 | . | . | . | . | . | . |
| Breast-BR16T | . | 1 | . | . | . | . | . | . | . | . | 1 | . | . | . | . | . | . |
| Breast-BR17T | . | 1 | . | . | . | . | . | . | . | . | 1 | . | . | . | . | . | . |
| Breast-BR20T | . | 1 | . | . | . | . | . | . | . | . | 1 | . | . | . | . | . | . |
| Breast-BR21T | . | 1 | . | ? | . | . | . | . | . | . | 1 | . | . | . | . | . | . |
| Breast-BR24T | . | 1 | . | 1 | . | . | . | . | . | . | . | . | 1 | . | . | . | . |
| Breast-BR29T | . | 1 | . | 1 | . | . | . | . | . | . | . | . | 1 | . | . | . | . |
| Breast-BR30T | . | 1 | . | . | . | . | . | . | . | . | 1 | . | . | . | . | . | . |
| Breast-BR31T | . | 1 | . | . | . | . | . | . | . | . | 1 | . | . | . | . | . | . |
| Breast-BR32T | . | 1 | . | 1 | . | . | . | . | . | . | . | . | 1 | . | . | . | . |
| Breast-BR34T | . | 1 | . | ? | . | . | . | . | . | . | 1 | . | . | . | . | . | . |
| Breast-BR36T | . | 1 | . | ? | . | . | . | . | . | . | 1 | . | . | . | . | . | . |
| Breast-BR37T | . | 1 | . | . | . | . | . | . | . | . | 1 | . | . | . | . | . | . |
| Breast-BR38T | . | 1 | . | . | . | . | . | . | . | . | 1 | . | . | . | . | . | . |
| Breast-BR39T | . | 1 | . | . | . | . | ? | . | . | . | 1 | . | . | . | . | . | . |
| Breast-BR41T | . | 1 | . | ? | . | . | . | . | . | . | 1 | . | . | . | . | . | . |
| Breast-BR46T | . | 1 | . | 1 | . | . | 1 | . | . | . | . | . | 1 | . | . | . | . |
| Breast-BR6T | . | 1 | . | . | . | . | . | . | . | . | 1 | . | . | . | . | . | . |
| Breast-BR8T | . | 1 | . | . | . | . | . | . | . | . | 1 | . | . | . | . | . | . |
| Breast-BRU1 | . | 1 | . | . | . | . | . | . | . | . | 1 | . | . | . | . | . | . |
| Breast-BRU16 | . | ? | . | . | . | ? | . | . | . | . | . | . | 1 | . | . | . | . |
| Breast-BRUX19 | . | . | . | . | . | . | . | . | . | . | . | . | 1 | . | . | . | . |
| Breast-BRUX7 | . | . | . | . | . | . | 1 | . | . | . | . | 1 | . | . | . | . | . |
| Breast-BRUX8 | . | 1 | . | . | . | . | . | . | . | . | 1 | . | . | . | . | . | . |

| | | | | | | | |
|---|---|---|---|---|---|---|---|
| SUMMARY | 19 | 1 | 6 | 26 | 73 | 4 | 23 |

TABLE 12-3
Predicted tissue type for each colorectal tumor sample in Su et al.

| SAMPLE (CO) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Colorectum-CO14T | . | . | 1 | . | . | . | . | . | . | . | 1 | . | . | . | . | . | . |
| Colorectum-CO15T | . | . | 1 | . | . | . | . | . | . | . | 1 | . | . | . | . | . | . |
| Colorectum-CO20T | . | . | 1 | . | . | . | . | . | . | . | 1 | . | . | . | . | . | . |
| Colorectum-CO21T | . | . | 1 | . | . | . | . | . | . | . | 1 | . | . | . | . | . | . |
| Colorectum-CO23T | . | . | 1 | . | . | . | . | . | . | . | 1 | . | . | . | . | . | . |
| Colorectum-CO24T | . | . | 1 | . | . | . | . | . | . | . | 1 | . | . | . | . | . | . |
| Colorectum-CO27T | . | . | 1 | . | . | . | . | . | . | . | 1 | . | . | . | . | . | . |
| Colorectum-CO30T | . | . | 1 | . | . | . | . | . | . | . | 1 | . | . | . | . | . | . |
| Colorectum-CO32T | . | . | 1 | ? | . | . | . | . | . | . | 1 | . | . | . | . | . | . |
| Colorectum-CO40T | . | . | 1 | 1 | . | . | . | . | . | . | . | . | 1 | . | . | . | . |
| Colorectum-CO42T | . | . | 1 | . | . | . | . | . | . | . | 1 | . | . | . | . | . | . |
| Colorectum-CO43T | . | . | 1 | . | . | . | . | . | . | . | 1 | . | . | . | . | . | . |
| Colorectum-CO44T | . | . | . | . | . | . | 1 | . | . | . | . | 1 | . | . | . | . | . |
| Colorectum-CO49T | . | . | 1 | . | . | . | . | . | . | . | 1 | . | . | . | . | . | . |
| Colorectum-CO51T | . | . | 1 | . | . | . | . | . | . | . | 1 | . | . | . | . | . | . |
| Colorectum-CO56T | . | . | 1 | 1 | . | . | . | . | . | . | . | . | 1 | . | . | . | . |
| Colorectum-CO5T | . | . | 1 | . | . | . | . | . | . | . | 1 | . | . | . | . | . | . |
| Colorectum-CO61T | . | . | 1 | . | . | . | . | . | ? | . | 1 | . | . | . | . | . | . |
| Colorectum-CO7T | . | . | 1 | . | . | . | . | . | . | . | 1 | . | . | . | . | . | . |
| Colorectum-CO8T | . | ? | 1 | . | . | . | . | . | . | . | 1 | . | . | . | . | . | . |
| Colorectum-CO9T | . | . | 1 | . | . | . | . | . | . | . | 1 | . | . | . | . | . | . |
| Colorectum-COU12 | . | 1 | ? | . | . | . | . | . | . | . | . | 1 | . | . | . | . | . |
| Colorectum-COU6 | . | . | 1 | ? | . | . | . | . | . | . | 1 | . | . | . | . | . | . |

| | | | | | | | |
|---|---|---|---|---|---|---|---|
| SUMMARY | 19 | 2 | 2 | 23 | 83 | 9 | 9 |

TABLE 12-4
Predicted tissue type for each gastroesophagus sample in Su et al.

| | | | | | | | | | | | | | | | % | % | % |
|---|---|---|---|---|---|---|---|---|---|---|---|---|---|---|---|---|---|
| SAMPLE (GA) | BL | BR | CO | GA | KI | LI | LU | OV | PA | PR | COR | INCOR | IND | TOT | COR | INCOR | IND |
| Gastroesophagus-GA102X | . | . | 1 | 1 | . | . | . | . | . | . | . | . | 1 | . | . | . | . |
| Gastroesophagus-GA116X | . | . | 1 | 1 | . | . | . | . | . | . | . | . | 1 | . | . | . | . |
| Gastroesophagus-GA18T | . | . | . | 1 | . | . | . | . | . | . | 1 | . | . | . | . | . | . |
| Gastroesophagus-GA280 | . | ? | . | ? | . | . | 1 | . | . | . | . | 1 | . | . | . | . | . |
| Gastroesophagus-GA2T | . | . | ? | 1 | . | . | . | . | . | . | 1 | . | . | . | . | . | . |
| Gastroesophagus-GA3T | . | . | ? | 1 | . | . | . | . | . | . | 1 | . | . | . | . | . | . |
| Gastroesophagus-GA46T | . | ? | 1 | 1 | . | . | 1 | . | . | . | . | . | 1 | . | . | . | . |
| Gastroesophagus-GA5T | . | . | ? | 1 | . | . | . | . | . | . | 1 | . | . | . | . | . | . |
| Gastroesophagus-GA6T | . | . | . | 1 | . | . | . | . | . | . | 1 | . | . | . | . | . | . |
| Gastroesophagus-GA8T | . | . | . | 1 | . | . | . | . | . | . | 1 | . | . | . | . | . | . |
| Gastroesophagus-GA9T | . | . | . | 1 | . | . | . | . | . | . | 1 | . | . | . | . | . | . |
| Gastroesophagus-GAU3 | . | . | . | . | . | . | 1 | . | . | . | . | 1 | . | . | . | . | . |

| | | | | | | | |
|---|---|---|---|---|---|---|---|
| SUMMARY | 7 | 2 | 3 | 12 | 58 | 17 | 25 |

TABLE 12-5
Predicted tissue type for each kidney sample in Su et al.

| SAMPLE (KI) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Kidney-KI16T | . | . | . | . | 1 | . | . | . | . | . | 1 | . | . | . | . | . | . |
| Kidney-KI17T | . | . | . | . | 1 | . | . | . | . | . | 1 | . | . | . | . | . | . |
| Kidney-KI18T | . | . | . | . | 1 | . | . | . | . | . | 1 | . | . | . | . | . | . |
| Kidney-KI19T | . | . | . | . | 1 | . | . | . | . | . | 1 | . | . | . | . | . | . |
| Kidney-KI1T | . | . | . | . | 1 | . | . | . | . | . | 1 | . | . | . | . | . | . |
| Kidney-KI20T | . | . | . | . | 1 | . | . | . | . | . | 1 | . | . | . | . | . | . |
| Kidney-KI22T | . | . | . | . | 1 | . | . | . | . | . | 1 | . | . | . | . | . | . |
| Kidney-KI2T | . | . | . | . | 1 | . | . | . | . | . | 1 | . | . | . | . | . | . |
| Kidney-KI3T | . | . | . | . | 1 | . | . | . | . | . | 1 | . | . | . | . | . | . |
| Kidney-KI4T | . | . | . | . | 1 | . | . | . | . | . | 1 | . | . | . | . | . | . |
| Kidney-KIUX14 | . | . | . | . | . | . | . | . | . | . | . | . | 1 | . | . | . | . |

| | | | | | | | |
|---|---|---|---|---|---|---|---|
| SUMMARY | 10 | 0 | 1 | 11 | 91 | 0 | 9 |

TABLE 12-6
Predicted tissue type for each lung adenocarcinoma sample in Su et al.

| SAMPLE (LU) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Lung-Adeno-LA17T | . | . | . | . | . | . | . | . | . | . | . | . | 1 | . | . | . | . |
| Lung-Adeno-LA18T | . | . | . | . | . | . | 1 | . | . | . | 1 | . | . | . | . | . | . |
| Lung-Adeno-LA20T | . | . | . | 1 | . | . | 1 | . | . | . | . | . | 1 | . | . | . | . |
| Lung-Adeno-LA31T | . | . | . | . | . | . | 1 | . | . | . | 1 | . | . | . | . | . | . |
| Lung-Adeno-LA33T | . | . | . | . | . | . | 1 | . | . | . | 1 | . | . | . | . | . | . |
| Lung-Adeno-LA34T | . | . | . | . | . | . | 1 | . | . | . | 1 | . | . | . | . | . | . |
| Lung-Adeno-LA39T | . | . | . | . | . | . | 1 | . | . | . | 1 | . | . | . | . | . | . |
| Lung-Adeno-LA40T | . | . | . | . | . | . | 1 | . | . | . | 1 | . | . | . | . | . | . |
| Lung-Adeno-LA44T | . | . | . | . | . | . | 1 | . | . | . | 1 | . | . | . | . | . | . |
| Lung-Adeno-LA5T | . | . | . | ? | . | . | 1 | . | . | . | 1 | . | . | . | . | . | . |
| Lung-Adeno-LA6T | . | . | . | . | . | . | 1 | . | . | . | 1 | . | . | . | . | . | . |
| Lung-Adeno-LA8T | . | . | . | . | . | . | 1 | . | . | . | 1 | . | . | . | . | . | . |
| Lung-Adeno-LAU17 | ? | . | . | . | . | . | 1 | . | . | . | 1 | . | . | . | . | . | . |
| Lung-Adeno-LAUX4 | . | . | . | . | . | . | 1 | . | . | . | 1 | . | . | . | . | . | . |

| | | | | | | | |
|---|---|---|---|---|---|---|---|
| SUMMARY | 12 | 0 | 2 | 14 | 86 | 0 | 14 |

TABLE 12-7
Predicted tissue type for each liver sample in Su et al.

| SAMPLE (LI) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Liver-LI11T | . | . | . | . | . | 1 | . | . | . | . | 1 | . | . | . | . | . | . |
| Liver-LI13T | . | . | . | . | . | 1 | . | . | . | . | 1 | . | . | . | . | . | . |
| Liver-LI130T | . | . | . | . | . | 1 | 1 | . | . | . | . | . | 1 | . | . | . | . |
| Liver-LI132T | . | . | . | . | . | 1 | . | . | . | . | 1 | . | . | . | . | . | . |
| Liver-LI134T | . | ? | . | . | . | 1 | . | . | . | . | 1 | . | . | . | . | . | . |
| Liver-LI135T | . | . | . | . | . | 1 | . | . | . | . | 1 | . | . | . | . | . | . |
| Liver-LIU9 | . | . | . | . | . | ? | . | . | . | . | . | . | 1 | . | . | . | . |

| | | | | | | | |
|---|---|---|---|---|---|---|---|
| SUMMARY | 5 | 0 | 2 | 7 | 71 | 0 | 29 |

TABLE 12-8
Predicted tissue type for each lung squamous cell carcinoma in Su et al.

| SAMPLE (LU) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Lung-Sarcoma-LS11T | . | . | . | . | . | . | 1 | . | . | . | 1 | . | . | . | . | . | . |
| Lung-Sarcoma-LS12T | . | . | . | . | . | . | ? | . | . | . | . | . | 1 | . | . | . | . |
| Lung-Sarcoma-LS13T | . | ? | . | . | . | . | ? | . | . | . | . | . | 1 | . | . | . | . |
| Lung-Sarcoma-LS14T | . | . | . | . | . | . | 1 | . | . | . | 1 | . | . | . | . | . | . |
| Lung-Sarcoma-LS19T | . | . | . | . | . | . | 1 | . | . | . | 1 | . | . | . | . | . | . |
| Lung-Sarcoma-LS24T | . | . | . | ? | . | . | . | . | . | . | . | . | 1 | . | . | . | . |
| Lung-Sarcoma-LS25T | . | . | . | . | . | . | 1 | . | . | . | 1 | . | . | . | . | . | . |
| Lung-Sarcoma-LS26T | . | . | . | . | . | . | 1 | . | . | . | 1 | . | . | . | . | . | . |
| Lung-Sarcoma-LS30T | . | . | . | . | . | . | 1 | . | . | . | 1 | . | . | . | . | . | . |
| Lung-Sarcoma-LS36T | . | . | . | . | . | . | 1 | . | . | . | 1 | . | . | . | . | . | . |
| Lung-Sarcoma-LS41T | . | . | . | . | . | . | 1 | . | . | . | 1 | . | . | . | . | . | . |
| Lung-Sarcoma-LS7T | . | . | . | . | . | . | 1 | . | . | . | 1 | . | . | . | . | . | . |
| Lung-Sarcoma-LSU19 | . | . | . | . | . | . | 1 | . | . | . | 1 | . | . | . | . | . | . |
| Lung-Sarcoma-LSU2 | . | . | . | . | . | . | 1 | . | . | . | 1 | . | . | . | . | . | . |

| | | | | | | | |
|---|---|---|---|---|---|---|---|
| SUMMARY | 11 | 0 | 3 | 14 | 79 | 0 | 21 |

TABLE 12-9
Predicted tissue type for each ovary sample in Su et al.

| SAMPLE (OV) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Ovary-OV16T | . | . | . | . | . | . | . | 1 | . | . | 1 | . | . | . | . | . | . |
| Ovary-OV1AT | . | . | . | . | . | . | ? | 1 | . | . | 1 | . | . | . | . | . | . |
| Ovary-OV21T | . | . | . | . | . | . | . | 1 | . | . | 1 | . | . | . | . | . | . |
| Ovary-OV23T | . | . | . | . | . | . | . | 1 | . | . | 1 | . | . | . | . | . | . |
| Ovary-OV27T | . | . | . | . | . | . | . | 1 | . | . | 1 | . | . | . | . | . | . |
| Ovary-OV2AT | . | . | . | . | . | . | . | 1 | . | . | 1 | . | . | . | . | . | . |
| Ovary-OV3T | . | . | . | . | . | . | . | 1 | . | . | 1 | . | . | . | . | . | . |
| Ovary-OV7T | . | . | . | . | . | . | . | 1 | . | . | 1 | . | . | . | . | . | . |
| Ovary-OV8T | . | . | . | . | . | . | . | . | . | . | . | . | 1 | . | . | . | . |
| Ovary-OVR1 | . | . | . | . | . | . | . | 1 | . | . | 1 | . | . | . | . | . | . |
| Ovary-OVR10 | . | . | . | . | . | . | . | 1 | . | . | 1 | . | . | . | . | . | . |
| Ovary-OVR11 | . | . | . | . | . | . | . | 1 | . | . | 1 | . | . | . | . | . | . |
| Ovary-OVR12 | . | . | . | . | . | . | . | 1 | . | . | 1 | . | . | . | . | . | . |
| Ovary-OVR13 | . | . | . | . | . | . | . | 1 | . | . | 1 | . | . | . | . | . | . |
| Ovary-OVR16 | . | . | . | . | . | . | . | 1 | . | . | 1 | . | . | . | . | . | . |
| Ovary-OVR19 | . | . | . | . | . | . | . | 1 | . | . | 1 | . | . | . | . | . | . |
| Ovary-OVR2 | . | . | . | . | . | . | . | 1 | . | . | 1 | . | . | . | . | . | . |
| Ovary-OVR22 | . | . | . | . | . | . | . | 1 | . | . | 1 | . | . | . | . | . | . |
| Ovary-OVR26 | . | . | . | . | . | . | . | 1 | . | . | 1 | . | . | . | . | . | . |
| Ovary-OVR27 | . | . | . | . | . | . | . | 1 | . | . | 1 | . | . | . | . | . | . |
| Ovary-OVR28 | . | . | . | . | . | . | . | 1 | . | . | 1 | . | . | . | . | . | . |
| Ovary-OVR5 | . | . | . | . | . | . | . | 1 | . | . | 1 | . | . | . | . | . | . |
| Ovary-OVR8 | . | . | . | . | . | . | . | 1 | . | . | 1 | . | . | . | . | . | . |
| Ovary-OVU11 | . | . | . | . | . | . | . | 1 | . | . | 1 | . | . | . | . | . | . |
| Ovary-OVU7 | . | . | . | . | . | . | . | 1 | . | . | 1 | . | . | . | . | . | . |
| Ovary-OVU8 | . | . | . | . | . | . | . | 1 | . | . | 1 | . | . | . | . | . | . |
| Ovary-OVUX20 | . | . | . | . | . | . | ? | 1 | . | . | 1 | . | . | . | . | . | . |

| | | | | | | | |
|---|---|---|---|---|---|---|---|
| SUMMARY | 26 | 0 | 1 | 27 | 96 | 0 | 4 |

TABLE 12-10
Predicted tissue type for each pancreas sample in Su et al.

| SAMPLE (PA) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Pancreas-PA11T | . | . | . | . | . | . | . | . | 1 | . | 1 | . | . | . | . | . | . |
| Pancreas-PA16BT | . | . | . | . | . | . | . | . | 1 | . | 1 | . | . | . | . | . | . |
| Pancreas-PA17T | . | . | . | . | . | . | . | . | 1 | . | 1 | . | . | . | . | . | . |
| Pancreas-PA22T | . | . | . | . | . | . | . | . | 1 | . | 1 | . | . | . | . | . | . |
| Pancreas-PA23T | . | . | . | . | . | . | . | . | . | . | . | . | 1 | . | . | . | . |
| Pancreas-PA8T | . | . | . | . | . | . | . | . | 1 | . | 1 | . | . | . | . | . | . |

| | | | | | | | |
|---|---|---|---|---|---|---|---|
| SUMMARY | 5 | 0 | 1 | 6 | 83 | 0 | 17 |

TABLE 12-11
Predicted tissue type for each prostate sample in Su et al.

| SAMPLE (PR) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| Prostate-PR1 | . | . | . | . | . | . | . | . | . | 1 | 1 | . | . | . | . | . | . |
| Prostate-PR10 | . | . | . | . | . | . | . | . | . | 1 | 1 | . | . | . | . | . | . |
| Prostate-PR11 | . | . | . | . | . | . | . | . | . | 1 | 1 | . | . | . | . | . | . |
| Prostate-PR12 | . | . | . | . | . | . | . | . | . | 1 | 1 | . | . | . | . | . | . |
| Prostate-PR13BT | . | . | . | . | . | . | . | . | . | 1 | 1 | . | . | . | . | . | . |
| Prostate-PR16 | . | . | . | . | . | . | . | . | . | 1 | 1 | . | . | . | . | . | . |
| Prostate-PR17 | . | . | . | . | . | . | . | . | . | 1 | 1 | . | . | . | . | . | . |
| Prostate-PR19T | . | . | . | . | . | . | . | . | . | 1 | 1 | . | . | . | . | . | . |
| Prostate-PR21T | . | . | . | . | . | . | . | . | . | 1 | 1 | . | . | . | . | . | . |
| Prostate-PR22 | . | . | . | . | . | . | . | . | . | 1 | 1 | . | . | . | . | . | . |
| Prostate-PR23 | . | . | . | . | . | . | . | . | . | 1 | 1 | . | . | . | . | . | . |
| Prostate-PR24T | . | . | . | . | . | . | . | . | . | 1 | 1 | . | . | . | . | . | . |
| Prostate-PR26 | . | . | . | . | . | . | . | . | . | 1 | 1 | . | . | . | . | . | . |
| Prostate-PR27T | . | . | . | . | . | . | . | . | . | 1 | 1 | . | . | . | . | . | . |
| Prostate-PR29T | . | . | . | . | . | . | . | . | . | 1 | 1 | . | . | . | . | . | . |
| Prostate-PR3 | . | . | . | . | . | . | . | . | . | 1 | 1 | . | . | . | . | . | . |
| Prostate-PR30 | . | . | . | . | . | . | . | . | . | 1 | 1 | . | . | . | . | . | . |
| Prostate-PR31 | . | . | . | . | . | . | . | . | . | 1 | 1 | . | . | . | . | . | . |
| Prostate-PR4 | . | . | . | . | . | . | . | . | . | 1 | 1 | . | . | . | . | . | . |
| Prostate-PR5T | . | . | . | . | . | . | . | . | . | 1 | 1 | . | . | . | . | . | . |
| Prostate-PR6 | . | . | . | . | . | . | . | . | . | 1 | 1 | . | . | . | . | . | . |
| Prostate-PR7T | . | . | . | . | . | . | . | . | . | 1 | 1 | . | . | . | . | . | . |
| Prostate-PR8T | . | . | . | . | . | . | . | . | . | 1 | 1 | . | . | . | . | . | . |
| Prostate-PR9T | . | . | . | . | . | . | . | . | . | 1 | 1 | . | . | . | . | . | . |
| Prostate-PRU40 | . | . | . | . | . | . | . | . | . | 1 | 1 | . | . | . | . | . | . |
| Prostate-PRU41 | . | . | . | . | . | . | . | . | . | 1 | 1 | . | . | . | . | . | . |

| | | | | | | | |
|---|---|---|---|---|---|---|---|
| SUMMARY | 26 | 0 | 0 | 26 | 100 | 0 | 0 |

Table 13 summarizes the results of this experiment by summarizing classifications by tissue type. In Table 13, #Samples is the number of biological specimens 58 tested, #COR is the number of correctly identified biological specimens for the corresponding origin site, #INCOR is the percentage of incorrectly identified biological specimens for the corresponding origin site, #IND is the number of indeterminates.

TABLE 13
Summary of classification results for Su et al. data based
on tissue type Model Summary

| Abbr | Origin Site | #Samples | #COR | #INCOR | #IND |
|---|---|---|---|---|---|
| BL | Bladder | 8 | 6 | 0 | 2 |
| BR | Breast | 26 | 19 | 1 | 6 |
| CO | Colorectal | 23 | 19 | 2 | 2 |
| GA | Gastroesophagus | 12 | 7 | 2 | 3 |
| KI | Kidney | 11 | 10 | 0 | 1 |
| LI | Liver | 7 | 5 | 0 | 2 |
| LU | Lung | 28 | 23 | 0 | 5 |
| OV | Ovary | 27 | 26 | 0 | 1 |
| PA | Pancreas | 6 | 5 | 0 | 1 |
| PR | Prostate | 26 | 26 | 0 | 0 |
| | TOTALS | 174 | 146 | 5 | 23 |

Table 14 shows the percent correct, percent incorrect, and percent indeterminate for each tissue type using the Su-Hampton 2001 model for the Su et al. data that were computed using the methods of the present invention.

TABLE 14
Summary of classification results for Su et al.
data using the methods of the present invention.

| Abbr | Origin Site | % Correct | % Incorrect | % Indeterminate |
|---|---|---|---|---|
| BL | Bladder | 75 | 0 | 25 |
| BR | Breast | 73 | 3 | 23 |
| CO | Colorectal | 82 | 8 | 8 |
| GA | Gastroesophagus | 58 | 16 | 25 |
| KI | Kidney | 90 | 0 | 9 |
| LI | Liver | 71 | 0 | 28 |
| LU | Lung | 82 | 0 | 17 |
| OV | Ovary | 96 | 0 | 3 |
| PA | Pancreas | 83 | 0 | 16 |
| PR | Prostate | 100 | 0 | 0 |
| | OVERALL | 84 | 3 | 13 |

Using the techniques described in Section 5.1 and 5.2, the calculated sets 72 (the Su-Hampton 2001 model) correctly identified 146 of the 174 tissue samples used in Su et al. The Su-Hampton 2001 model declared as indeterminate 23 samples that could not be classified with confidence. There were five samples that were incorrectly classified. This result compares favorably to Su et al., where seven samples were incorrectly classified.

### 6.2 Cross Validation—Cancer of Unknown Primary

The Su-Hampton 2001 developed in Section 6.1 was tested using data obtained by Bhattachaijee et al., Proceeding of the National Academy of Science 98, p. 13790, 2001. Bhattachaijee et al. used gene expression data to provide evidence that subclasses of human lung carcinomas present distinct genetic markers.

Step 302.

The samples used in Bhattachaijee et al came from cancerous lung tumors of four types. The samples included 127 adenocarcinomas, 49 of which had duplicate tissue samples for a total of 176 adenocarcinomas samples. The samples further included 12 samples originally thought to be lung adenocarcinomas, but were identified by Bhattachaxjee et al. to most likely represent metastatic adenocarcinomas from the colon. Two of these had duplicate tissue samples for a total of 14 metastatic colorectal samples. The samples further included 21 lung squamous cell carcinomas, 20 pulmonary carcinoids, 6 small-cell lung carcinomas, and 17 normal lung specimens for a total of 254 samples.

Because the Su-Hampton 2001 model does not have specific ratios for pulmonary carcinoids or small-cell lung carcinomas, these samples were not used in the cross-validation. Also, since it was not known beforehand that the metastatic colorectal samples were not lung samples, the samples that were metastatic colon samples were reported as if they were primary lung adenocarcinoma samples. In total, 211 samples were used for the Su-Hampton 2001 cross-validation: 190 adenocarcinomas, which includes 14 metastatic colorectal samples, and 21 squamous cell carcinomas.

In Bhattacharjee et al., total RNA extracted from samples was used to generate cRNA target which was subsequently hybridized to human U95A oligonucleotide probe arrays (Affymetrix, Santa Clara, Calif.) in accordance with Golub et al., 1999, Science 286, p. 531.

Step 304.

One data file that contained the gene expression data of the tissue was created for each sample. The expression value for each gene in each respective file was divided by the median gene expression value of the respective file in order to standardize gene expression values.

Step 306.

Each ratio determined by each set 72 of the Su-Hampton 2001 model returns one of three results: positive, negative, or indeterminate. Eleven tests were run for each biological specimen 58 from Bhattachaxjee et al. Each test consisted of calculating each ratio defined by a cellular constituent pair in a given set 72 and determining whether the ratio was positive, negative, or indeterminate.

Step 308.

In step 306, Su-Hampton 2001 ratios were computed for each biological specimen 58 from Bhattachatjee et al. and then classified as positive, negative, or indeterminate. In step 308, the eleven ratios sets calculated for each biological specimen from Bhattacharjee et al. were characterized in accordance with equations (I), (II) and (III) from Section 6.1, above.

Step 310.

In Table 15, the predicted tissue type for each sample in Bhattachairjee et al. is described. These predictions were made using the sets 72 calculated above (i.e., the Su-Hampton 2001 model). In Table 15, a “1” in a tissue type column indicates a positive result for that tissue type, “?” indicates an indeterminate result, and a “.” indicates a negative result. To the right of the eleven columns representing the eleven possible tissue types are columns representing the final classification of each sample. These final classifications are correct (COR), incorrect (INCOR), or indefinite (IND). Also reported is total (TOT), percent correct (% COR), percent incorrect (% INCOR), and percent indeterminate (% IND).

TABLE 15-1
Bhattacharjee et al. colorectal carcinomas analyzed using the Su-Hampton 2001 model

| | | | | | | | | | | | | | | | % | % | % |
|---|---|---|---|---|---|---|---|---|---|---|---|---|---|---|---|---|---|
| SAMPLE (CO) | BL | BR | CO | GA | KI | LI | LU | OV | PA | PR | COR | INCOR | IND | TOT | COR | INCOR | IND |
| AD043T2_A7_1_LA | . | . | 1 | . | . | . | . | . | . | . | 1 | . | . | . | . | . | . |
| AD202T2_A139_4_LA | . | ? | 1 | . | . | . | 1 | . | . | . | . | . | 1 | . | . | . | . |
| AD218T1_A147_4_LA | . | . | . | 1 | . | . | 1 | . | . | . | . | . | 1 | . | . | . | . |
| AD221T1_A148_4_LA | . | . | 1 | . | . | . | . | . | . | . | 1 | . | . | . | . | . | . |
| AD241T1_A160_4_LA | . | . | 1 | . | . | . | 1 | . | . | . | . | . | 1 | . | . | . | . |
| AD285T2_A263_10_LA | . | . | 1 | . | . | . | 1 | . | . | . | . | . | 1 | . | . | . | . |
| AD314T1_A269_10_LA | . | ? | 1 | . | . | . | . | . | . | . | 1 | . | . | . | . | . | . |
| AD320T1_A272_10_LA | . | . | 1 | ? | . | . | 1 | . | . | . | . | . | 1 | . | . | . | . |
| AD338T1_A121_3_LA | . | . | . | . | . | . | 1 | . | . | . | . | 1 | . | . | . | . | . |
| AD340T1_A122_3_LA | . | . | 1 | ? | . | . | 1 | . | . | . | . | . | 1 | . | . | . | . |
| AD384T2_A288_10_LA | . | . | 1 | ? | . | . | 1 | . | . | . | . | . | 1 | . | . | . | . |
| AD384T1_A120_3_LA | . | . | 1 | . | . | . | 1 | . | . | . | . | . | 1 | . | . | . | . |
| ADA5T1_A387_7_LA | . | . | 1 | . | . | . | . | . | . | . | 1 | . | . | . | . | . | . |
| ADA7T1_A388_7_LA | . | . | 1 | . | . | . | . | . | . | . | 1 | . | . | . | . | . | . |

| | | | | | | | |
|---|---|---|---|---|---|---|---|
| SUMMARY | 5 | 1 | 8 | 14 | 36 | 7 | 57 |

TABLE 15-2
Bhattacharjee et al. lung carcinomas analyzed using the Su-Hampton 2001 model

| SAMPLE (LU) | BL | BR | CO | GA | KI | LI | LU | OV | PA | PR | COR | INCOR | IND | TOT | % COR | % INCOR | % IND |
|---|---|---|---|---|---|---|---|---|---|---|---|---|---|---|---|---|---|
| ADA1T1_A383_7_LA | . | . | . | . | . | . | 1 | . | . | . | 1 | . | . | . | . | . | . |
| ADA10T1_A389_7_LA | . | . | . | . | . | . | 1 | . | . | . | 1 | . | . | . | . | . | . |
| AD111T2_A8_1_LA | . | . | . | . | . | . | 1 | . | . | . | 1 | . | . | . | . | . | . |
| AD114T1_A9_1_LA | . | . | . | . | . | . | 1 | . | . | . | 1 | . | . | . | . | . | . |
| AD114T2_A10_1_LA | . | . | . | . | . | . | 1 | . | . | . | 1 | . | . | . | . | . | . |
| AD115T1_A12_1_LA | . | . | . | . | . | . | 1 | . | . | . | 1 | . | . | . | . | . | . |
| AD115T2_A245_10_LA | . | . | . | . | . | . | 1 | . | . | . | 1 | . | . | . | . | . | . |
| AD118T1_A13_1_LA | . | . | . | . | . | . | 1 | . | . | . | 1 | . | . | . | . | . | . |
| AD119T3_A195_8_LA | . | . | . | . | . | . | 1 | . | . | . | 1 | . | . | . | . | . | . |
| AD120T1_A226_8_LA | . | . | . | . | . | . | 1 | . | . | . | 1 | . | . | . | . | . | . |
| AD120T2_A196_8_LA | . | . | . | 1 | . | . | 1 | . | . | . | . | . | 1 | . | . | . | . |
| AD122T3_A197_8_LA | . | . | . | . | . | . | 1 | . | . | . | 1 | . | . | . | . | . | . |
| AD123T1_A25_1_LA | . | . | . | . | . | . | 1 | . | . | . | 1 | . | . | . | . | . | . |
| AD123T2_A198_8_LA | . | . | . | . | . | . | 1 | . | . | . | 1 | . | . | . | . | . | . |
| AD127T1_A14_1_LA | . | . | . | . | . | . | 1 | . | . | . | 1 | . | . | . | . | . | . |
| AD130T1_A1_1_LA | . | . | . | . | . | . | 1 | . | . | . | 1 | . | . | . | . | . | . |
| AD131T1_A15_1_LA | . | . | . | . | . | . | ? | . | . | . | . | . | 1 | . | . | . | . |
| AD131T1_A200_8_LA | . | . | . | . | . | . | . | . | . | . | . | . | 1 | . | . | . | . |
| AD136T2_A201_8_LA | . | ? | . | . | . | . | 1 | . | . | . | 1 | . | . | . | . | . | . |
| ADA15T1_A390_7_LA | . | . | . | . | . | . | 1 | . | . | . | 1 | . | . | . | . | . | . |
| AD157T1_A246_10_LA | . | . | . | ? | . | . | ? | . | . | . | . | . | 1 | . | . | . | . |
| AD157T2_A26_1_LA | . | . | . | . | . | . | 1 | . | . | . | 1 | . | . | . | . | . | . |
| AD158T1_A247_10_LA | . | . | . | . | . | . | 1 | . | . | . | 1 | . | . | . | . | . | . |
| AD158T2_A17_1_LA | . | . | . | . | . | . | 1 | . | . | . | 1 | . | . | . | . | . | . |
| AD159T1_A229_8_LA | . | ? | . | . | . | . | 1 | . | . | . | 1 | . | . | . | . | . | . |
| ADA16T2_A391_7_LA | . | . | . | . | . | . | 1 | . | . | . | 1 | . | . | . | . | . | . |
| AD162T2_A230_8_LA | . | . | . | . | . | . | 1 | . | . | . | 1 | . | . | . | . | . | . |
| AD163T1_A203_8_LA | . | ? | . | . | . | . | . | . | . | . | . | . | 1 | . | . | . | . |
| AD163T3_A205_8_LA | . | ? | . | . | . | . | . | . | . | . | . | . | 1 | . | . | . | . |
| AD164T1a_A206_8_LA | . | . | . | 1 | . | . | 1 | . | . | . | . | . | 1 | . | . | . | . |
| AD164T2_A208_8_LA | . | . | . | . | . | . | 1 | . | . | . | 1 | . | . | . | . | . | . |
| AD167T1_A210_8_LA | . | . | . | ? | . | . | 1 | . | . | . | 1 | . | . | . | . | . | . |
| AD167T2_A249_10_LA | . | . | . | ? | . | . | 1 | . | . | . | 1 | . | . | . | . | . | . |
| AD169T2_A211_8_LA | . | . | . | . | . | . | . | . | . | . | . | . | 1 | . | . | . | . |
| AD169T3_A250_10_LA | . | . | . | . | . | . | . | . | . | . | . | . | 1 | . | . | . | . |
| AD170T1_A251_10_LA | . | . | . | . | . | . | 1 | . | . | . | 1 | . | . | . | . | . | . |
| AD170T2_A5_8_LA | . | ? | . | . | . | . | 1 | . | . | . | 1 | . | . | . | . | . | . |
| AD172T2_A213_8_LA | . | . | . | . | . | . | 1 | . | . | . | 1 | . | . | . | . | . | . |
| AD172T4_A252_10_LA | . | . | . | . | . | . | 1 | . | . | . | 1 | . | . | . | . | . | . |
| AD173T1a_A23_1_LA | . | . | . | . | . | . | 1 | . | . | . | 1 | . | . | . | . | . | . |
| AD177T1_A21_1_LA | . | . | . | . | . | . | 1 | . | . | . | 1 | . | . | . | . | . | . |
| AD178T2_A22_1_LA | . | . | . | . | . | . | 1 | . | . | . | 1 | . | . | . | . | . | . |
| AD178T3_A254_10_LA | . | . | . | . | . | . | 1 | . | . | . | 1 | . | . | . | . | . | . |
| AD179T1_A214_8_LA | . | . | . | . | . | . | 1 | . | . | . | 1 | . | . | . | . | . | . |
| AD179T2_A255_10_LA | . | . | . | . | . | . | 1 | . | . | . | 1 | . | . | . | . | . | . |
| ADA18T1_A392_7_LA | . | . | . | . | . | . | 1 | . | . | . | 1 | . | . | . | . | . | . |
| AD183T1_A6_8_LA | . | . | . | . | . | . | 1 | . | . | . | 1 | . | . | . | . | . | . |
| AD183T1_A215_1_LA | . | . | . | . | . | . | 1 | . | . | . | 1 | . | . | . | . | . | . |
| AD185T2_A232_8_LA | . | . | . | . | . | . | 1 | . | . | . | 1 | . | . | . | . | . | . |
| AD186T1_A27_1_LA | . | . | . | 1 | . | . | 1 | . | . | . | . | . | 1 | . | . | . | . |
| AD187T1_A11_1_LA | . | ? | . | . | . | . | 1 | . | . | . | 1 | . | . | . | . | . | . |
| AD187T2_A233_8_LA | . | ? | . | . | . | . | 1 | . | . | . | 1 | . | . | . | . | . | . |
| AD188T1_A216_8_LA | . | . | . | . | . | . | 1 | . | . | . | 1 | . | . | . | . | . | . |
| ADA19T1_A393_7_LA | . | . | . | . | . | . | 1 | . | . | . | 1 | . | . | . | . | . | . |
| ADA2T1_A384_7_LA | . | . | . | . | . | . | 1 | . | . | . | 1 | . | . | . | . | . | . |
| AD201T1_A138_4_LA | . | . | . | . | . | . | 1 | . | . | . | 1 | . | . | . | . | . | . |
| AD203T1_A140_4_LA | . | . | ? | . | . | . | 1 | . | . | . | 1 | . | . | . | . | . | . |
| AD203T2_A141_4_LA | . | . | . | . | . | . | 1 | . | . | . | 1 | . | . | . | . | . | . |
| AD207T1_A142_4_LA | . | . | . | . | . | . | 1 | . | . | . | 1 | . | . | . | . | . | . |
| AD208T1_A143_4_LA | . | . | . | . | . | . | 1 | . | . | . | 1 | . | . | . | . | . | . |
| AD210T1_A144_4_LA | . | . | . | . | . | . | 1 | . | . | . | 1 | . | . | . | . | . | . |
| AD212T1_A145_4_LA | . | . | . | . | . | . | 1 | . | . | . | 1 | . | . | . | . | . | . |
| AD213T1_A146_4_LA | . | . | . | . | . | . | 1 | . | . | . | 1 | . | . | . | . | . | . |
| AD224T1_A149_4_LA | . | . | . | . | . | . | 1 | . | . | . | 1 | . | . | . | . | . | . |
| AD225T1_A150_4_LA | . | . | . | 1 | . | . | 1 | . | . | . | . | . | 1 | . | . | . | . |
| AD226T2_A151_4_LA | . | . | . | . | . | . | 1 | . | . | . | 1 | . | . | . | . | . | . |
| AD228T2_A152_4_LA | . | . | . | . | . | . | 1 | . | . | . | 1 | . | . | . | . | . | . |
| AD228T3_A256_10_LA | . | . | . | . | . | . | 1 | . | . | . | 1 | . | . | . | . | . | . |
| AD230T1_A153_4_LA | . | . | . | . | . | . | 1 | . | . | . | 1 | . | . | . | . | . | . |
| AD232T1_A154_4_LA | . | . | . | . | . | . | 1 | . | . | . | 1 | . | . | . | . | . | . |
| AD234T1_A155_4_LA | . | . | . | . | . | . | 1 | . | . | . | 1 | . | . | . | . | . | . |
| AD236T1_A156_4_LA | . | . | . | . | . | . | 1 | . | . | . | 1 | . | . | . | . | . | . |
| AD238T2_A157_4_LA | . | . | . | . | . | . | 1 | . | . | . | 1 | . | . | . | . | . | . |
| AD239T1_A158_4_LA | . | . | . | . | . | . | 1 | . | . | . | 1 | . | . | . | . | . | . |
| AD240T1_A159_4_LA | . | . | . | 1 | . | . | 1 | . | . | . | . | . | 1 | . | . | . | . |
| AD243T1_A161_4_LA | . | . | . | . | . | . | 1 | . | . | . | 1 | . | . | . | . | . | . |
| AD243T2_A257_10_LA | . | . | . | . | . | . | 1 | . | . | . | 1 | . | . | . | . | . | . |
| AD247T1_A164_4_LA | . | . | . | . | . | . | 1 | . | . | . | 1 | . | . | . | . | . | . |
| AD249T1_A165_4_LA | . | . | . | . | . | . | 1 | . | . | . | 1 | . | . | . | . | . | . |
| AD250T1_A166_4_LA | . | . | . | . | . | . | 1 | . | . | . | 1 | . | . | . | . | . | . |
| AD252T1_A167_4_LA | . | . | . | . | . | . | 1 | . | . | . | 1 | . | . | . | . | . | . |
| AD253T1_A168_4_LA | . | . | . | . | . | . | 1 | . | . | . | 1 | . | . | . | . | . | . |
| AD255T1_A169_4_LA | . | . | . | . | . | . | 1 | . | . | . | 1 | . | . | . | . | . | . |
| AD255T1_A186_4_LA | . | . | . | . | . | . | 1 | . | . | . | 1 | . | . | . | . | . | . |
| AD255T1_A178_4_LA | . | . | . | . | . | . | 1 | . | . | . | 1 | . | . | . | . | . | . |
| AD258T1_A170_4_LA | . | . | . | . | . | . | 1 | . | . | . | 1 | . | . | . | . | . | . |
| AD258T2_A258_10_LA | . | . | . | . | . | . | 1 | . | . | . | 1 | . | . | . | . | . | . |
| AD258T1_A179_4_LA | . | . | . | . | . | . | ? | . | . | . | . | . | 1 | . | . | . | . |
| AD258T1_A187_4_LA | . | . | . | . | . | . | 1 | . | . | . | 1 | . | . | . | . | . | . |
| AD259T1_A171_4_LA | . | . | . | . | . | . | 1 | . | . | . | 1 | . | . | . | . | . | . |
| AD260T1_A172_4_LA | . | . | . | . | . | . | ? | . | . | . | . | . | 1 | . | . | . | . |
| AD260T1_A180_4_LA | . | . | . | . | . | . | 1 | . | . | . | 1 | . | . | . | . | . | . |
| AD261T1_A173_4_LA | . | . | . | . | . | . | . | . | . | . | . | . | 1 | . | . | . | . |
| AD262T1_A259_10_LA | . | . | . | . | . | . | 1 | . | . | . | 1 | . | . | . | . | . | . |
| AD262T1_A339_6_LA | . | . | . | . | . | . | 1 | . | . | . | 1 | . | . | . | . | . | . |
| AD266T1_A90_3_LA | . | . | . | . | . | . | 1 | . | . | . | 1 | . | . | . | . | . | . |
| AD267T1_A91_3_LA | . | . | . | . | . | . | 1 | . | . | . | 1 | . | . | . | . | . | . |
| AD268T1_A93_3_LA | . | . | . | ? | . | . | . | . | . | . | . | . | 1 | . | . | . | . |
| AD268T2_A262_10_LA | . | . | . | . | . | . | 1 | . | . | . | 1 | . | . | . | . | . | . |
| AD268T2_A189_4_LA | . | . | . | . | . | . | . | . | . | . | . | . | 1 | . | . | . | . |
| AD269T1_A94_3_LA | . | . | . | 1 | . | . | 1 | . | . | . | . | . | 1 | . | . | . | . |
| AD275T1_A95_3_LA | . | . | . | . | . | . | 1 | . | . | . | 1 | . | . | . | . | . | . |
| AD276T1_A96_3_LA | . | . | . | . | . | . | 1 | . | . | . | 1 | . | . | . | . | . | . |
| AD276T2_A190_4_LA | . | . | . | . | . | . | 1 | . | . | . | 1 | . | . | . | . | . | . |
| AD277T1_A97_3_LA | . | . | . | . | . | . | 1 | . | . | . | 1 | . | . | . | . | . | . |
| AD283T1_A99_3_LA | . | . | . | . | . | . | 1 | . | . | . | 1 | . | . | . | . | . | . |
| AD287T1_A101_3_LA | . | . | . | ? | . | . | . | . | . | . | . | . | 1 | . | . | . | . |
| AD294T1_A104_3_LA | . | . | . | ? | . | . | 1 | . | . | . | 1 | . | . | . | . | . | . |
| AD294T2_A191_4_LA | . | . | . | . | . | . | ? | . | . | . | . | . | 1 | . | . | . | . |
| AD295T1_A105_3_LA | . | . | . | . | . | . | 1 | . | . | . | 1 | . | . | . | . | . | . |
| AD296T1_A106_3_LA | . | . | . | . | . | . | ? | . | . | . | . | . | 1 | . | . | . | . |
| AD296T2_A264_10_LA | . | . | . | . | . | . | . | . | . | . | . | . | 1 | . | . | . | . |
| AD299T1_A235_8_LA | . | 1 | . | . | . | . | 1 | . | . | . | . | . | 1 | . | . | . | . |
| AD299T2_A236_8_LA | . | . | . | . | . | . | 1 | . | . | . | 1 | . | . | . | . | . | . |
| ADA3T1_A385_7_LA | . | . | . | . | . | . | ? | . | . | . | . | . | 1 | . | . | . | . |
| AD301T1_A237_8_LA | . | . | . | . | . | . | ? | . | . | . | . | . | 1 | . | . | . | . |
| AD301T1_A265_10_LA | . | . | . | . | . | . | 1 | . | . | . | 1 | . | . | . | . | . | . |
| AD302T3_A238_8_LA | . | . | . | . | . | . | 1 | . | . | . | 1 | . | . | . | . | . | . |
| AD302T4_A239_8_LA | . | . | . | . | . | . | 1 | . | . | . | 1 | . | . | . | . | . | . |
| AD304T1_A240_8_LA | . | . | . | . | . | . | 1 | . | . | . | 1 | . | . | . | . | . | . |
| AD305T1_A415_7_LA | . | . | . | . | . | . | 1 | . | . | . | 1 | . | . | . | . | . | . |
| AD308T1_A241_8_LA | . | . | . | . | . | . | 1 | . | . | . | 1 | . | . | . | . | . | . |
| AD309T1_A242_8_LA | . | . | . | . | . | . | . | . | . | . | . | . | 1 | . | . | . | . |
| ADA31_A289_10_LA | . | . | . | . | . | . | . | . | . | . | . | . | 1 | . | . | . | . |
| AD311T1_A266_10_LA | . | . | . | . | . | . | 1 | . | . | . | 1 | . | . | . | . | . | . |
| AD311T2_A267_10_LA | . | . | . | . | . | . | 1 | . | . | . | 1 | . | . | . | . | . | . |
| AD313T1_A268_10_LA | . | . | . | ? | . | . | 1 | . | . | . | 1 | . | . | . | . | . | . |
| AD315T1_A270_10_LA | . | . | . | . | . | . | 1 | . | . | . | 1 | . | . | . | . | . | . |
| AD317T1_A271_10_LA | . | ? | . | . | . | . | 1 | . | . | . | 1 | . | . | . | . | . | . |
| AD318T3_A107_3_LA | . | . | . | . | . | . | 1 | . | . | . | 1 | . | . | . | . | . | . |
| AD323T1_A273_10_LA | . | . | . | . | . | . | 1 | . | . | . | 1 | . | . | . | . | . | . |
| AD327T1_A276_10_LA | . | . | . | . | . | . | 1 | . | . | . | 1 | . | . | . | . | . | . |
| AD327T3_A277_10_LA | . | . | . | . | . | . | 1 | . | . | . | 1 | . | . | . | . | . | . |
| AD334T2_A280_10_LA | . | . | . | . | . | . | 1 | . | . | . | 1 | . | . | . | . | . | . |
| AD330T2_A279_10_LA | . | . | . | . | . | . | 1 | . | . | . | 1 | . | . | . | . | . | . |
| AD331T1_A219_8_LA | . | . | . | . | . | . | 1 | . | . | . | 1 | . | . | . | . | . | . |
| AD332T1_A220_8_LA | . | . | . | . | . | . | 1 | . | . | . | 1 | . | . | . | . | . | . |
| AD334T1_A221_8_LA | . | ? | . | . | . | . | ? | . | . | . | . | . | 1 | . | . | . | . |
| AD335T2_A281_10_LA | . | . | . | ? | . | . | 1 | . | . | . | 1 | . | . | . | . | . | . |
| AD335T1_A222_8_LA | . | . | . | . | . | . | 1 | . | . | . | 1 | . | . | . | . | . | . |
| AD338T1_A130_3_LA | . | . | . | . | . | . | ? | . | . | . | . | . | 1 | . | . | . | . |
| AD336T1_A223_8_LA | . | . | . | 1 | . | . | 1 | . | . | . | . | . | 1 | . | . | . | . |
| AD337T1_A224_8_LA | . | . | . | . | . | . | 1 | . | . | . | 1 | . | . | . | . | . | . |
| AD340T1_A131_3_LA | . | . | ? | . | . | . | 1 | . | . | . | 1 | . | . | . | . | . | . |
| AD341T1_A132_3_LA | . | . | . | . | . | . | . | . | . | . | . | . | 1 | . | . | . | . |
| AD341T1_A123_3_LA | . | . | . | . | . | . | . | . | . | . | . | . | 1 | . | . | . | . |
| AD346T1_A133_3_LA | . | . | . | . | . | . | 1 | . | . | . | 1 | . | . | . | . | . | . |
| AD346T1_A124_3_LA | . | . | . | . | . | . | 1 | . | . | . | 1 | . | . | . | . | . | . |
| AD347T1_A134_3_LA | . | . | . | . | . | . | 1 | . | . | . | 1 | . | . | . | . | . | . |
| AD347T1_A125_3_LA | . | . | . | . | . | . | ? | . | . | . | . | . | 1 | . | . | . | . |
| AD350T1_A135_3_LA | . | . | . | . | . | . | 1 | . | . | . | 1 | . | . | . | . | . | . |
| AD350T1_A126_3_LA | . | . | ? | . | . | . | 1 | . | . | . | 1 | . | . | . | . | . | . |
| AD360T2_A406_7_LA | . | . | . | . | . | . | . | . | . | . | . | . | 1 | . | . | . | . |
| AD351T1_A127_3_LA | . | . | . | . | . | . | . | . | . | . | . | . | 1 | . | . | . | . |
| AD352T1_A128_3_LA | . | . | . | . | . | . | 1 | . | . | . | 1 | . | . | . | . | . | . |
| AD353T1_A129_3_LA | . | . | . | . | . | . | 1 | . | . | . | 1 | . | . | . | . | . | . |
| AD355T2_A174_4_LA | . | . | . | . | . | . | ? | . | . | . | . | . | 1 | . | . | . | . |
| AD356T1_A175_4_LA | . | . | . | . | . | . | 1 | . | . | . | 1 | . | . | . | . | . | . |
| AD360T1_A176_4_LA | . | . | . | 1 | . | . | . | . | . | . | . | 1 | . | . | . | . | . |
| AD375T2_A286_10_LA | . | . | . | . | . | . | 1 | . | . | . | 1 | . | . | . | . | . | . |
| AD361T1_A177_4_LA | . | . | . | . | . | . | ? | . | . | . | . | . | 1 | . | . | . | . |
| AD362T1_A282_10_LA | . | . | . | . | . | . | 1 | . | . | . | 1 | . | . | . | . | . | . |
| AD363T1_A283_10_LA | . | . | . | . | . | . | 1 | . | . | . | 1 | . | . | . | . | . | . |
| AD366T1_A109_3_LA | . | . | . | . | . | . | 1 | . | . | . | 1 | . | . | . | . | . | . |
| AD367T1_A110_3_LA | . | . | . | . | . | . | 1 | . | . | . | 1 | . | . | . | . | . | . |
| AD368T2_A285_10_LA | . | . | . | . | . | ? | 1 | . | . | . | 1 | . | . | . | . | . | . |
| AD370T1_A112_3_LA | . | . | . | . | . | . | 1 | . | . | . | 1 | . | . | . | . | . | . |
| AD374T1_A114_3_LA | . | . | . | . | . | . | 1 | . | . | . | 1 | . | . | . | . | . | . |
| AD375T1_A115_3_LA | . | . | . | . | . | . | 1 | . | . | . | 1 | . | . | . | . | . | . |
| AD379T2_A287_10_LA | . | . | . | . | . | . | 1 | . | . | . | 1 | . | . | . | . | . | . |
| AD379T1_A116_3_LA | . | . | . | . | . | . | 1 | . | . | . | 1 | . | . | . | . | . | . |
| AD382T3_A225_8_LA | . | ? | . | . | . | . | 1 | . | . | . | 1 | . | . | . | . | . | . |
| AD382T1_A117_3_LA | . | . | . | . | . | . | 1 | . | . | . | 1 | . | . | . | . | . | . |
| AD383T2_A119_3_LA | . | . | . | . | . | . | 1 | . | . | . | 1 | . | . | . | . | . | . |
| AD383T1_A118_3_LA | . | . | . | . | . | . | 1 | . | . | . | 1 | . | . | . | . | . | . |
| ADA4T1_A386_7_LA | . | . | . | . | . | . | 1 | . | . | . | 1 | . | . | . | . | . | . |
| SQ10T1_A362_6_LS | . | . | . | . | . | . | . | . | . | . | . | . | 1 | . | . | . | . |
| SQ1174_A317_5_LS | . | . | . | . | . | . | 1 | . | . | . | 1 | . | . | . | . | . | . |
| SQ13T1_A364_6_LS | . | . | . | . | . | . | 1 | . | . | . | 1 | | . | . | . | . | . |
| SQ14T1_A365_6_LS | . | . | . | . | . | . | 1 | . | . | . | 1 | . | . | . | . | . | . |
| SQ1670_A318_5_LS | . | . | . | . | . | . | 1 | . | . | . | 1 | . | . | . | . | . | . |
| SQ20T1_A366_6_LS | . | . | . | . | . | . | 1 | . | . | . | 1 | . | . | . | . | . | . |
| SQ2557_A320_5_LS | . | . | . | . | . | . | ? | . | . | . | . | . | 1 | . | . | . | . |
| SQ2572_A321_5_LS | . | ? | . | . | . | . | 1 | . | . | . | 1 | . | . | . | . | . | . |
| SQ2921_A322_5_LS | . | . | . | . | . | . | 1 | . | . | . | 1 | . | . | . | . | . | . |
| SQ3197_A323_5_LS | . | . | . | . | . | . | 1 | . | . | . | 1 | . | . | . | . | . | . |
| SQ3529_A324_5_LS | . | . | . | . | . | . | 1 | . | . | . | 1 | . | . | . | . | . | . |
| SQ3624_A325_5_LS | . | . | . | . | . | . | 1 | . | . | . | 1 | . | . | . | . | . | . |
| SQ4172_A326_5_LS | . | . | . | . | . | . | 1 | . | . | . | 1 | . | . | . | . | . | . |
| SQ4389_A327_5_LS | . | . | . | . | . | . | 1 | . | . | . | 1 | . | . | . | . | . | . |
| SQ4T1_A358_6_LS | . | . | . | . | . | . | 1 | . | . | . | 1 | . | . | . | . | . | . |
| SQ5897_A328_5_LS | . | . | . | . | . | . | . | . | . | . | . | . | 1 | . | . | . | . |
| SQ5T1_A359_6_LS | . | . | . | . | . | . | . | . | . | . | . | . | 1 | . | . | . | . |
| SQ6147_A329_5_LS | . | . | . | . | . | . | 1 | . | . | . | 1 | . | . | . | . | . | . |
| SQ6T1_A360_6_LS | . | . | . | . | . | . | 1 | . | . | . | 1 | . | . | . | . | . | . |
| SQ7324_A416_7_LS | . | . | . | . | . | . | 1 | . | . | . | 1 | . | . | . | . | . | . |
| SQ8T1_A361_6_LS | . | . | . | . | . | . | 1 | . | . | . | 1 | . | . | . | . | . | . |

| | | | | | | | |
|---|---|---|---|---|---|---|---|
| SUMMARY | 155 | 1 | 41 | 197 | 79 | 1 | 21 |

Table 16 summarizes the results of the Bhattachatjee et al. cross validation of the Su-Hampton 2001 model by tissue type. In Table 16, #Samples is the number of biological specimens 58 tested, #COR is the number of samples correctly identified, #INCOR is the number of incorrectly identified samples, #IND is the number of indeterminates.

TABLE 16
Bhattacharjee et al. cross validation of the
Su-Hampton 2001 model by tissue type

| Abbr | Origin Site | #Samples | #COR | #INCOR | #IND |
|---|---|---|---|---|---|
| CO | Colorectal | 14 | 5 | 1 | 8 |
| LU | Lung | 197 | 155 | 1 | 41 |
| | TOTALS | 211 | 160 | 2 | 49 |

Table 17 shows the percentage of samples correctly identified, incorrectly identified, and the number of samples for which the biological classification was indeterminate.

TABLE 17
Bhattacharjee et al. Model percentage summary

| Abbr | Origin Site | % Correct | % Incorrect | % Indeterminate |
|---|---|---|---|---|
| CO | Colorectal | 35 | 7 | 57 |
| LU | Lung | 78 | 0 | 20 |
| | OVERALL | 76 | 1 | 23 |

The Su-Hampton 2001 model was able to correctly classify 78% (155/197) of the samples as lung carcinoma. Interestingly, the Su-Hampton 2001 model also correctly classified 5 of 14 samples as most likely representing colorectal carcinomas. By including the colorectal samples, Su-Hampton 2001 model correctly classified 76% (160/211) of the samples from Bhattacharjee et al. The model also declared as indeterminate 23 percent of the samples (49 samples) indicating that such samples could not be classified with confidence.

### 6.3 Cancer of Unknown Primary/Alternative Embodiment

Carcinoma of Unknown Primary is diagnosed when the primary site where the cancer originated cannot be determined. Standard pathological techniques identify the primary in only 25% of these cases. See, for example, Hainsworth et al., 1993, New England Journal of Medicine, 329, 257-263; and Raber et al., 1992, Curr Opin Oncol. 4, pp. 3-9. An even larger number of patients present with tumors of uncertain primary that can be a recurrence of an earlier, successfully treated disease. Knowing the primary site has clinical importance for optimal cancer management and improves prognosis. See, for example, Buckhaults et al., 2003, Cancer Res. 63, 4144-9; and Abbruzzese et al., 1995, J Clin Oncol., 13, 2094-103.

Determining the anatomical site of origin is presently fundamental for selecting the optimal treatment of patients with cancer. Currently there are no definitive, cost-effective analytical methods to identify the site of origin in carcinoma when the primary is unknown or uncertain. This study was undertaken to demonstrate that when applied to microarray gene expression data, models developed in accordance with Section 5.9 convert gene expression profiles into actionable reports that identify the site of origin for tumors of unknown or uncertain origin.

Steps 602-616.

Published data from a variety of sources was used. The validation data comprised output files from microarray (Affymetrix U95A) processing of 148 frozen tumor tissue samples. Each specimen was from a primary or metastatic lesion from one of five known sites (prostate, breast, colorectum, lung, ovary). All data was analyzed in accordance with the techniques describe in Section 5.9.

To make models of prostate, breast, colorectum, lung, and ovary cancer, cellular constituents identified in Su et al. were considered (FIG. 6, step 610). Such cellular constituents were ranked using mutual information (FIG. 6, step 612). Cellular constituents that were highly ranked on the basis of mutual information were selected for use in ratios (FIG. 6, step 616). Each ratio consisted of a select cellular constituent in the numerator and a select cellular constituent in the denominator as set forth in Table 18.

TABLE 18
The Su-Hampton 5.2 models developed using
methods described in Section 5.9.

| | | Numerator | Denominator | | |
|---|---|---|---|---|---|
| | | (Affymetrix | (Affymetrix | | |
| | Tissue | accession | accession | Negative | Positive |
| Version | name | ID) | ID) | Threshold | Threshold |
| 5.2 | Breast | 33878_at | 328383_at | 1 | 2.5 |
| 5.2 | Breast | 36329_at | 38739_at | 0.2 | 3 |
| 5.2 | Breast | 40046_r_at | 32563_at | 0.05 | 0.2 |
| 5.2 | Breast | 41348_at | 36685_at | 0.05 | 0.3 |
| 5.2 | Colorectal | 37423_at | 32091_at | 1 | 1.5 |
| 5.2 | Colorectal | 1582_at | 36668_at | 1 | 1.5 |
| 5.2 | Colorectal | 169_at | 39253_2_at | 0 | 0.5 |
| 5.2 | Colorectal | 40736_at | 36571_at | 0.1 | 0.5 |
| 5.2 | Colorectal | 32972_at | 32091_at | 0.3 | 1 |
| 5.2 | Colorectal | 41073_at | 40957_at | 0.1 | 1 |
| 5.2 | Lung | 40928_at | 35778_at | 5 | 14 |
| 5.2 | Lung | 37402_at | 38762_at | 0.5 | 2 |
| 5.2 | Lung | 37351_at | 40162_a_at | 2 | 10 |
| 5.2 | Lung | 35132_at | 37175_at | 0.5 | 50 |
| 5.2 | Lung | 33956_at | 36628_at | 0.2 | 0.7 |
| 5.2 | Lung | 33754_at | 31791_at | 0 | 1 |
| 5.2 | Lung | 33529_at | 35332_at | −1 | 0.9 |
| 5.2 | Ovary | 1500_at | 37148_at | 10 | 40 |
| 5.2 | Ovary | 40401_at | 251_at | 5 | 15 |
| 5.2 | Ovary | 40763_at | 1582_at | 1 | 12 |
| 5.2 | Ovary | 34194_at | 1729_at | 1 | 25 |
| 5.2 | Ovary | 32838_at | 36668_at | 0.1 | 0.5 |
| 5.2 | Ovary | 35277_at | 41468_at | 5 | 45 |
| 5.2 | Prostate | 40794_at | 41827_f_at | 1.1 | 3 |
| 5.2 | Prostate | 41721_at | 38894_g_at | 10 | 70 |
| 5.2 | Prostate | 41468_at | 39649_at | 2 | 10 |
| 5.2 | Prostate | 32200_at | 927_s_at | 0.1 | 5 |
| 5.2 | Prostate | 41172_at | 34778_at | 6 | 14 |

Steps 618-620.

Once the Su-Hampton 5.2 ratios had been constructed for breast cancer, colorectum cancer, lung cancer, cancer of the ovaries, and prostate cancer, threshold values were identified for each of the ratios in each of the models using the methods describe in Section 5.9, above. See also, FIG. 6, step 618. In particular, an ROC curve was generated for each ratio in a model. The points in the convex hull of each ROC curve were selected as candidate threshold values. All possible combinations of the candidate threshold values were tested against the target goal function described in Section 5.9. The combination of candidate threshold values that maximized the goal function were selected as the positive and negative threshold values for the model. This process was repeated for each of the models listed in Table 18 (FIG. 6, step 620).

Step 622.

Final models were tested against a validation data set partition. The results showed that the models developed in accordance with Section 5.9 were accurate. The models identified the correct cancer in 89% of the samples, incorrectly classified 3% of the samples, and provided an indeterminate measurement on 8% of the samples. Table 19 compares the percent correct, incorrect, and indeterminate for the Su-Hampton 5.2 models of Table 18 versus the percent correct, incorrect, and indeterminate for the corresponding models originally published in Su et al. 2001, Cancer Research 61, p. 7388. To generate the data in Table 19, the site of origin of a plurality of tumors was tested using two different model suites. The first model suite consisted of the breast, colorectal, lung, ovary, and prostate models listed in Table 18. The second model suite consisted of the original breast, colorectal, lung, ovary, and prostate models published in Su et al. Each tumor was tested against each model in each of the two model suites.

TABLE 19
Summary of classification results for Su-Hampton 5.2 models
(1) of Table 18 versus Su et al. (2) data based on tissue
type versus the Model Summary

| source | Origin Site | #Samples | #COR | #INDE | #INCOR |
|---|---|---|---|---|---|
| (1) | Breast | 38 | 28 | 7 | 3 |
| (2) | Breast | 14 | 10 | 4 | 0 |
| (1) | Colorectal | 13 | 12 | 0 | 1 |
| (2) | Colorectal | 12 | 11 | 1 | 0 |
| (1) | Lung | 71 | 67 | 4 | 0 |
| (2) | Lung | 10 | 9 | 1 | 0 |
| (1) | Ovary | 9 | 8 | 0 | 1 |
| (2) | Ovary | 18 | 17 | 1 | 0 |
| (1) | Prostate | 17 | 16 | 1 | 0 |
| (2) | Prostate | 16 | 16 | 0 | 0 |

(1) = Su-Hampton 5.2 suite of Table 18;
(2) = Suite reported in Su et al.

In Table 19, #COR stands for the number of correct assignments. A suite scored correctly if (i) exactly one test in the suite (The Su-Hampton 5.2 suite of Table 18 or the suite reported in Su et al.) scored greater than zero and this test corresponded to the actual site of origin, or (ii) exactly two tests in the suite came out positive and one of them corresponded to the correct “tissue source” (e.g., lung for lung cancer) and the other to the “site of origin.”

In Table 19, #INCOR stands for the number of incorrect assignments. A suite scored incorrectly if it either “missassigned” a specimen or was designated a “missed metastasis.” A suite “misassigned” a specimen when exactly one test in the suite scored greater than zero and this test corresponded to a tissue type other than the “site of origin” or the “tissue source”. A suite also “misassigned” a specimen when exactly two tests in the suite scored greater than zero and one of them corresponded to the “tissue source” and the other corresponded to a site other than the “site of origin”. A suite wad designated a “missed metastasis” if exactly one test in the suite scored greater than zero and this test corresponded to the “tissue source” but not to the “site of origin”.

In Table 19, #INDE stands for the number of indeterminate assignments. A suite was indeterminate if exactly zero tests in the suite scored greater than zero. A suite was also indeterminate if exactly two tests in the suite scored greater than zero and none of them corresponded to the tissue source. A suite was also indeterminate if more than two tests in the suite scored greater than zero.

FIG. 9 compares the results of the present example to that of other labs. As illustrated in FIG. 9, the models developed using the methods disclosed in Section 5.9 produce more accurate results than previously identified.

### 7. REFERENCES CITED

All references and databases cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a computer readable storage medium. For instance, the computer program product could contain the program modules shown in FIG. 1. These program modules may be stored on a CD-ROM, magnetic disk storage product, or any other computer readable data or program storage product. The software modules in the computer program product can also be distributed electronically, via the Internet or otherwise, by transmission of a computer data signal (in which the software modules are embedded) on a carrier wave.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A computer program product for use in conjunction with a computer system, the computer program product comprising a computer readable storage medium and a computer program mechanism embedded therein, the computer program mechanism comprising:
a model characterized by a model score, the model comprising a plurality of tests, wherein
each respective test in said plurality of tests is characterized by a test value that is determined by a function of the characteristics of one or more cellular constituents in a plurality of cellular constituents in a test organism of a species or a test biological specimen from an organism of said species; and
each respective test in the plurality of tests is independently assigned a positive threshold and a negative threshold wherein
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold.

2. The computer program product of claim 1 wherein the plurality of tests consists of two or more tests.

3. The computer program product of claim 1 wherein the plurality of tests consists of five or more tests.

4. The computer program product of claim 1 wherein the plurality of tests consists of between two and fifty tests.

5. The computer program product of claim 1 wherein each said function of a test in the plurality of tests uses a characteristic of a predetermined cellular constituent.

6. The computer program product of claim 1 wherein each said function uses a ratio between a numerator and a denominator, wherein the numerator comprises a characteristic of a predetermined first cellular constituent in the test organism or test biological specimen and the denominator comprises a characteristic of a predetermined second cellular constituent in the test organism or test biological specimen.

7. The computer program product of claim 1 wherein said model represents the absence or presence of a biological feature in the test organism or the test biological specimen, wherein
the test organism or the test biological specimen is deemed to have the biological feature when the model score is positive; and
the test organism or the test biological specimen is deemed not to have the biological feature when the model score is negative.

8. The computer program product of claim 7 wherein said biological feature is a disease.

9. The computer program product of claim 8 wherein said disease is cancer.

10. The computer program product of claim 8 wherein said disease is breast cancer, lung cancer, prostate cancer, colorectal cancer, ovarian cancer, bladder cancer, gastric cancer, or rectal cancer.

11. The computer program product of claim 7 wherein
each said function uses a ratio between a numerator and a denominator, wherein the numerator comprises a characteristic of a predetermined first cellular constituent in the test organism or test biological specimen and the denominator comprises a characteristic of a predetermined second cellular constituent in the test organism or test biological specimen;
the first cellular constituent is more abundant in members of said species or biological specimens that have said biological feature than in members of said species or biological specimens that do not have said biological feature; and
the second cellular constituent is less abundant in members of said species or biological specimens that have said biological feature than in members of said species or biological specimens that do not have said biological feature.

12. The computer program product of claim 1 wherein the plurality of tests comprises a first test and a second test and the identities of the one or more cellular constituents whose characteristics in the test organism or test biological specimen used to determine the value of the first test are different than the identities of the one or more cellular constituents whose characteristics in the test organism or test biological specimen used to determine the value of the second test.

13. The computer program product of claim 1 wherein the plurality of tests comprises a first test and a second test and an identity of a cellular constituent in the one or more cellular constituents whose characteristics are used to determine the value of the first test is the same as the identity of a cellular constituent in the one or more cellular constituents whose characteristics are used to determine the value of the second test.

14. The computer program product of claim 1 wherein a test in the plurality of tests contributes
a single positive unit to the model score when the test value for the test exceeds the positive threshold assigned to the test;
zero units to the model score when the test value for the test is less than the positive threshold assigned to the test and greater than the negative threshold assigned to the test; and
a single negative unit to the model score when the test value for the test is less than the negative threshold assigned to the test.

15. The computer program product of claim 1 wherein a test in the plurality of tests contributes
a weighted positive unit to the model score when the test value for the test exceeds the positive threshold assigned to the test;
zero units to the model score when the test value for the test is less than the positive threshold assigned to the test and greater than the negative threshold assigned to the test; and
a weighted negative unit to the model score when the test value for the test is less than the negative threshold assigned to the test.

16. The computer program product of claim 15 wherein the magnitude of the weighted positive unit is determined by an amount the test value exceeds the positive threshold assigned to the test.

17. The computer program product of claim 15 wherein the magnitude of the weighted positive unit and the weighted negative unit is determined by a degree of confidence in the test.

18. The computer program product of claim 17 wherein the magnitude of the weighted positive unit and the weighted negative unit is determined by an area under a receiver operating characteristic (ROC) curve used to assign the positive threshold and the negative threshold to the test.

19. The computer program product of claim 15 wherein the magnitude of the weighted negative unit is determined by an amount the test value is less than the negative threshold assigned to the test.

20. The computer program product of claim 1 wherein the species is human.

21. The computer program product of claim 1 wherein the test biological specimen is a biopsy or other form of sample from a tumor, blood, bone, a breast, a lung, a prostate, a colorectum, an ovary, a bladder, a stomach, or a rectum.

22. The computer program product of claim 1, the computer program product further comprising
a cellular constituent data set; and
instructions for using the cellular constituent data set to assign a positive threshold and a negative threshold to a test in said plurality of tests.

23. The computer program product of claim 22 wherein the cellular constituent data set comprises:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism.

24. The computer program product of claim 23 wherein the plurality of cellular constituent charactistic measurements comprises between 5 and 1000 cellular constituent characteristic measurements.

25. The computer program product of claim 23 wherein the plurality of cellular constituent characteristic measurements comprises more than 50 cellular constituent characteristic measurements.

26. The computer program product of claim 23 wherein the plurality of cellular constituent characteristic measurements comprises more than 1000 cellular constituent characteristic measurements.

27. The computer program product of claim 22 wherein said instructions for using the cellular constituent data set to assign a positive threshold and a negative threshold to a test in said plurality of tests comprises selecting:
a first subset of said plurality of cellular constituents, wherein each cellular constituent in said first subset of cellular constituents is up-regulated in organisms in which said biological feature is present; and
a second subset of said plurality of cellular constituents, wherein each cellular constituent in said second subset of cellular constituents is down-regulated in organisms in which said biological feature is present.

28. The computer program product of claim 27, wherein said instructions for using the cellular constituent data set to assign a positive threshold and a negative threshold to a test in said plurality of tests comprises
constructing a test in said plurality of tests, wherein the function of the test is a ratio between (i) a characteristic of a cellular constituent in said first subset and (ii) a characteristic of a cellular constituent in said second subset.

29. The computer program product of claim 1 wherein a cellular constituent in said plurality of cellular constituents is mRNA, cRNA or cDNA.

30. The computer program product of claim 1 wherein a cellular constituent in said one or more cellular constituents is a nucleic acid or a ribonucleic acid and the characteristic of said cellular constituent is obtained by measuring a transcriptional state of all or a portion of said cellular constituent in said test organism or said test biological specimen.

31. The computer program product of claim 1 wherein a cellular constituent in said one or more cellular constituents is a protein and the characteristic of said cellular constituent is obtained by measuring a translational state of said cellular constituent in said test organism or said test biological specimen.

32. The computer program product of claim 1 wherein the characteristic of a cellular constituent in the one or more cellular constituents is determined using isotope-coded affinity tagging followed by tandem mass spectrometry analysis of the cellular constituent using a sample obtained from the test organism or the test biological specimen.

33. The computer program product of claim 1 wherein the characteristic of a cellular constituent in said one or more constituents is determined by measuring an activity or a post-translational modification of the cellular constituent in a sample obtained from the test organism or in the test biological specimen.

34. A computer comprising:
a central processing unit;
a memory, coupled to the central processing unit, the memory storing:
a model characterized by a model score, the model comprising a plurality of tests, wherein
each respective test in said plurality of tests is characterized by a test value that is determined by a function of the characteristics of one or more cellular constituents in a plurality of cellular constituents in a test organism of a species or a test biological specimen from an organism of said species; and
each respective test in the plurality of tests is independently assigned a positive threshold and a negative threshold wherein
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold.

35. A computer program product for use in conjunction with a computer system, the computer program product comprising a computer readable storage medium and a computer program mechanism embedded therein, the computer program product comprising:
(A) instructions for computing a mutual information score I(X,Y) between X and Y wherein
X is a variable wherein each value x of X represents a presence or an absence of a biological feature in a member of all or a portion of a population of a species, wherein said population includes members that have said biological feature and members that do not have said biological feature;
Y is a variable wherein each value y of Y represents a characteristic of a cellular constituent measured in a biological specimen from a member of all or said portion of said population of said species; and
(B) instructions for repeating said instructions (A) for one or more cellular constituents in a plurality of cellular constituents thereby identifying a cellular constituent having the property that the mutual information between the variable Y associated with the cellular constituent and X is larger than the respective mutual information between (i) the respective variable Y associated with each cellular constituent in one or more other cellular constituents in said plurality of cellular constituents and (ii) X.

36. The computer program product of claim 35, wherein the computer program product further comprises:
instructions for accessing one or more data structures collectively comprising a cellular constituent characteristic of each cellular constituent in said plurality of cellular constituents measured in a biological specimen from each member of said population of said species;
instructions for dividing the one or more data structures into a training data set partition and a test data set partition wherein
said training data set partition comprises cellular constituent characteristics of said plurality of cellular constituents measured in biological specimens from a randomly selected first subset of said population; and
said test data set partition comprises cellular constituent characteristics of said plurality of cellular constituents measured in biological specimens from a randomly selected second subset of said population, provided that biological specimens represented by said second subset are not represented by said first subset; and wherein
each value x of X represents a presence or an absence of a biological feature in a member of said training data set partition;
each value y of Y represents a characteristic of a cellular constituent measured in a biological specimen from said training data set partition.

37. The computer program product of claim 35, wherein
$$I ( X , Y ) = H ( X ) - H ( X \vert Y ) = {\sum }_{x , y} r ( x , y ) {log}_{2} \frac{r ( x , y )}{x y}$$
wherein,
H(X) is the entropy of X;
H(X|Y) is the entropy of X given Y; and
r(x,y) is the joint distribution of X and Y.

38. The computer program product of claim 35 wherein said biological feature is a disease.

39. The computer program product of claim 38 wherein said disease is cancer.

40. The computer program product of claim 38 wherein said disease is breast cancer, lung cancer, prostate cancer, colorectal cancer, ovarian cancer, bladder cancer, gastric cancer, or rectal cancer.

41. The computer program product of claim 35 wherein the species is human.

42. The computer program product of claim 35 wherein the biological specimen from a member of the population of the species is a biopsy or other form of sample from a tumor, blood, bone, a breast, a lung, a prostate, a colorectum, an ovary, a bladder, a stomach, or a rectum.

43. The computer program product of claim 35 wherein a cellular constituent in said plurality of cellular constituents is mRNA, cRNA or cDNA.

44. The computer program product of claim 35 wherein a cellular constituent in said one or more cellular constituents is a nucleic acid or a ribonucleic acid and the characteristic of said cellular constituent in a biological specimen from a member of the population is obtained by measuring a transcriptional state of all or a portion of said cellular constituent in said biological specimen.

45. The computer program product of claim 35 wherein a cellular constituent in said one or more cellular constituents is a protein and the characteristic of said cellular constituent in a biological specimen from a member of the population is obtained by measuring a translational state of said cellular constituent in said biological specimen.

46. The computer program product of claim 35 wherein the characteristic of a cellular constituent in said one or more cellular constituents in a biological specimen from a member of the population is determined using isotope-coded affinity tagging followed by tandem mass spectrometry analysis of the cellular constituent using the biological specimen.

47. The computer program product of claim 35 wherein the characteristic of a cellular constituent in said one or more cellular constituents in a biological specimen from a member of the population is determined by measuring an activity or a post-translational modification of the cellular constituent in the biological specimen.

48. The computer program product of claim 36 wherein said first subset of said population comprises between ten and one thousand members.

49. The computer program product of claim 36 wherein said first subset of said population comprises more than 100 members.

50. The computer program product of claim 36 wherein said second subset of said population comprises between ten and one thousand members.

51. The computer program product of claim 36 wherein said second subset of said population comprises more than 100 members.

52. The computer program product of claim 35 wherein said instructions for repeating are executed more than eight times for more than eight different cellular constituents in said plurality of cellular constituents.

53. The computer program product of claim 35 wherein said instructions for repeating are executed more than twenty times for more than twenty different cellular constituents in said plurality of cellular constituents.

54. The computer program product of claim 35 wherein said instructions for repeating are executed between ten and ten thousand times for between ten and ten thousand different cellular constituents in said plurality of cellular constituents.

55. The computer program product of claim 35, wherein the computer program product further comprises:
instructions for ranking a plurality of cellular constituents tested by instances of said instructions for computing (A) by the respective mutual information scores of the one or more cellular constituents computed by said instructions for computing (A) in order to form a ranked list of cellular constituents; and
instructions for selecting a plurality of cellular constituents from a top-ranked portion of the ranked list of cellular constituents for inclusion in a model that is diagnostic of said biological feature.

56. The computer program product of claim 55 wherein said top-ranked portion of the ranked list of cellular constituent is the first five cellular constituents in the ranked list.

57. The computer program product of claim 55 wherein said top-ranked portion of the ranked list of cellular constituent is the first ten cellular constituents in the ranked list.

58. The computer program product of claim 55 wherein said top-ranked portion of the ranked list of cellular constituent is the first twenty cellular constituents in the ranked list.

59. The computer program product of claim 55 wherein said top-ranked portion of the ranked list of cellular constituent is the first one hundred cellular constituents in the ranked list.

60. The computer program product of claim 55 wherein said top-ranked portion of the ranked list of cellular constituent is the upper one percent of the cellular constituents in the ranked list.

61. The computer program product of claim 55 wherein said top-ranked portion of the ranked list of cellular constituent is the upper three percent of the cellular constituents in the ranked list.

62. The computer program product of claim 55 wherein said top-ranked portion of the ranked list of cellular constituent is the upper ten percent of the cellular constituents in the ranked list.

63. The computer program product of claim 55 wherein said instructions for selecting cellular constituents comprises:
instructions for dividing said top-ranked portion of the ranked list into a first category and a second category wherein
cellular constituents in said first category are those cellular constituents whose characteristic values in all or said portion of said population positively correlate with X; and
cellular constituents in said second category are those cellular constituents whose characteristic values in all or said portion of said population negatively correlate with X.

64. The computer program product of claim 63 wherein said instructions for selecting cellular constituents further comprises:
instructions for constructing said model, wherein said model comprises a plurality of tests and wherein each test includes a first cellular constituent in said first category and a second cellular constituent in said second category.

65. The computer program product of claim 64 wherein the first cellular constituent in each test in said model is different.

66. The computer program product of claim 64 wherein the second cellular constituent in each test in said model is different.

67. The computer program product of claim 64 wherein said model is characterized by a model score and wherein each respective test in said plurality of tests is characterized by a test value that is determined by a function of the characteristic of the first cellular constituent and the characteristic of the second cellular constituent in a test biological specimen from an organism.

68. The computer program product of claim 67 wherein
the function of a test in said plurality of tests is a ratio in which the characteristic of the first cellular constituent is the numerator of the ratio and the characteristic of the second cellular constituent is the denominator of the ratio;
the test positively contributes to the model score when the ratio exceeds the positive threshold;
the test does not contribute to the model score when the ratio is less than the positive threshold and greater than the negative threshold; and
the test negatively contributes to the model score when the ratio is less than the negative threshold.

69. The computer program product of claim 67 wherein
each respective test in the plurality of tests is independently assigned a positive threshold and a negative threshold wherein
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold.

70. The computer program product of claim 64 wherein the plurality of tests consists of two or more tests.

71. The computer program product of claim 64 wherein the plurality of tests consists of five or more tests.

72. The computer program product of claim 64 wherein the plurality of tests consists of between two and fifty tests.

73. The computer program product of claim 67 wherein said model represents the absence or presence of a biological feature in the test biological specimen, wherein
the test biological specimen is deemed to have the biological feature when the model score is positive; and
the test biological specimen is deemed to not have the biological feature when the model score is negative.

74. The computer program product of 69, wherein said computer program product further comprises instructions for validating said model by quantifying the specificity or the sensitivity of the model against the cellular constituent characteristic data of a portion of the population of the species not used to assign a positive threshold or a negative threshold to a test in the plurality of tests in the model.

75. A first computer comprising:
a central processing unit;
a memory, coupled to the central processing unit, the memory storing:
(A) instructions for computing a mutual information score I(X,Y) between X and Y wherein
X is a variable wherein each value x of X represents a presence or an absence of a biological feature in a member of all or a portion of a population of a species, wherein said population includes members that have said biological feature and members that do not have said biological feature; and
Y is a variable wherein each value y of Y represents a characteristic of a cellular constituent measured in a biological specimen from a member of all or said portion of said population of said species; and
(B) instructions for repeating said instructions (A) for one or more cellular constituents in a plurality of cellular constituents thereby identifying a cellular constituent having the property that the mutual information between the variable Y associated with the cellular constituent and X is larger than the respective mutual information between (i) the respective variable Y associated with each cellular constituent in one or more other cellular constituents in said plurality of cellular constituents and (ii) X.

76. The first computer of claim 75 wherein the memory further stores
instructions for accessing one or more data structures collectively comprising a cellular constituent characteristic of each cellular constituent in said plurality of cellular constituents measured in a biological specimen from each member of said population of said species; and
instructions for dividing the one or more data structures into a training data set partition and a test data set partition wherein
said training data set partition comprises cellular constituent characteristics of said plurality of cellular constituents measured in biological specimens from a randomly selected first subset of said population; and
said test data set partition comprises cellular constituent characteristics of said plurality of cellular constituents measured in biological specimens from a randomly selected second subset of said population, provided that biological specimens represented by said second subset are not represented by said first subset; and wherein
each value x of X represents a presence or an absence of a biological feature in a member of said training data set partition;
each value y of Y represents a characteristic of a cellular constituent measured in a biological specimen from said training data set partition

77. The first computer of claim 76 wherein the one or more data structures are in the memories of one or more second computers, wherein each of the one or more second computers are addressable by said first computer across one or more network connections.

78. The first computer of claim 76 the one or more data structures are in said memory.

79. A method comprising:
computing a mutual information score I(X,Y) between X and Y wherein
X is a variable wherein each value x of X represents a presence or an absence of a biological feature in a member of all or a portion of a population of a species, wherein said population includes members that have said biological feature and members that do not have said biological feature;
Y is a variable wherein each value y of Y represents a characteristic of a cellular constituent measured in a biological specimen from a member of all or said portion of said population of said species; and
repeating said computing for one or more cellular constituents in a plurality of cellular constituents thereby identifying a cellular constituent having the property that the mutual information between the variable Y associated with the cellular constituent and X is larger than the respective mutual information between (i) the respective variable Y associated with each cellular constituent in one or more other cellular constituents in said plurality of cellular constituents and (ii) X.

80. The method of claim 79, the method further comprising:
accessing one or more data structures collectively comprising a cellular constituent characteristic of each cellular constituent in said plurality of cellular constituents measured in a biological specimen from each member of said population of said species;
dividing the one or more data structures into a training data set partition and a test data set partition wherein
said training data set partition comprises cellular constituent characteristics of said plurality of cellular constituents measured in biological specimens from a randomly selected first subset of said population; and
said test data set partition comprises cellular constituent characteristics of said plurality of cellular constituents measured in biological specimens from a randomly selected second subset of said population, provided that biological specimens represented by said second subset are not represented by said first subset; and wherein
each value x of X represents a presence or an absence of a biological feature in a member of said training data set partition;
each value y of Y represents a characteristic of a cellular constituent measured in a biological specimen from said training data set partition.

81. The method of claim 79, wherein
$$I ( X , Y ) = H ( X ) - H ( X \vert Y ) = {\sum }_{x , y} r ( x , y ) {log}_{2} \frac{r ( x , y )}{x y}$$
wherein,
H(X) is the entropy of X;
H(X|Y) is the entropy of X given Y; and
r(x,y) is the joint distribution of X and Y.

82. The method of claim 79 wherein said biological feature is a disease.

83. The method of claim 82 wherein said disease is cancer.

84. The method of claim 82 wherein said disease is breast cancer, lung cancer, prostate cancer, colorectal cancer, ovarian cancer, bladder cancer, gastric cancer, or rectal cancer.

85. The method of claim 79 wherein the species is human.

86. The method of claim 79 wherein the biological specimen from a member of the population of the species is a biopsy or other form of sample from a tumor, blood, bone, a breast, a lung, a prostate, a colorectum, an ovary, a bladder, a stomach, or a rectum.

87. The method of claim 79 wherein a cellular constituent in said plurality of cellular constituents is mRNA, cRNA or cDNA.

88. The method of claim 79 wherein a cellular constituent in said one or more cellular constituents is a nucleic acid or a ribonucleic acid and the characteristic of said cellular constituent in a biological specimen from a member of the population is obtained by measuring a transcriptional state of all or a portion of said cellular constituent in said biological specimen.

89. The method of claim 79 wherein a cellular constituent in said one or more cellular constituents is a protein and the characteristic of said cellular constituent in a biological specimen from a member of the population is obtained by measuring a translational state of said cellular constituent in said biological specimen.

90. The method of claim 79 wherein the characteristic of a cellular constituent in said one or more cellular constituents in a biological specimen from a member of the population is determined using isotope-coded affinity tagging followed by tandem mass spectrometry analysis of the cellular constituent using the biological specimen.

91. The method of claim 79 wherein the characteristic of a cellular constituent in said one or more cellular constituents in a biological specimen from a member of the population is determined by measuring an activity or a post-translational modification of the cellular constituent in the biological specimen.

92. The method of claim 80 wherein said first subset of said population comprises between ten and one thousand members.

93. The method of claim 80 wherein said first subset of said population comprises more than 100 members.

94. The method of claim 80 wherein said second subset of said population comprises between ten and one thousand members.

95. The method of claim 80 wherein said second subset of said population comprises more than 100 members.

96. The method of claim 79 wherein said repeating (B) is done more than eight times for more than eight different cellular constituents in said plurality of cellular constituents.

97. The method of claim 79 wherein said repeating (B) is done more than twenty times for more than twenty different cellular constituents in said plurality of cellular constituents.

98. The method of claim 79 wherein said repeating (B) is done between ten and ten thousand times for between ten and ten thousand different cellular constituents in said plurality of cellular constituents.

99. The method of claim 79, the method further comprising:
ranking a plurality of cellular constituents tested by instances of said computing (B) by the respective mutual information scores of the one or more cellular constituents computed by said computing (B) in order to form a ranked list of cellular constituents; and
selecting a plurality of cellular constituents from a top-ranked portion of the ranked list of cellular constituents for inclusion in a model that is diagnostic of said biological feature.

100. The method of claim 99 wherein said top-ranked portion of the ranked list of cellular constituent is the first five cellular constituents in the ranked list.

101. The method of claim 99 wherein said top-ranked portion of the ranked list of cellular constituent is the first ten cellular constituents in the ranked list.

102. The method of claim 99 wherein said top-ranked portion of the ranked list of cellular constituent is the first twenty cellular constituents in the ranked list.

103. The method of claim 99 wherein said top-ranked portion of the ranked list of cellular constituent is the first one hundred cellular constituents in the ranked list.

104. The method of claim 99 wherein said top-ranked portion of the ranked list of cellular constituent is the upper one percent of the cellular constituents in the ranked list.

105. The method of claim 99 wherein said top-ranked portion of the ranked list of cellular constituent is the upper three percent of the cellular constituents in the ranked list.

106. The method of claim 99 wherein said top-ranked portion of the ranked list of cellular constituent is the upper ten percent of the cellular constituents in the ranked list.

107. The method of claim 99 wherein said selecting cellular constituents comprises:
dividing said top-ranked portion of the ranked list into a first category and a second category wherein
cellular constituents in said first category are those cellular constituents whose characteristic values in all or said portion of said population positively correlate with X; and
cellular constituents in said second category are those cellular constituents whose characteristic values in all or said portion of said population negatively correlate with X.

108. The method of claim 107 wherein said selecting cellular constituents further comprises:
constructing said model, wherein said model comprises a plurality of tests and wherein each test in the plurality of tests includes a first cellular constituent in said first category and a second cellular constituent in said second category.

109. The method of claim 108 wherein the first cellular constituent in each test in said model is different.

110. The method of claim 108 wherein the second cellular constituent in each test in said model is different.

111. The method of claim 108 wherein said model is characterized by a model score and wherein
each respective test in said plurality of tests is characterized by a test value that is determined by a function of the characteristic of the first cellular constituent and the characteristic of the second cellular constituent in a test biological specimen from an organism.

112. The method of claim 111 wherein
the function of a test in said plurality of tests is a ratio in which the characteristic of the first cellular constituent is the numerator of the ratio and the characteristic of the second cellular constituent is the denominator of the ratio;
the test positively contributes to the model score when the ratio exceeds a positive threshold;
the test does not contribute to the model score when the ratio is less than the positive threshold and greater than a negative threshold; and
the test negatively contributes to the model score when the ratio is less than the negative threshold.

113. The method of claim 111 wherein
each respective test in the plurality of tests is independently assigned a positive threshold and a negative threshold wherein
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold.

114. The method of claim 108 wherein the plurality of tests consists of two or more tests.

115. The method of claim 108 wherein the plurality of tests consists of five or more tests.

116. The method of claim 108 wherein the plurality of tests consists of between two and fifty tests.

117. The method of claim 111 wherein said model represents the absence or presence of a biological feature in the test biological specimen, wherein
the test biological specimen is deemed to have the biological feature when the model score is positive; and
the test biological specimen is deemed to not have the biological feature when the model score is negative.

118. The method of 113, the method further comprising:
validating said model by quantifying the specificity or the sensitivity of the model against the cellular constituent characteristic data of a portion of the population of the species not used to assign a positive threshold or a negative threshold to a test in the plurality of tests in the model.

119. A computer program product for use in conjunction with a computer system, the computer program product comprising a computer readable storage medium and a computer program mechanism embedded therein, the computer program mechanism comprising:
a model characterized by a model score, the model comprising a plurality of tests, wherein each respective test in said plurality of tests is characterized by a test value that is determined by a function of the characteristic of one or more cellular constituents in a plurality of cellular constituents in a test organism of a species or a test biological specimen from an organism of said species;
instructions for identifying one or more candidate thresholds for each respective test in said plurality of tests; and
instructions for scoring each candidate threshold combination in a plurality of candidate threshold combinations, wherein each candidate threshold combination in said plurality of candidate threshold combinations comprises one or more candidate thresholds for each test in said plurality of tests that was identified by said instructions for identifying.

120. The computer program product of claim 119 wherein said instructions for identifying one or more candidate thresholds for each respective test in said plurality of tests comprises instructions for identifying a positive threshold and a negative threshold for each respective test in said plurality of tests wherein each respective test
positively contributes to the model score when the test value for the respective test exceeds the positive threshold;
does not contribute to the model score when the test value for the respective test is less than the positive threshold and greater than the negative threshold; and
negatively contributes to the model score when the test value for the respective test is less than the negative threshold.

121. The computer program product of claim 120 wherein the function of a test in the plurality of tests comprises a characteristic of a predetermined cellular constituent; wherein
the test positively contributes to the model score when the characteristic of the cellular constituent in the test organism or the test biological specimen exceeds the positive threshold;
the test does not contribute to the model score when the characteristic of the cellular constituent in the test organism or the test biological specimen is less than the positive threshold and greater than the negative threshold; and
the test negatively contributes to the model score when the characteristic of the cellular constituent in the test organism or the test biological specimen is less than the negative threshold.

122. The computer program product of claim 120 wherein the function of a test in the plurality of tests comprises a ratio between a numerator and a denominator, wherein the numerator comprises a characteristic of a predetermined first cellular constituent in the test organism or test biological specimen and the denominator comprises a characteristic of a predetermined second cellular constituent in the test organism or test biological specimen; wherein
the test positively contributes to the model score when the ratio exceeds the positive threshold;
the test does not contribute to the model score when the ratio is less than the positive threshold and greater than the negative threshold; and
the test negatively contributes to the model score when the ratio is less than the negative threshold.

123. The computer program product of claim 119 wherein said model represents the absence or presence of a biological feature in the test organism or the test biological specimen, wherein
the test organism or the test biological specimen is deemed to have the biological feature when the model score is positive; and
the test organism or the test biological specimen is deemed to not have the biological feature when the model score is negative.

124. The computer program product of claim 123 wherein said biological feature is a disease.

125. The computer program product of claim 124 wherein said disease is cancer.

126. The computer program product of claim 124 wherein said disease is breast cancer, lung cancer, prostate cancer, colorectal cancer, ovarian cancer, bladder cancer, gastric cancer, or rectal cancer.

127. The computer program product of claim 123 wherein
the function of a test in the plurality of tests comprises a ratio between a numerator and a denominator, wherein the numerator comprises a characteristic of a predetermined first cellular constituent in the test organism or the test biological specimen and the denominator comprises a characteristic of a predetermined second cellular constituent in the test organism or the test biological specimen;
the first cellular constituent is more abundant in members of said species or biological specimens that have said biological feature than in members of said species or biological specimens that do not have said biological feature; and
the second cellular constituent is less abundant in members of said species or biological specimens that have said biological feature than in members of said species or biological specimens that do not have said biological feature.

128. The computer program product of claim 119 wherein the plurality of tests comprises a first test and a second test and the identities of the one or more cellular constituents whose characteristics in the test organism or test biological specimen are used to determine the value of the first test are different than the identities of the one or more cellular constituents whose characteristics in the test organism or test biological specimen are used to determine the value of the second test.

129. The computer program product of claim 119 wherein the plurality of tests comprises a first test and a second test and an identity of a cellular constituent in the one or more cellular constituents whose characteristics are used to determine the value of the first test is the same as the identity of a cellular constituent in the one or more cellular constituents whose characteristics are used to determine the value of the second test.

130. The computer program product of claim 129, wherein said first test comprises a ratio between an abundance of a first cellular constituent and an abundance of a second cellular constituent.

131. The computer program product of claim 120 wherein a test in the plurality of tests
contributes a single positive unit to the model score when the test value for the test exceeds the positive threshold assigned to the test;
contributes zero units to the model score when the test value for the test is less than the positive threshold assigned to the test and greater than the negative threshold assigned to the test; and
contributes a single negative unit to the model score when the test value for the test is less than the negative threshold assigned to the test.

132. The computer program product of claim 120 wherein a test in the plurality of tests
contributes a weighted positive unit to the model score when the test value for the test exceeds the positive threshold assigned to the test;
contributes zero units to the model score when the test value for the test is less than the positive threshold assigned to the test and greater than the negative threshold assigned to the test; and
contributes a weighted negative unit to the model score when the test value for the test is less than the negative threshold assigned to the test.

133. The computer program product of claim 132 wherein the magnitude of the weighted positive unit is determined by an amount the test value exceeds the positive threshold assigned to the test.

134. The computer program product of claim 132 wherein the magnitude of the weighted positive unit and the weighted negative unit is determined by a degree of confidence in the test.

135. The computer program product of claim 132 wherein the magnitude of the weighted positive unit and the weighted negative unit is determined by an area under a receiver operating characteristic (ROC) curve used to assign the positive threshold and the negative threshold to the test.

136. The computer program product of claim 132 wherein the magnitude of the weighted negative unit is determined by an amount the test value is less than the negative threshold assigned to the test.

137. The computer program product of claim 119 wherein the species is human.

138. The computer program product of claim 119 wherein the test biological specimen is a biopsy or other form of sample from a tumor, blood, bone, a breast, a lung, a prostate, a colorectum, an ovary, a bladder, a stomach, or a rectum.

139. The computer program product of claim 119, the computer program product further comprising
a cellular constituent data set; and
instructions for using the cellular constituent data set to assign a positive threshold and a negative threshold to a test in said plurality of tests.

140. The computer program product of claim 139 wherein the cellular constituent data set comprises:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism.

141. The computer program product of claim 140 wherein the plurality of cellular constituent characteristic measurements comprises between 5 and 1000 cellular constituent characteristic measurements.

142. The computer program product of claim 140 wherein the plurality of cellular constituent characteristic measurements comprises more than 50 cellular constituent characteristic measurements.

143. The computer program product of claim 140 wherein the plurality of cellular constituent characteristic measurements comprises more than 1000 cellular constituent characteristic measurements.

144. The computer program product of claim 140 wherein said instructions for using the cellular constituent data set to assign a positive threshold and a negative threshold to a test in said plurality of tests comprises selecting:
a first subset of said plurality of cellular constituents, wherein each cellular constituent in said first subset of cellular constituents is up-regulated in organisms in which said biological feature is present; and
a second subset of said plurality of cellular constituents, wherein each cellular constituent in said second subset of cellular constituents is down-regulated in organisms in which said biological feature is present.

145. The computer program product of claim 144, wherein said instructions for using the cellular constituent data set to assign a positive threshold and a negative threshold to a test in said plurality of tests comprises:
constructing a test in said plurality of tests, wherein the function of the test is a ratio between (i) a characteristic of a cellular constituent in said first subset and (ii) a characteristic of a cellular constituent in said second subset.

146. The computer program product of claim 119 wherein a cellular constituent in said plurality of cellular constituents is mRNA, cRNA or cDNA.

147. The computer program product of claim 119 wherein a cellular constituent in said one or more cellular constituents is a nucleic acid or a ribonucleic acid and the characteristic of said cellular constituent is obtained by measuring a transcriptional state of all or a portion of said cellular constituent in said test organism or said test biological specimen.

148. The computer program product of claim 119 wherein a cellular constituent in said one or more cellular constituents is a protein and the characteristic of said cellular constituent is obtained by measuring a translational state of said cellular constituent in said test organism or said test biological specimen.

149. The computer program product of claim 119 wherein the characteristic of a cellular constituent in said one or more cellular constituents is determined using isotope-coded affinity tagging followed by tandem mass spectrometry analysis of the cellular constituent using a sample obtained from the test organism or the test biological specimen.

150. The computer program product of claim 119 wherein the characteristic of a cellular constituent in said one or more cellular constituents is determined by measuring an activity or a post-translational modification of the cellular constituent in a sample obtained from the test organism or in the test biological specimen.

151. The computer program product of claim 119 wherein the plurality of tests consists of two or more tests.

152. The computer program product of claim 119 wherein the plurality of tests consists of between three and ten tests.

153. The computer program product of claim 119, the computer program product further comprising:
instructions for accessing a cellular constituent data set, the cellular constituent data set comprising:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism; and wherein
said instructions for identifying one or more candidate thresholds for each respective test in said plurality of tests comprises:
(i) instructions for computing the function of a respective test in said plurality of tests using the characteristics of the one or more cellular constituents that determine the test value of the respective test, wherein the characteristics of the one or more cellular constituents are from an organism in said plurality of organisms or a biological specimen in said plurality of biological specimens in the cellular constituent data set;
(ii) instructions for repeating said instructions for computing (i) using the characteristics of the one or more cellular constituents that determine the test value from a different organism in said plurality of organisms or said biological specimen in said plurality of biological specimens in the cellular constituent data set;
(iii) instructions for generating a receiver operating characteristic (ROC) curve for said test using the values of the function computed by said instructions for computing (i) and the indication for each organism whose cellular constituent characteristics were used in an instance of said instructions for computing (i);
(iv) instructions for identifying one or more candidate thresholds for the test in the ROC curve; and
(v) instructions for repeating said instructions (i) through (iv) for a different test in said plurality of tests.

154. The computer program product of claim 153 wherein said instruction for repeating (ii) are executed more than ten times.

155. The computer program product of claim 153 wherein said instruction for repeating (ii) are executed more than one hundred times.

156. The computer program product of claim 153 wherein said instruction for repeating (ii) are executed more than one thousand times.

157. The computer program product of claim 153 wherein said instruction for repeating (ii) are executed between ten and twenty thousand times.

158. The computer program product of claim 153 wherein said one or more candidate thresholds for the test in the ROC curve are members of a convex set.

159. The computer program product of claim 154 wherein said convex set is the convex hull of the ROC curve.

160. The computer program product of claim 154 wherein there are between three and ten candidate thresholds in the convex set.

161. The computer program product of claim 119, the computer program product further comprising:
instructions for accessing a cellular constituent data set, wherein said cellular constituent data set comprises:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism; and wherein
said instructions for scoring each candidate threshold combination comprises:
(i) computing a model score for an organism in said plurality of organisms or for a respective organism corresponding to a biological specimen in said plurality of biological specimens using a candidate threshold combination in said plurality of candidate threshold combinations, wherein said computing comprises summing a contribution of each respective test in said model using, for each respective test, the one or more candidate thresholds for the respective test that are specified by the threshold combination;
(ii) repeating said computing for a different organism in said plurality of organisms or for a different respective organism corresponding to a biological specimen in said plurality of biological specimens a number of times; and
(iii) computing a receiver operating characteristic curve based upon the model scores computed in instances of said computing (i) versus the indication whether, for each respective organism in said plurality of organisms or for each respective organism corresponding to a biological specimen in said plurality of biological specimens, said biological feature is present or absent in the respective organism as specified in said cellular constituent data set; and
(iv) assessing a goal function that is determined by said receiver operating characteristic curve.

162. The computer program product of claim 161 wherein said candidate threshold combination specifies a positive threshold and a negative threshold for each test in said plurality of tests.

163. The computer program product of claim 161 wherein said goal function is 7*specificity+sensitivity at a point on the receiver operating characteristic curve that separates model scores that are greater than one from model scores that are less than one wherein
sensitivity=TP/(TP+FN);
specificity=TN/(TN+FP),
wherein
TP=the number of organisms considered by instances of said computing (i) that have said biological feature;
FN=the number of organisms considered by instances of said computing (i) that are falsely identified by said model as having said biological feature at said point on the receiver operating characteristic curve;
TN=the number of organisms considered by instances of said computing (i) that do not have said biological feature; and
FP=the number of organisms considered by instances of said computing (i) that are falsely identified by said model as not having said biological feature at said point on the receiver operating characteristic curve.

164. A computer comprising:
a central processing unit;
a memory, coupled to the central processing unit, the memory storing:
a model characterized by a model score, the model comprising a plurality of tests, wherein each respective test in said plurality of tests is characterized by a test value that is determined by a function of the characteristic of one or more cellular constituents in a plurality of cellular constituents in a test organism of a species or a test biological specimen from an organism of said species;
instructions for identifying one or more candidate thresholds for each respective test in said plurality of tests; and
instructions for scoring each candidate threshold combination in a plurality of candidate threshold combinations, wherein each candidate threshold combination in said plurality of candidate threshold combinations comprises one or more candidate thresholds for each test in said plurality of tests that was identified by said instructions for identifying.

165. The computer of claim 164, the memory further comprising:
instructions for accessing a cellular constituent data set, the cellular constituent data set comprising:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism; and wherein
said instructions for identifying one or more candidate thresholds for each respective test in said plurality of tests comprises:
(i) instructions for computing the function of a respective test in said plurality of tests using the characteristics of the one or more cellular constituents that determine the test value of the respective test, wherein the characteristics of the one or more cellular constituents are from an organism in said plurality of organisms or a biological specimen in said plurality of biological specimens in the cellular constituent data set;
(ii) instructions for repeating said instructions for computing (i) using the characteristics of the one or more cellular constituents that determine the test value from a different organism in said plurality of organisms or said biological specimen in said plurality of biological specimens in the cellular constituent data set;
(iii) instructions for generating a receiver operating characteristic (ROC) curve for said test using the values of the function computed by said instructions for computing (i) and the indication for each organism whose cellular constituent characteristics were used in an instance of said instructions for computing (i);
(iv) instructions for identifying one or more candidate thresholds for the test in the ROC curve; and
(v) instructions for repeating said instructions (i) through (iv) for a different test in said plurality of tests.

166. The computer of claim 164, the memory further comprising:
instructions for accessing a cellular constituent data set, wherein said cellular constituent data set comprises:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism; and wherein
said instructions for scoring each candidate threshold combination comprises:
(i) computing a model score for an organism in said plurality of organisms or for a respective organism corresponding to a biological specimen in said plurality of biological specimens using a candidate threshold combination in said plurality of candidate threshold combinations, wherein said computing comprises summing a contribution of each respective test in said model using, for each respective test, the one or more candidate thresholds for the respective test that are specified by the threshold combination;
(ii) repeating said computing for a different organism in said plurality of organisms or for a different respective organism corresponding to a biological specimen in said plurality of biological specimens a number of times; and
(iii) computing a receiver operating characteristic curve based upon the model scores computed in instances of said computing (i) versus the indication whether, for each respective organism in said plurality of organisms or for each respective organism corresponding to a biological specimen in said plurality of biological specimens, said biological feature is present or absent in the respective organism as specified in said cellular constituent data set; and
(iv) assessing a goal function that is determined by said receiver operating characteristic curve.

167. The computer of claim 166 wherein said goal function is 7*specificity+sensitivity at a point on the receiver operating characteristic curve that separates model scores that are greater than one from model scores that are less than one wherein
sensitivity=TP/(TP+FN);
specificity=TN/(TN+FP),
wherein
TP=the number of organisms considered by instances of said computing (i) that have said biological feature;
FN=the number of organisms considered by instances of said computing (i) that are falsely identified by said model as having said biological feature at said point on the receiver operating characteristic curve;
TN=the number of organisms considered by instances of said computing (i) that do not have said biological feature; and
FP=the number of organisms considered by instances of said computing (i) that are falsely identified by said model as not having said biological feature at said point on the receiver operating characteristic curve.

168. A method comprising:
accessing a model characterized by a model score, the model comprising a plurality of tests, wherein each respective test in said plurality of tests is characterized by a test value that is determined by a function of the characteristic of one or more cellular constituents in a plurality of cellular constituents in a test organism of a species or a test biological specimen from an organism of said species;
identifying one or more candidate thresholds for each respective test in said plurality of tests; and
scoring each candidate threshold combination in a plurality of candidate threshold combinations, wherein each candidate threshold combination in said plurality of candidate threshold combinations comprises one or more candidate thresholds for each test in said plurality of tests that was identified by said instructions for identifying.

169. The method of claim 168, the method further comprising:
accessing a cellular constituent data set, the cellular constituent data set comprising:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism;
and wherein the identifying one or more candidate thresholds for each respective test in said plurality of tests comprises:
(i) computing the function of a respective test in said plurality of tests using the characteristics of the one or more cellular constituents that determine the test value of the respective test, wherein the characteristics of the one or more cellular constituents are from an organism in said plurality of organisms or a biological specimen in said plurality of biological specimens in the cellular constituent data set;
(ii) repeating said computing (i) using the characteristics of the one or more cellular constituents that determine the test value from a different organism in said plurality of organisms or said biological specimen in said plurality of biological specimens in the cellular constituent data set;
(iii) generating a receiver operating characteristic (ROC) curve for said test using the values of the function computed by said instructions for computing (i) and the indication for each organism whose cellular constituent characteristics were used in an instance of said instructions for computing (i);
(iv) identifying one or more candidate thresholds for the test in the ROC curve; and
(v) repeating said computing (i), repeating (ii), generating (iii) and identifying (iv) for a different test in said plurality of tests.

170. The method of claim 168, the method further comprising:
accessing a cellular constituent data set, wherein said cellular constituent data set comprises:
a plurality of cellular constituent characteristic measurements from (i) each organism in a plurality of organisms of said species, or (ii) each biological specimen in a plurality of biological specimens from organisms of said species; and
an indication whether, for each respective organism in said plurality of organisms or for each respective organism corresponding to a biological specimen in said plurality of biological specimens, a biological feature is present or absent in the respective organism;
and wherein said scoring each candidate threshold combination comprises:
(i) computing a model score for an organism in said plurality of organisms or for a respective organism corresponding to a biological specimen in said plurality of biological specimens using a candidate threshold combination in said plurality of candidate threshold combinations, wherein said computing comprises summing a contribution of each respective test in said model using, for each respective test, the one or more candidate thresholds for the respective test that are specified by the threshold combination;
(ii) repeating said computing for a different organism in said plurality of organisms or for a different respective organism corresponding to a biological specimen in said plurality of biological specimens a number of times; and
(iii) computing a receiver operating characteristic curve based upon the model scores computed in instances of said computing (i) versus the indication whether, for each respective organism in said plurality of organisms or for each respective organism corresponding to a biological specimen in said plurality of biological specimens, said biological feature is present or absent in the respective organism as specified in said cellular constituent data set; and
(iv) assessing a goal function that is determined by said receiver operating characteristic curve.

171. The method of claim 170 wherein said goal function is 7*specificity+sensitivity at a point on the receiver operating characteristic curve that separates model scores that are greater than one from model scores that are less than one wherein
sensitivity=TP/(TP+FN);
specificity=TN/(TN+FP)
wherein
TP=the number of organisms considered by instances of said computing (i) that have said biological feature;
FN=the number of organisms considered by instances of said computing (i) that are falsely identified by said model as having said biological feature at said point on the receiver operating characteristic curve;
TN=the number of organisms considered by instances of said computing (i) that do not have said biological feature; and
FP=the number of organisms considered by instances of said computing (i) that are falsely identified by said model as not having said biological feature at said point on the receiver operating characteristic curve.

172. The computer program product of claim 1 wherein the characteristic of a cellular constituent in said one or more cellular constituents is an abundance of said cellular constituent in said test organism of said species or said test biological specimen from said organism of said species.

173. The computer of claim 34 wherein the characteristic of a cellular constituent in said one or more cellular constituents is an abundance of said cellular constituent in said test organism of said species or said test biological specimen from said organism of said species.

174. The computer program product of claim 35 wherein the characteristic of said cellular constituent measured in said biological specimen from a member of all or said portion of said population is an abundance of said cellular constituent.

175. The first computer of claim 75 wherein the characteristic of said cellular constituent measured in said biological specimen from a member of all or said portion of said population is an abundance of said cellular constituent.

176. The method of claim 79 wherein the characteristic of said cellular constituent measured in said biological specimen from a member of all or said portion of said population is an abundance of said cellular constituent.

177. A method comprising:
determining whether a test organism of a species or a test biological specimen from an organism of said species has a biological feature, wherein
the model is characterized by a model score, the model comprising a plurality of tests, wherein
each respective test in said plurality of tests is characterized by a test value that is determined by a function of the characteristics of one or more cellular constituents in a plurality of cellular constituents in said test organism or said test biological specimen from said organism of said species; and
each respective test in the plurality of tests is independently assigned a positive threshold and a negative threshold wherein
the respective test positively contributes to the model score when the test value for the respective test exceeds the positive threshold;
the respective test does not contribute to the model score when the test value for the respective test is less than the positive threshold and greater than the negative threshold; and
the respective test negatively contributes to the model score when the test value for the respective test is less than the negative threshold, wherein
when said model score has a first outcome, said test organism or said test biological specimen has said feature and
when said model score has a second outcome, said test organism or said test biological specimen does not have said feature.

178. The method of claim 177 wherein said first outcome is a positive model score and said second outcome is a negative model score.

179. The method of claim 177 wherein said first outcome is a negative model score and said second outcome is a positive model score.
